SEQUENCE LISTING (2 sequences)

SEQ ID NO: 1 - protein, 465 aa, Homo sapiens
  SIGNAL 1..24
  PROPEP 430..465: note = Removed in mature form
MFLATLYFALPLLDLLLSAEVSGGDRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISDVFQQVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 2 - protein, 1114 aa, Homo sapiens
  SIGNAL 1..28
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

(19) United States
(12) Patent Application Publication
(10) Pub. No.: US 2026/0356810 A1
(43) Pub. Date: Nov. 12, 2026

(54) NOVEL PIPERAZINE-BASED SULFONAMIDES AND THEIR USE AS NEUROPROTECTIVE AND/OR NEURORESTORATIVE AGENTS

(71) Applicant: GENECODE, Tallinn (EE)

(72) Inventors: Delphine CHARVIN, Gex (FR); Serge MIGNANI, Chatenay-Malabry (FR); Frédéric MIEGE, Lyon (FR); Vincent RODESCHINI, Mions (FR); Yves RIBEILL, Durham, NC (US)

(73) Assignee: GENECODE, Tallinn (EE)

(21) Appl. No.: 19/115,920

(22) PCT Filed: Oct. 13, 2023
(86) PCT No.: PCT/EP2023/078567

§ 371 Date: Mar. 27, 2025

(30) Foreign Application Priority Data
Oct. 14, 2022 (EP) 22201683.4

(51) Int. Cl. C07D 295/192; C07D 207/48; C07D 211/96; C07D 307/79

(52) U.S. Cl. CPC C07D 295/192; C07D 207/48; C07D 211/96; C07D 307/79

(57) ABSTRACT

A compound of general formula (I)

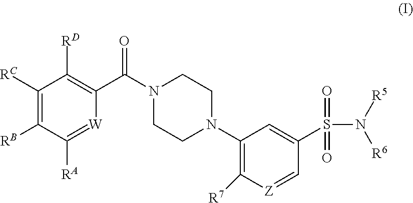

or a pharmaceutically acceptable salt and/or solvate thereof, and the use of the compound of general formula (I) as a neuroprotective and/or a neurorestorative agent, in particular for use in the treatment of a neurological disorder. Also, the process for manufacturing the compound of general formula (I).

### FIELD OF INVENTION

The present invention relates to novel polycyclic sulfonamides comprising at least one unsubstituted and unbridged piperazine. The compounds of the invention are useful as neuroprotective and/or neurorestorative agents, in particular for use in the treatment of neurological disorders.

### BACKGROUND OF INVENTION

Neurological disorders (NDs) are heterogeneous diseases affecting the autonomic, peripheral and central nervous system of the body. Amongst the Central Nervous System (CNS) diseases, Alzheimer's disease (AD), Parkinson's disease (PD), Huntington's disease (HD), Amyotrophic Lateral Sclerosis (ALS), dementia, stroke, head trauma, brain tumor, pain and epilepsy are always the most challenging diseases to be addressed. Compounds actives for treating CNS might also be relevant to other diseases, for example diseases of the peripheral nervous system, eyes, spinal cord and enteric system.

The incidence of NDs is expected to increase dramatically in the 21^{st} century, in particular due to increased life expectancy and demographic changes. Some of these diseases are characterized by age-related gradual decline in neurological functions. In medicine, neurological diseases are the world's important and common cause of disability-adjusted life years, or years of healthy life lost due to death or disability. CNS diseases represent the largest and fastest growing therapeutic domain of unmet medical need, and it is being recognized as a global public health challenge and become a major global health priority. Adequate neurological diagnosis represents an immense challenge, and patients are more concerned about the development of new effective treatments to treat pathophysiology or symptoms. Neurological disorders affect millions of people worldwide and cause permanent damage. They are progressive diseases with symptoms that can degenerate overtime. Although there is generally no definitive cure, supporting treatments exist. The goal of these treatments is mainly to reduce symptoms and preserve the quality of life of the patient as long as possible.

Neurons are postmitotic cells that must live for a lifetime. While young neurons have proper functioning of self-healing protective mechanisms, aging or external or internal insults disturb them, eventually leading to neurodegeneration. These external/internal hazards are traumatic injuries or excitotoxic compounds, reactive oxygen species (ROS), protein aggregates, and other toxic molecules. Fortunately, cells have an intrinsic machinery that blocks death by activating resilience mechanisms or promoting regeneration pathways. Dysfunctionality or insufficiency of these self-healing mechanisms has also been described in neurodegenerative diseases.

Among natural self-healing agents, the glial cell line-derived neurotrophic factor (GDNF) acts as a potent neurotrophic factor, promoting survival in different neuronal populations such as spinal motor neurons, retinal cells, central noradrenergic neurons, or sympathetic neurons, among others. Likewise, GDNF (and other proteins of the GDNF family of neurotrophic factors such as neurturin, artemin and persephin) acts as a powerful trophic factor favoring, not only the survival and plasticity, but also the proliferation, differentiation, and protection of dopaminergic neurons, as well as the synthesis of dopamine and dopaminergic transmission in the developing and adult brain. GDNF can promote neuroprotection through MAP kinase/ERK, Src kinase and PI3 kinase/AKT pathways by inducing several neuroprotective signaling cascade, including the activation of the transcription factor Elk1 through the activation of the GFRα1-RET receptor complex.

The field of applications for these proteins is vast. Pre-clinical and clinical trials have been carried out to evaluate the effect of neurotrophic factors of the GDNF family for the prevention, treatment or management of Parkinson's disease, chronic pain, Alzheimer's disease, amyotrophic lateral sclerosis, neuropathy, depression, stroke, and these proteins were even suggested as male contraceptives. However, the clinical application of GDNF is hampered by its poor pharmacokinetic properties, the fact that it does not cross the blood-brain barrier and thus the necessity for intracranial delivery via stereotaxic surgery, varying biological activity, and high price.

Blood-brain barrier penetrating small-molecule compounds that target the GDNF receptor complex and mimic GDNF biological effects in neurons may be an avenue to overcome these issues and translate to greater efficacy in the clinic. Superior tissue penetration of such compounds could promote survival in all affected neuronal pathways.

WO 2011/070177 A2 (BALTIC TECHNOLOGY DEV LTD) discloses polycyclic compounds for treating neurological disorders. Although these compounds represented a significant improvement at this time, they still have limitations in terms of activity on GFRα1-RET target, solubility, membrane permeability (PAMPA and CaCO₂), intrinsic clearance microsomes and hepatocytes, plasma protein binding, and pharmacokinetic profile. In particular, these compounds are limited in terms of activity on GFRα1-RET target (as evidenced, for example, in luciferase assays).

IVANOVA, L. et al. (ACS OMEGA, Vol. 3, No. 3, 19 Sep. 2018, pp. 11407-11414, ISSN: 2470-1343, DOI: 10.1021/acsomega.8b01524) discloses two compounds, also disclosed in WO 2011/070177 A2, and simulates the dynamic molecular modelling of the interactions between Gglial cell line-derived neurotrophic factor family receptor GFRα1 and these two compounds.

WO 2014/041179 A1 (CHEMEDEST LTD) also disclose the same polycyclic compounds than WO 2011/070177 A2 for treating and/or preventing peripheral neuropathy (peripheric disease), therefore with the same limitations.

It was surprisingly found out by the Applicants that novel polycyclic sulfonamides of formula (I) showed potent GFRα1-RET activity in a luciferase assay, thereby opening the way to overcoming the limitations of available therapeutic solutions.

### SUMMARY

An object of the present invention is a compound of formula (I)

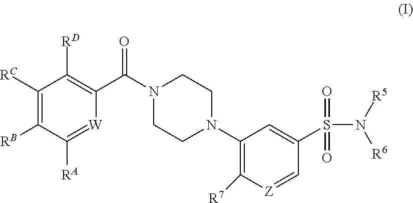

or a pharmaceutically acceptable salt and/or solvate thereof;
   wherein W, R^{A}—R^{D}, Z and R⁵-R⁷ are as defined hereinafter and/or as defined in the claims.

According to one preferred embodiment, the compound of formula (I) is a compound of formula (I-i) or formula (I-ii) as defined hereinafter and/or as defined in the claims.

According to one embodiment, the compound is selected from the compounds of Table 1 herein, and pharmaceutically acceptable salts and/or solvates thereof, provided that the compound is not N,N-diethyl-5-[4-[4-fluoro-2-(trifluoromethyl)benzoyl]piperazin-1-yl]-6-methoxy-pyridine-3-sulfonamide or a pharmaceutically acceptable salt and/or solvate thereof.

Another object of the present invention is a pharmaceutical composition comprising a compound according to the invention and at least one pharmaceutically acceptable carrier.

Another object of the present invention is a compound according to the invention or a pharmaceutical composition according to the invention for use as a medicament. According to one embodiment, the compound or the pharmaceutical composition is for use in the treatment of a neurological disorder.

Another object of the present invention is a process for manufacturing a compound according to the invention.

### DEFINITIONS

In the present invention, the following terms have the following meanings:

### CHEMICAL DEFINITIONS

Where chemical substituents are combinations of chemical groups, the point of attachment of the substituent to the molecule is by the last chemical group recited on the right of the name of the substituent. For example, an arylalkyl substituent is linked to the rest of the molecule through the alkyl moiety and it may by represented as follows: “aryl-alkyl-”.

Unless otherwise indicated, the compounds were named using BIOVIA Draw 2021 (Dassault, France).

The definitions herein referring to the optional or mandatory substitution of a specified group apply both to the substituted group considered as such or to the same group comprised in another chemical moiety, which can both be substituted as set forth herein. For example, “R^{x} represents hydrogen, (C₁-C₈) alkyl, (C₁-C₈) alkyl-O— or cycloalkyl-(C₁-C₈) alkyl-NH—; wherein the alkyl is optionally substituted by at least one F” means that any alkyl group present in the structure of R^{x} may optionally substituted by at least one F, including said (C₁-C₈) alkyl as such (e.g., CF₃), the alkyl comprised in said (C₁-C₈) alkyl-O— (e.g., OCF₃) and the alkyl comprised in said cycloalkyl-(C₁-C₈) alkyl-NH— (e.g., cyclopropyl-CH₂—CHF—CH₂—NH—).

“Alkoxy” refers to an alkyl-O— group.

“Alkyl” refers to a saturated linear or branched hydrocarbon chain, typically comprising from 1 to 16 carbon atoms, preferably from 1 to 12 carbon atoms, more preferably from 1 to 8 carbon atoms, furthermore preferably from 1 to 6 carbon atoms. Alkyl groups may be monovalent or polyvalent (i.e., “alkylene” groups, which are divalent alkyl groups, are encompassed in “alkyl” definition). Non-limiting examples of alkyl groups include methyl, ethyl, n-propyl, i-propyl, n-butyl, i-butyl, s-butyl and t-butyl, pentyl and its isomers (e.g., n-pentyl, iso-pentyl), and hexyl and its isomers (e.g., n-hexyl, iso-hexyl). Particular examples of alkyl groups include methyl, ethyl, n-propyl, i-propyl, n-butyl, s-butyl and t-butyl (including methylene, ethylene, n-propylene, n-butylene and n-butylene).

“Amine” refers to derivatives of ammonia (NH₃), wherein one or more hydrogen atoms have been replaced by a substituent such as, for example, alkyl or aryl. “Amino” refers to the —NH₂ group.

“Aryl” refers to a cyclic, polyunsaturated, aromatic hydrocarbyl group comprising at least one aromatic ring and comprising from 5 to 12 carbon atoms, preferably from 6 to 10 carbon atoms. Aryl groups may be monovalent or polyvalent (e.g., divalent). Aryl groups may have a single ring (e.g., phenyl) or multiple aromatic rings fused together (e.g., naphthyl) or linked covalently. The aromatic ring may optionally include one to two additional rings (either cycloalkyl, heterocycloalkyl or heteroaryl) fused thereto. This definition of “aryl” encompasses the partially hydrogenated derivatives of the carbocyclic systems enumerated herein, as long as at least one ring is aromatic. Aryls may optionally be substituted by at least one group such as, for example, halogen (e.g., F or Cl), (C₁-C₈) alkyl (e.g., methyl) or nitrile (CN). Non-limiting examples of aryl groups include phenyl, biphenyl, biphenylenyl, 5- or 6-tetralinyl, naphthalen-1- or -2-yl, 4-, 5-, 6 or 7-indenyl, 1-2-, 3-, 4- or 5-acenaphthylenyl, 3-, 4- or 5-acenaphthenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, and 1-, 2-, 3—, 4- or 5-pyrenyl. A particular example of aryl group is phenyl.

“Benzylidene” refers to a phenyl group bond to a moiety through an exo carbon-carbon double bound, i.e., ═CH-Ph bond to a carbon atom. The moiety is typically cyclic such as, for example, an heterocycloalkyl.

“Cycloalkyl” refers to a cyclic alkyl group, typically comprising from 3 to 15 carbon atoms, preferably from 3 to 12 carbon atoms, more preferably from 3 to 8 carbon atoms, further more preferably from 3 to 6 carbon atoms. Cycloalkyl groups may be monovalent or polyvalent (e.g., divalent). This definition of “cycloalkyl” encompasses polycyclic cycloalkyls (e.g., bicycles) and bridged cycloalkyl structures, including cycles bound together through one atom (“spiro”) or through two atoms. This definition of “cycloalkyl” encompasses cycloalkyls including a cyclic alkyl group substituted by at least one non-cyclic alkyl, such as, for example, a (C₁-C₈) alkyl (preferably, (C₁-C₄) alkyl, e.g., methyl). Non-limiting examples of cycloalkyl groups include cyclopropyl, cyclobutyl, cyclopentyl, cyclohexyl, cycoheptyl, cyclooctanyl, cyclononanyl, cyclodecanyl, norbornyl, adamantyl, bicyclo[2.2.2]octanyl, bicyclo[4.4.0]decanyl, bicyclo[3.2.1]octanyl, bicyclo[3.3.1]nonanyl, bicyclo[2.1.1]hexane, 2,3-dihydro-1H-indenyl, 1,2,3,4-tetrahydronaphthalenyl, decahydronaphthalenyl, 1,2,3,4-tetrahydronaphthalenyl, and octahydropentalenyl.

“Cₓ-C_{y}” or “(Cₓ-C_{y})” preceding the name of a group means that the group comprises from x to y carbon atoms, in accordance to common terminology in the chemistry field.

“Halogen” refers to a fluorine, chlorine, bromine or iodine atom.

“Heteroalkyl” refers to an alkyl group as defined herein, wherein one or more carbon atoms are replaced by a heteroatom selected from oxygen, nitrogen and sulfur, and wherein the resulting heteroalkyl group comprises at least one carbon atom. In heteroalkyl groups, the heteroatoms are bound along the alkyl chain only to carbon atoms, i.e., each heteroatom is separated from any other heteroatom by at least one carbon atom, typically by at least two carbon atoms. Heteroalkyl groups may be monovalent or polyvalent (e.g., divalent). The nitrogen and sulfur heteroatoms may optionally be oxidized and the nitrogen heteroatoms may optionally be quaternized (e.g., sulfur may be oxidized as SO or SO₂). Heteroalkyl groups may further include one or more ═O and/or ═S groups. In one embodiment, at least two carbon atoms are replaced by a heteroatom. In one embodiment, the heteroalkyl is bound to another group or molecule through a carbon atom, i.e., the binding atom is not selected among the heteroatoms included therein. In one embodiment, the heteroalkyl is bound to another group or molecule through one of the heteroatoms included therein. When substituted by one or more other group(s), an heteroalkyl may be substituted either through a carbon atom or through a heteroatom (e.g., nitrogen), unless otherwise specified. Non-limiting examples of heteroalkyl include alkoxy, ethers and polyethers (e.g., polyethylene glycol), secondary and tertiary amines and polyamines, thioethers and polythioethers, and combinations thereof.

“Heteroaryl” refers to aromatic rings or aromatic ring systems comprising from 5 to 15 carbon atoms, preferably from 4 to 12 carbon atoms, more preferably from 3 to 10 carbon atoms, having one or two rings that are fused together or linked covalently, wherein at least one ring is aromatic, and wherein one or more carbon atoms in one or more of these rings is replaced by oxygen, nitrogen and/or sulfur atoms. Heteroaryl groups may be monovalent or polyvalent (e.g., divalent). The nitrogen and sulfur heteroatoms may optionally be oxidized and the nitrogen heteroatoms may optionally be quaternized (e.g., the heteroatom is substituted by oxo (═O) for sulfur atom or (→O) for nitrogen atom). This definition of “heteroaryl” encompasses the partially hydrogenated derivatives of the carbocyclic systems enumerated herein, as well as ring systems including one or more fused non-aromatic cycloalkyl and/or heterocycloalkyl ring(s), as long as at least one ring is aromatic. In one embodiment, the heteroaryl is bound to another group or molecule through a carbon atom, i.e., the binding atom is not selected among the heteroatoms included therein. In one embodiment, the heteroaryl is bound to another group or molecule through one of the heteroatoms included therein. When substituted by one or more other group(s), an heteroaryl may be substituted either through a carbon atom or through a heteroatom (e.g., nitrogen), unless otherwise specified. Heteroaryls may optionally be substituted by at least one group such as, for example, halogen (e.g., F or Cl), (C₁-C₈) alkyl (preferably, (C₁-C₄) alkyl, e.g., methyl) or nitrile (CN). Non-limiting examples of heteroaryl groups include pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, tetrazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl and quinoxalinyl. Non-limiting examples of heteroaryl groups comprising at least one fused non-aromatic ring include 2,3-dihydrobenzofuranyl, benzo[d][1,3]dioxolyl, indolinyl, 2,3-dihydrobenzo[b][1,4]dioxinyl, 3,4-dihydro-2H-benzo[b][1,4]oxazinyl, 1,2,3,4-tetrahydroquinoxaline, 3,4-dihydro-2H-benzo[b][1,4]thiazine and 2,3-dihydrobenzo[b][1,4]oxathiine.

“Heteroarylidene” refers to a heteroaryl group bond to a moiety through an exo carbon-carbon double bound, i.e., ═CH-heteroaryl bond to a carbon atom. The moiety is typically cyclic such as, for example, an heterocycloalkyl.

“Heterocycloalkyl” refers to a cyclic heteroalkyl group, typically comprising from 2 to 15 carbon atoms, preferably from 2 to 11 carbon atoms, more preferably from 2 to 7 carbon atoms, furthermore preferably from 2 to 6 carbon atoms. Heterocycloalkyl groups may be monovalent or polyvalent (e.g., divalent). Heterocycloalkyl groups are typically 3- to 7-membered, preferably 5- or 6-membered. Heterocycloalkyl are typically monocyclic or bicyclic, preferably monocyclic. This definition encompasses polycyclic heterocycloalkyls (e.g., bicycles) and bridged heterocycloalkyl structures, including cycles bound together through one atom (“spiro”) or through two atoms. The nitrogen and sulfur heteroatoms may optionally be oxidized and the nitrogen heteroatoms may optionally be quaternized (e.g., the heteroatom is substituted by oxo (═O) for sulfur atom or (→O) for nitrogen atom). In one embodiment, the heterocycloalkyl is bound to another group or molecule through a carbon atom, i.e., the binding atom is not selected among the heteroatoms included therein. In one embodiment, the heterocycloalkyl is bound to another group or molecule through one of the heteroatoms included therein. When substituted by one or more other group(s), an heterocycloalkyl may be substituted either through a carbon atom or through a heteroatom (e.g., nitrogen), unless otherwise specified. Heterocycloalkyls may optionally be substituted by at least one group such as, for example, halogen (e.g., F or Cl), (C₁-C₈) alkyl (preferably, (C₁-C₄) alkyl, e.g., methyl), nitrile (CN) or ═O. Non-limiting examples of heterocycloalkyl include aziridine, pyrrolidine, piperidine, piperazine, morpholine, thiomorpholine, azepane, azocane, octahydro-1H-isoindole, decahydroisoquinoline, tetrahydrofuran, tetrahydropyran, tetrahydroisoquinoline (e.g., 1,2,3,4-tetrahydroisoquiline), hexahydropyridazine, hexahydropyrazine, hexahydropyrimidine, decahydroquinoline, octahydropyrrolo[3,4-c]pyrrole, isoindoline, 1,2,3,4-tetrahydroquinoline and oxetane.

“Prodrug” refers to a pharmacologically acceptable derivative of a therapeutic agent (e.g., a compound according to the invention) whose in vivo biotransformation product is the therapeutic agent (active drug). Prodrugs are typically characterized by increased bioavailability and are readily metabolized in vivo into the active compounds. Non-limiting examples of prodrugs include amide prodrugs and carboxylic acid ester prodrugs.

“Solvate” refers to molecular complex comprising a compound along with stoichiometric or sub-stoichiometric amounts of one or more molecules of one or more solvents, typically the solvent is a pharmaceutically acceptable solvent such as, for example, ethanol. The term “hydrate” refers to a solvate when the solvent is water (H₂O).

“Ylidene” refers to CH group involved in an exo carbon-carbon double bound with another moiety. The moiety is typically cyclic such as, for example, an heterocycloalkyl.

### GENERAL DEFINITIONS

“About” is used herein to mean approximately, roughly, around, or in the region of.

The term “about” preceding a figure means plus or less 10% of the value of the figure. When the term “about” is used in conjunction with a numerical range, it modifies that range by extending the boundaries above and below the numerical values set forth by 10%.

“Administration”, or a variant thereof (e.g., “administering”), means providing a therapeutic agent alone or as part of a pharmaceutically acceptable composition, to the patient in whom/which the condition, symptom, or disease is to be treated.

“Comprise” or a variant thereof (e.g., “comprises”, “comprising”) is used herein according to common patent application drafting terminology. Hence, “comprise” preceded by an object and followed by a constituent means that the presence of a constituent in the object is required (typically as a component of a composition), but without excluding the presence of any further constituent(s) in the object. Moreover, any occurrence of “comprise” or a variant thereof herein also encompasses narrower expression “substantially consist of”, further narrower expression “consist of” and any variants thereof (e.g., “consists of”, “consisting of”), and may be replaced thereby, unless otherwise stated.

“GDNF family receptor alpha-1”, “GFRα1”, or “GDNFRα1”, also named “RET ligand 1” or “TGF-beta-related neurotrophic factor receptor 1”, is a protein from the GDNFR family that acts as a receptor for GDNF. It mediates the GDNF-induced autophosphorylation and activation of the RET receptor. In humans, GFRα1 is encoded by the GFRA1 gene. An exemplary amino acid sequence of human GFRα1 is given in SEQ ID NO: 1, in which amino acid residues 1-24 correspond to the signal peptide and amino acid residues 430-465 correspond to the propeptide which is removed in mature form.

SEQ ID NO: 1
MFLATLYFALPLLDLLLSAEVSGGDRLDCVKASDQCLKEQSCSTKYRTLR
QCVAGKETNFSLASGLEAKDECRSAMEALKQKSLYNCRCKRGMKKEKNCL
RIYWSMYQSLQGNDLLEDSPYEPVNSRLSDIFRVVPFISDVFQQVEHIPK
GNNCLDAAKACNLDDICKKYRSAYITPCTTSVSNDVCNRRKCHKALRQFF
DKVPAKHSYGMLFCSCRDIACTERRRQTIVPVCSYEEREKPNCLNLQDSC
KTNYICRSRLADFFTNCQPESRSVSSCLKENYADCLLAYSGLIGTVMTPN
YIDSSSLSVAPWCDCSNSGNDLEECLKFLNFFKDNTCLKNAIQAFGNGSD
VTVWQPAFPVQTTTATTTTALRVKNKPLGPAGSENEIPTHVLPPCANLQA
QKLKSNVSGNTHLCISNGNYEKEGLGASSHITTKSMAAPPSCGLSPLLVL
VVTALSTLLSLTETS

“Human” refers to a male or female human subject at any stage of development, including neonate, infant, juvenile, adolescent and adult.

“Neuroprotective” refers to the protection of a neuronal cell from insults, events, or conditions that would normally result in a loss of neuronal cell's functions, and ultimately, neuronal cell death. Such insults, events, or conditions include, without limitation, neuronal stress, for instance, caused by hypoxia or ischemia; traumatic injuries; and exposure to toxic molecules, for instance, to abnormal misfolded proteins, protein aggregates, excitotoxins, reactive oxygen species, endoplasmic reticulum stressors, mitochondrial stressors, Golgi apparatus antagonists and the like. The term also characterizes the detectable biological activity of a compound in reducing the amount or level of neuronal cell's loss of functions and/or neuronal cell death.

“Neurorestorative” refers to the restoration or rescue of a neuronal cell and in particular of its functions, from the effect of an insult, event, or condition that would normally result in a loss of neuronal cell's functions if not in neuronal cell death.

“Patient” refers to a subject who/which is awaiting the receipt of, or is receiving medical care or was/is/will be the object of a medical procedure, or is monitored for the development of the targeted disease or condition, such as, for example, a neurological disorder.

“Pharmaceutically acceptable” means that the ingredients of a composition are compatible with each other and not deleterious to the subject to which/whom it is administered.

“Pharmaceutically acceptable carrier” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, e.g., FDA Office or EMA. Examples of pharmaceutically acceptable carriers include, but are not limited to, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene— block polymers, polyethylene glycol and wool fat.

“Pharmaceutical composition” refers to a composition comprising at least one therapeutic agent (e.g., a compound according to the present invention) and at least one pharmaceutically acceptable carrier.

“Proto-oncogene tyrosine-protein kinase receptor Ret”, or in short “RET”, also named “cadherin family member 12”, is a receptor tyrosine kinase involved in numerous cellular mechanisms including cell proliferation, neuronal navigation, cell migration, and cell differentiation. RET is activated upon (i) binding of a neurotrophic factor of the GDNF family (e.g., GDNF, neurturin, artemin or persephin) to a receptor of the GDNFR family (e.g., GFRα1, GFRα2, GFRα3 or GFRα4), then (ii) complex formation between RET and the receptor of the GDNFR family, (iii) dimerization and (iv) trans-autophosphorylation. An exemplary amino acid sequence of human RET is given in SEQ ID NO: 2, in which amino acid residues 1-28 correspond to the signal peptide.

SEQ ID NO: 2
MAKATSGAAGLRLLLLLLLPLLGKVALGLYFSRDAYWEKLYVDQAAGTPL
LYVHALRDAPEEVPSFRLGQHLYGTYRTRLHENNWICIQEDTGLLYLNRS
LDHSSWEKLSVRNRGFPLLTVYLKVFLSPTSLREGECQWPGCARVYFSFF
NTSFPACSSLKPRELCFPETRPSFRIRENRPPGTFHQFRLLPVQFLCPNI
SVAYRLLEGEGLPFRCAPDSLEVSTRWALDREQREKYELVAVCTVHAGAR
EEVVMVPFPVTVYDEDDSAPTFPAGVDTASAVVEFKRKEDTVVATLRVFD
ADVVPASGELVRRYTSTLLPGDTWAQQTFRVEHWPNETSVQANGSFVRAT
VHDYRLVLNRNLSISENRTMQLAVLVNDSDFQGPGAGVLLLHFNVSVLPV
SLHLPSTYSLSVSRRARRFAQIGKVCVENCQAFSGINVQYKLHSSGANCS
TLGVVTSAEDTSGILFVNDTKALRRPKCAELHYMVVATDQQTSRQAQAQL
LVTVEGSYVAEEAGCPLSCAVSKRRLECEECGGLGSPTGRCEWRQGDGKG
ITRNFSTCSPSTKTCPDGHCDVVETQDINICPQDCLRGSIVGGHEPGEPR
GIKAGYGTCNCFPEEEKCFCEPEDIQDPLCDELCRTVIAAAVLFSFIVSV
LLSAFCIHCYHKFAHKPPISSAEMTFRRPAQAFPVSYSSSGARRPSLDSM
ENQVSVDAFKILEDPKWEFPRKNLVLGKTLGEGEFGKVVKATAFHLKGRA
GYTTVAVKMLKENASPSELRDLLSEFNVLKQVNHPHVIKLYGACSQDGPL
LLIVEYAKYGSLRGFLRESRKVGPGYLGSGGSRNSSSLDHPDERALTMGD
LISFAWQISQGMQYLAEMKLVHRDLAARNILVAEGRKMKISDFGLSRDVY
EEDSYVKRSQGRIPVKWMAIESLFDHIYTTQSDVWSFGVLLWEIVTLGGN
PYPGIPPERLFNLLKTGHRMERPDNCSEEMYRLMLQCWKQEPDKRPVFAD
ISKDLEKMMVKRRDYLDLAASTPSDSLIYDDGLSEEETPLVDCNNAPLPR
ALPSTWIENKLYGMSDPNWPGESPVPLTRADGTNTGFPRYPNDSVYANWM
LSPSAAKLMDTFDS

“Selected from” is used herein according to common patent application drafting terminology, to introduce a list of elements among which one or more item(s) is (are) selected. Any occurrence of “selected from” in the specification may be replaced by “selected from the group comprising or consisting of” and reciprocally without changing the meaning thereof.

“Subject” refers to an animal, typically a warm-blooded animal, preferably a mammal, more preferably a primate, furthermore preferably a human. In one embodiment, the subject is a “patient” as defined herein. In one embodiment, the subject is affected, preferably is diagnosed, with a disease. In one embodiment, the subject is at risk of developing a disease. Examples of risks factor include, but are not limited to, genetic predisposition, or familial history of the disease.

“Therapeutic agent”, “active pharmaceutical ingredient” and “active ingredient” refer to a compound for therapeutic use and relating to health. Especially, a therapeutic agent (e.g., a compound according to the present invention) may be indicated for treating a disease (e.g., a neurological disorder). An active ingredient may also be indicated for improving the therapeutic activity of another therapeutic agent.

“Therapeutically effective amount” (in short “effective amount”) refers to the amount of a therapeutic agent (e.g., a compound according to the present invention) that is sufficient to achieve the desired therapeutic, prophylactic or preventative effect in the patient to which/whom it is administered, without causing significant negative or adverse side effects to said patient. A therapeutically effective amount may be administered prior to the onset of the disease for a prophylactic or preventive action. Alternatively, or additionally, the therapeutically effective amount may be administered after initiation of the disease for a therapeutic action.

“Treating”, “treatment” or “alleviation” refers to both therapeutic treatment and prophylactic or preventative measures, wherein the object is to prevent or slow down (lessen) the targeted pathologic condition or disorder (herein a “disease”) (e.g., a neurological disorder). Those in need of treatment include those already with the disease as well as those prone to have the disease or those in whom the condition or disease is to be prevented. A patient is successfully “treated” for a disease if, after receiving a therapeutic amount of a therapeutic agent (e.g., a compound according to the present invention), the patient shows observable and/or measurable reduction in or absence of one or more of the following: reduction in the number of pathogens; reduction in the percent of total cells that are pathogenic; and/or relief to some extent, one or more of the symptoms associated with the specific disease; reduced morbidity and mortality, and improvement in quality of life issues. The above parameters for assessing successful treatment and improvement in the disease are readily measurable by routine procedures familiar to a physician.

### DETAILED DESCRIPTION

### Compound

An object of the present invention is a compound of formula (I)

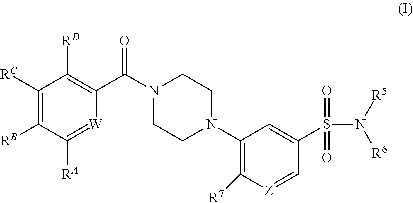

or a pharmaceutically acceptable salt and/or solvate thereof;
wherein
W represents CH or N;
R^{A}, R^{B}, R^{C} and R^{D} each independently represents hydrogen, F, Cl, CH₃, CF₃, CHF₂ or CH₂F;
R⁷ represents hydrogen, OH, halogen, (C₁-C₈) alkyl, cycloalkyl, (C₁-C₈) alkyl-O—, cycloalkyl-O—, cycloalkyl-(C₁-C₈) alkyl-O—, heterocycloalkyl-O—, heterocycloalkyl-(C₁-C₈) alkyl-O—, aryl-(C₁-C₈) alkyl-O—, heteroaryl-(C₁-C₈) alkyl-O—, R¹¹O—(C₁-C₈) alkyl-O—, R¹¹R¹²N—(C₁-C₈) alkyl-O—, (R¹¹O)(R¹²)N—(C₁-C₈) alkyl-O—, R¹¹R¹²N—(C₁-C₈) alkyl-, R¹¹O—(C₁-C₈) alkyl-, NR¹¹R¹², CN, CO₂H, CO₂R¹¹, CONH₂, CON(R¹¹)H, heterocycloalkyl, or heteroaryl; wherein R¹¹ and R¹² each independently represents hydrogen or (C₁-C₈) alkyl;
   wherein the alkyl or cycloalkyl in R⁷ is optionally substituted by at least one F, Cl, OH, ═O, (C₁-C₈) alkyl, (C₁-C₈) alkyl-O—, heterocycloalkyl, aryl or heteroaryl;
   wherein the heterocycloalkyl, aryl or heteroaryl (i.e., any heterocycloalkyl, aryl or heteroaryl that is represented by R⁷ or that is part of any substituent thereof) is optionally substituted by at least one F, Cl, (C₁-C₈) alkyl, CF₃, CHF₂, CH₂F, OCF₃, CN, OH, ═O, →O, (C₁-C₈) alkoxy, NR¹³R¹⁴, R¹³R¹⁴N—(C₁-C₈) alkyl-, R³O₂C—(C₁-C₈) alkyl-, CO₂H, R¹³R¹⁴N—C(O)—, R¹³O—NR¹⁴—, or (C₁-C₈) alkyl-CO₂—;
   wherein R¹³ and R¹⁴ each independently represents hydrogen or (C₁-C₈) alkyl;
Z represents C—H, C—R⁸ or N;
   wherein R⁸ represents (C₁-C₄) alkyl, F, Cl, CF₃, CHF₂, CH₂F, OCF₃, CN, OH or (C₁-C₄) alkoxy;
or Z represents C—R⁸ and R⁷ and R⁸ form together with the carbon atoms to which they are bound a cycloalkyl or heterocycloalkyl,
   wherein the cycloalkyl or heterocycloalkyl is optionally substituted by at least one F, OH, ═O, →O, (C₁-C₈) alkyl, CF₃, HO₂C—CH₂—, (C₁-C₄) alkyl-CO₂—CH₂—, R¹⁵R¹⁶N—CH₂—, aryl, or aryl-(C₁-C₈) alkyl-, wherein R¹⁵ and R¹⁶ each independently represents hydrogen or (C₁-C₈) alkyl;
R⁵ represents hydrogen, (C₁-C₈) alkyl, CH₃ substituted by one to three (C₁-C₈) alkyl groups, cycloalkyl, heterocycloalkyl, aryl, or cycloalkyl-(C₁-C₈) alkyl;
   wherein the alkyl or cycloalkyl in R⁵ is optionally substituted by at least one F, Cl, (C₁-C₈) alkyl, CF₃, OCF₃, CN, OH, ═O, (C₁-C₈) alkoxy, NR¹⁷R¹⁸, CO₂H, R¹⁷R¹⁸N—C(O)—, R¹⁷O—NR¹⁸—, heterocycloalkyl, aryl or heteroaryl; wherein R¹⁷ and R¹⁸ each independently represents hydrogen or (C₁-C₈) alkyl;
   wherein the heterocycloalkyl, aryl or heteroaryl (i.e., any heterocycloalkyl, aryl or heteroaryl that is represented by R⁵ or that is part of any substituent thereof) is optionally substituted by at least one F, Cl, (C₁-C₈) alkyl, CF₃, CHF₂, CH₂F, OCF₃, CN, OH, ═O, →O, (C₁-C₈) alkoxy, NR¹⁹R²⁰, CO₂H, R¹⁹R²⁰N—C(O)—, R¹⁹O—NR²⁰—, (C₁-C₈) alkyl-CO₂—, R¹⁹R²⁰N—(C₁-C₈) alkyl-, R¹⁹O₂C—(C₁-C₈) alkyl-, heterocycloalkyl, heteroaryl, aryl, or aryl-(C₁-C₈) alkyl-; wherein R¹⁹ and R²⁰ each independently represents hydrogen or (C₁-C₈) alkyl;
R⁶ represents (C₁-C₈) alkyl, CH₃ substituted by one to three (C₁-C₈) alkyl groups, cycloalkyl, heterocycloalkyl, aryl, or cycloalkyl-(C₁-C₈) alkyl;
   wherein the alkyl or cycloalkyl in R⁶ is optionally substituted by at least one F, Cl, (C₁-C₈) alkyl, CF₃, OCF₃, CN, OH, ═O, (C₁-C₈) alkoxy, NR²¹R²², CO₂H, R²¹R²²N—C(O)—, R²¹O—NR²²—, heterocycloalkyl, aryl or heteroaryl; wherein R²¹ and R²² each independently represents hydrogen or (C₁-C₈) alkyl;
   wherein the heterocycloalkyl, aryl or heteroaryl (i.e., any heterocycloalkyl, aryl or heteroaryl that is represented by R⁶ or that is part of any substituent thereof) is optionally substituted by at least one F, Cl, (C₁-C₈) alkyl, CF₃, CHF₂, CH₂F, OCF₃, CN, OH, ═O, →O, (C₁-C₈) alkoxy, NR²³R²⁴, CO₂H, R²³R²⁴N—C(O)—, R²³O—NR²⁴—, (C₁-C₈) alkyl-CO₂—, R²³R²⁴N—(C₁-C₈) alkyl-, R²³O₂C—(C₁-C₈) alkyl-, heterocycloalkyl, heteroaryl, aryl, or aryl-(C₁-C₈) alkyl-; wherein R²³ and R²⁴ each independently represents hydrogen or (C₁-C₈) alkyl;
or R⁵ and R⁶ form together with the nitrogen atom to which they are bound an heterocycloalkyl,
   wherein the heterocycloalkyl (i.e., the heterocycloalkyl that is formed by R⁵, R⁶ and the nitrogen atom to which they are bound) is optionally substituted by at least one F, Cl, (C₁-C₈) alkyl, CF₃, OCF₃, CN, OH, ═O, →O, (C₁-C₈) alkoxy, NR²⁵R²⁶, CO₂H, (C₁-C₈) alkyl-CO₂—, R²⁵R²⁶N—C(O)—, R²⁵O—NR²⁶—, R²⁵R²⁶N—(C₁-C₈) alkyl-, R²O₂C—(C₁-C₈) alkyl-, cycloalkyl, heterocycloalkyl, aryl, heteroaryl, cycloalkyl-(C₁-C₈) alkyl-, heterocycloalkyl-(C₁-C₈) alkyl-, aryl-(C₁-C₈) alkyl-, heteroaryl-(C₁-C₈) alkyl-, aryl-cycloalkyl-, cycloalkyl-O—, heterocycloalkyl-O—, aryl-O—, heteroaryl-O—, cycloalkyl-(C₁-C₈) alkyl-O—, heterocycloalkyl-(C₁-C₈) alkyl-O—, aryl-(C₁-C₈) alkyl-O—, heteroaryl-(C₁-C₈) alkyl-O—, cycloalkyl-NR²⁵—, heterocycloalkyl-NR²⁵—, aryl-NR²⁵—, heteroaryl-NR²⁵—, cycloalkyl-(C₁-C₈) alkyl-NR²⁵—, heterocycloalkyl-(C₁-C₈) alkyl-NR²⁵—, aryl-(C₁-C₈) alkyl-NR²⁵—, heteroaryl-(C₁-C₈) alkyl-NR²⁵—, benzylidene, heteroarylidene, aryl-(C₁-C₈) alkyl-ylidene- or heteroaryl-(C₁-C₈) alkyl-ylidene-;
   wherein R²⁵ and R²⁶ each independently represents hydrogen or (C₁-C₈) alkyl;
   wherein the heterocycloalkyl, aryl, heteroaryl, benzylidene or heteroarylidene (i.e., any heterocycloalkyl, aryl, heteroaryl, benzylidene or heteroarylidene that is part of any substituent of the heterocycloalkyl formed by R⁵, R⁶ and the nitrogen atom to which they are bound) is optionally substituted at least one F, Cl, (C₁-C₈) alkyl, CF₃, CHF₂, CH₂F, OCF₃, CN, OH, ═O, →O, (C₁-C₈) alkoxy, NR²⁷R²⁸, CO₂H, R²⁷R²⁸N—C(O)—, R²⁷O—NR²⁸—, (C₁-C₈) alkyl-CO₂—, R²⁷R²⁸N—(C₁-C₈) alkyl-, R²⁷O₂C—(C₁-C₈) alkyl-, heterocycloalkyl, heteroaryl, aryl, or aryl-(C₁-C₈) alkyl-; wherein R²⁷ and R²⁸ each independently represents hydrogen or (C₁-C₈) alkyl.

According to one preferred embodiment, when exactly two groups selected from R^{A}, R^{B} and R^{C} represent hydrogen, R^{D} represents CF₃, R⁷ represents OCH₃ and Z represents C—H, then the remaining group among R^{A}, R^{B} and R^{C} does not represent F.

In any one of the following embodiments directed to specific limitations to the structure of the compounds of formula (I), any alkyl, heteroalkyl, cycloalkyl, heterocycloalkyl, aryl, heteroaryl, benzylidene or heteroarylidene may independently be optionally substituted as indicated under formula (I) herein, unless otherwise specified.

According to one preferred embodiment, R^{D} represents hydrogen, F, Cl, CH₃, CHF₂ or CH₂F. In this embodiment, the formula (I) is noted “formula (I-i)”.

According to one preferred embodiment, R⁷ represents hydrogen, OH, halogen, (C₁-C₈) alkyl, cycloalkyl, (C₂-C₈) alkyl-O—, cycloalkyl-O—, cycloalkyl-(C₁-C₈) alkyl-O—, heterocycloalkyl-O—, heterocycloalkyl-(C₁-C₈) alkyl-O—, aryl-(C₁-C₈) alkyl-O—, heteroaryl-(C₁-C₈) alkyl-O—, R¹¹O—(C₁-C₈) alkyl-O—, R¹¹R¹²N—(C₁-C₈) alkyl-O—, (R¹¹O)(R¹²)N—(C₁-C₈) alkyl-O—, R¹¹R¹²N—(C₁-C₈) alkyl-, R¹¹O—(C₁-C₈) alkyl-, NR¹¹R¹², CN, CO₂H, CO₂R¹¹, CONH₂, CON(R¹¹)H, heterocycloalkyl, or heteroaryl; wherein R¹¹ and R¹² each independently represents hydrogen or (C₁-C₈) alkyl; wherein the alkyl, cycloalkyl, heterocycloalkyl, aryl or heteroaryl is optionally substituted as defined under formula (I) hereinabove. In this embodiment, the formula (I) is noted “formula (I-ii)”.

According to one preferred embodiment, the present invention relates to a compound of formula (I) or a pharmaceutically acceptable salt and/or solvate thereof; wherein
W represents CH or N;
R^{A}, R^{B}, R^{C} and R^{D} each independently represents hydrogen, F, Cl, CH₃, CF₃, CHF₂ or CH₂F,
R⁷ represents hydrogen, OH, halogen, (C₁-C₈) alkyl, cycloalkyl, (C₁-C₈) alkyl-O—, cycloalkyl-O—, cycloalkyl-(C₁-C₈) alkyl-O—, heterocycloalkyl-O—, R¹¹O—(C₁-C₈) alkyl-O—, R¹¹R¹²N—(C₁-C₈) alkyl-O— or (R¹¹O)(R¹²)N—(C₁-C₈) alkyl-O—; wherein R¹¹ and R¹² each independently represents hydrogen or (C₁-C₈) alkyl;
   wherein the alkyl or cycloalkyl is optionally substituted by at least one F, Cl, heterocycloalkyl, aryl or heteroaryl;
      wherein the heterocycloalkyl, aryl or heteroaryl is optionally substituted by at least one F, Cl, CF₃, CHF₂, CH₂F, OCF₃, CN, OH, (C₁-C₈) alkoxy, NR¹³R¹⁴, CO₂H, R¹³R¹⁴N—C(O)—, or R¹³O—NR¹⁴—; wherein R¹³ and R¹⁴ each independently represents hydrogen or (C₁-C₈) alkyl;
Z represents C—H or N;
or Z represents C—R⁸ and R⁷ and R⁸ form together with the carbon atoms to which they are bound a cycloalkyl or heterocycloalkyl,
   wherein the cycloalkyl or heterocycloalkyl is optionally substituted by at least one F, OH, CF₃, HO₂C—CH₂—, (C₁-C₄) alkyl-CO₂—CH₂—, or R¹⁵R¹⁶N—CH₂—, wherein R¹⁵ and R¹⁶ each independently represents hydrogen or (C₁-C₈) alkyl;
R⁵ represents hydrogen, (C₁-C₈) alkyl, CH₃ substituted by one to three (C₁-C₈) alkyl groups, cycloalkyl or cycloalkyl-(C₁-C₈) alkyl;
   wherein the alkyl or cycloalkyl is optionally substituted by at least one F, Cl, CF₃, OCF₃, CN, OH, (C₁-C₈) alkoxy, NR¹⁷R¹⁸, CO₂H, R¹⁷R¹⁸N—C(O)—, R¹⁷O—NR¹⁸—, heterocycloalkyl, aryl or heteroaryl; wherein R¹⁷ and R¹⁸ each independently represents hydrogen or (C₁-C₈) alkyl;
      wherein the heterocycloalkyl, aryl or heteroaryl is optionally substituted by at least one F, Cl, CF₃, CHF₂, CH₂F, OCF₃, CN, OH, (C₁-C₈) alkoxy, NR¹⁹R²⁰, CO₂H, R¹⁹R²⁰N—C(O)—, or R¹⁹—NR²⁰—; wherein R¹⁹ and R²⁰ each independently represents hydrogen or (C₁-C₈) alkyl;
R⁶ represents (C₁-C₈) alkyl, CH₃ substituted by one to three (C₁-C₈) alkyl groups, cycloalkyl or cycloalkyl-(C₁-C₈) alkyl;
   wherein the alkyl or cycloalkyl is optionally substituted by at least one F, Cl, CF₃, OCF₃, CN, OH, (C₁-C₈) alkoxy, NR²¹R²², CO₂H, R²¹R²²N—C(O)—, R²¹O—NR²²—, heterocycloalkyl, aryl or heteroaryl; wherein R²¹ and R²² each independently represents hydrogen or (C₁-C₈) alkyl;
      wherein the heterocycloalkyl, aryl or heteroaryl is optionally substituted by at least one F, Cl, CF₃, CHF₂, CH₂F, OCF₃, CN, OH, (C₁-C₈) alkoxy, NR²³R²⁴, CO₂H, R²³R²⁴N—C(O)—, or R²³O—NR²⁴—; wherein R²³ and R²⁴ each independently represents hydrogen or (C₁-C₈) alkyl;
or R⁵ and R⁶ form together with the nitrogen atom to which they are bound an heterocycloalkyl,
   wherein the heterocycloalkyl is optionally substituted by at least one F, Cl, CF₃, OCF₃, CN, OH, (C₁-C₈) alkoxy, NR²⁵R²⁶, CO₂H, R²⁵R²⁶N—C(O)—, R²⁵O—NR²⁶—, heterocycloalkyl, aryl, heteroaryl, heterocycloalkyl-(C₁-C₈) alkyl-, aryl-(C₁-C₈) alkyl-, heteroaryl-(C₁-C₈) alkyl-, cycloalkyl-(C₁-C₈) alkyl-O—, aryl-(C₁-C₈) alkyl-O—, heteroaryl-(C₁-C₈) alkyl-O—, cycloalkyl-(C₁-C₈) alkyl-NR²⁵—, aryl-(C₁-C₈) alkyl-NR²⁵—, heteroaryl-(C₁-C₈) alkyl-NR²⁵—, benzylidene, heteroarylidene, aryl-(C₁-C₈) alkyl-ylidene- or heteroaryl-(C₁-C₈) alkyl-ylidene-;
      wherein R²⁵ and R²⁶ each independently represents hydrogen or (C₁-C₈) alkyl;
      wherein the heterocycloalkyl, aryl, heteroaryl, benzylidene or heteroarylidene is optionally substituted at least one F, Cl, CF₃, CHF₂, CH₂F, OCF₃, CN, OH, (C₁-C₈) alkoxy, NR²⁷R²⁸, CO₂H, R²⁷R²⁸N—C(O)—, or R²⁷O—NR²⁸—; wherein R²⁷ and R²⁸ each independently represents hydrogen or (C₁-C₈) alkyl;
provided that, when exactly two groups selected from R^{A}, R^{B} and R^{C} represent hydrogen, R^{D} represents CF₃, R⁷ represents OCH₃ and Z represents C—H, then the remaining group among R^{A}, R^{B} and R^{C} does not represent F.

According to one embodiment, W represents CH.

According to one embodiment, at least one among R^{A}, R^{B}, R^{C} and R^{D} represents hydrogen. In one embodiment, exactly one among R^{A}, R^{B}, R^{C} and R^{D} represents hydrogen. In one embodiment, exactly two among R^{A}, R^{B}, R^{C} and R^{D} represents hydrogen. In one embodiment, exactly three among R^{A}, R^{B}, R^{C} and R^{D} represents hydrogen.

In one embodiment, at least one among R^{A} and R^{C} represents hydrogen. In one particular embodiment, R^{A} represents hydrogen. In one particular embodiment, R^{C} represents hydrogen.

According to one embodiment, at least one among R^{A}, R^{B} and R^{D} represents F or Cl. In one embodiment, at least one among R^{B} and R^{D} represents F or Cl. In one particular embodiment, R^{B} and R^{D} each independently represents F or Cl. In one particular embodiment, R^{B} represents F. In one particular embodiment, R^{B} represents Cl. In one particular embodiment, R^{D} represents F. In one particular embodiment, R^{D} represents Cl. In one preferred embodiment, R^{B} represents F. In one preferred embodiment, R^{D} represents Cl. In one further preferred embodiment, R^{B} represents F and R^{D} represents Cl.

According to one embodiment, at least one among R^{A}, R^{B} and R^{D} represents CHF₂ or CF₃. In one embodiment, at least one among R^{B} and R^{D} represents CHF₂ or CF₃. In one particular embodiment, R^{B} and R^{D} each independently represents H, F, CHF₂ or CF₃. In one embodiment, R^{B} represents H or F. In one further preferred embodiment, R^{B} represents F. In one preferred embodiment, R^{D} represents CHF₂ or CF₃. In one further preferred embodiment, R^{B} represents H or F and R^{D} represents CHF₂ or CF₃.

According to one embodiment, R⁷ represents hydrogen, OH, halogen, (C₁-C₈) alkyl, cycloalkyl, (C₁-C₈) alkyl-O—, cycloalkyl-O—, cycloalkyl-(C₁-C₈) alkyl-O—, heterocycloalkyl-O—, R¹¹O—(C₁-C₈) alkyl-O—, R¹¹R¹²N—(C₁-C₈) alkyl-O— or (R¹¹O)(R¹²)N—(C₁-C₈) alkyl-O—; wherein R¹¹ and R¹² each independently represents hydrogen or (C₁-C₈) alkyl; wherein the alkyl, cycloalkyl, heterocycloalkyl, aryl or heteroaryl is optionally substituted as defined under formula (I) hereinabove.

According to one embodiment, R⁷ represents hydrogen, OH or halogen. In one embodiment, R⁷ represents hydrogen. In one preferred embodiment, R⁷ represents hydroxyl (OH). In one embodiment, R⁷ represents halogen. In one particular preferred embodiment, R⁷ represents F or Cl. In one particular preferred embodiment, R⁷ represents F. In one particular embodiment, R⁷ represents Cl.

In one preferred embodiment, R⁷ does not represent hydrogen.

According to one embodiment, R⁷ represents (C₁-C₈) alkyl-O— (i.e., (C₁-C₈) alkoxy) or cycloalkyl-O—. In one embodiment, the alkyl or cycloalkyl is optionally substituted by at least one F, Cl, OH, (C₁-C₈) alkoxy or aryl. In one particular embodiment, R⁷ represents OCH₃ (methoxy), OCH₂CH₃, OCF₃, cyclobutyl-O—, HO—CH₂—CH₂—O—, CH₃O—CH₂—CH₂—O— or phenyl-CH₂—O—.

According to one embodiment, R⁷ represents (C₁-C₈) alkyl-O-(i.e., (C₁-C₈) alkoxy). In one embodiment, the alkyl is optionally substituted by at least one F or Cl. In one particular embodiment, the halogen is F. In one embodiment, R⁷ represents OCH₃ (methoxy), OCH₂CH₃ or OCF₃. In one preferred embodiment, R⁷ represents OCH₃ (methoxy).

According to one embodiment, R⁷ represents cycloalkyl-O—. In one embodiment, R⁷ represents cyclobutyl-O—.

According to one embodiment, R⁷ represents (C₁-C₈) alkyl-O-(i.e., (C₁-C₈) alkoxy), wherein the alkyl is optionally substituted by at least one OH or (C₁-C₈) alkoxy, i.e., RO—(C₁-C₈) alkyl-O— wherein R represents H or (C₁-C₈) alkyl. In one preferred embodiment, R⁷ represents HO—CH₂—CH₂—O— or CH₃O—CH₂—CH₂—O—.

According to one embodiment, R⁷ represents (C₁-C₈) alkyl-O— (i.e., (C₁-C₈) alkoxy), wherein the alkyl is optionally substituted by at least one aryl. In one embodiment, the aryl is not substituted. In one embodiment, R⁷ represents phenyl-CH₂—O—.

According to one embodiment, R⁷ represents (C₁-C₈) alkyl-O— (i.e., (C₁-C₈) alkoxy), wherein the alkyl is optionally substituted by at least one heteroaryl. In one embodiment, the heteroaryl is not substituted. In one embodiment, R⁷ represents heteroaryl-CH₂—O—.

According to one embodiment, R⁷ represents (C₁-C₈) alkyl-O-(i.e., (C₁-C₈) alkoxy), wherein the alkyl is optionally substituted by at least one heterocycloalkyl. In one embodiment, R⁷ represents heterocycloalkyl-(C₁-C₈) alkyl-O—, i.e., the alkyl is substituted by exactly one heterocycloalkyl. In one embodiment, the heterocycloalkyl is not substituted.

In one preferred embodiment, R⁷ represents hydrogen, F, Cl, OH, OCH₃ (methoxy), HO—CH₂—CH₂—O—, CH₃O—CH₂—CH₂—O— or phenyl-CH₂—O—.

In one further preferred embodiment, R⁷ represents F, OH, OCH₃ (methoxy) or phenyl-CH₂—O—.

According to one embodiment, Z represents C—H or N; or Z represents C—R⁸ and R⁷ and R⁸ form together with the carbon atoms to which they are bound a cycloalkyl or heterocycloalkyl; wherein the cycloalkyl or heterocycloalkyl is optionally substituted as defined under formula (I) hereinabove. In other words, in this embodiment, R⁸ represents hydrogen, except where R⁷ and R⁸ form together with the carbon atoms to which they are bound a cycloalkyl or heterocycloalkyl.

According to one embodiment, Z represents C—H or N. In one embodiment, Z represents C—H. In one embodiment, Z represents N.

According to another embodiment, Z represents C—R⁸ and R⁷ and R⁸ form together with the carbon atoms to which they are bound a cycloalkyl or heterocycloalkyl; wherein the cycloalkyl or heterocycloalkyl is optionally substituted as defined under formula (I) herein.

According to one preferred embodiment, Z does not represent N.

In one preferred embodiment, Z represents C—R⁸; wherein R⁸ represents (C₁-C₄) alkyl, F, Cl, CF₃, CHF₂, CH₂F, OCF₃, CN, OH or (C₁-C₄) alkoxy. In one particular embodiment, R⁸ represents methyl, ethyl, F, Cl, CF₃, CN or OH. In one preferred embodiment, R⁸ represents methyl or Cl.

In one preferred embodiment, Z represents C—R⁸ and R⁷ and R⁸ form together with the carbon atoms to which they are bound an heterocycloalkyl; wherein the heterocycloalkyl is optionally substituted as defined under formula (I) herein. In one particular embodiment, the heterocycloalkyl comprises at least one oxygen atom, i.e., the heterocycloalkyl is a cyclic ether. In one particular embodiment, the heterocycloalkyl comprises exactly one oxygen atom. In one particular embodiment, the heterocycloalkyl comprises at least one nitrogen atom, i.e., the heterocycloalkyl is a cyclic amine. In one particular embodiment, the heterocycloalkyl is six-membered or five-membered. In one particular embodiment, the heterocycloalkyl is not substituted. In one preferred embodiment, the heterocycloalkyl is tetrahydrofuran (e.g., wherein —R⁷-R⁸— represents —O—CH₂CH₂—).

According to one embodiment, R⁵ represents hydrogen or (C₁-C₈) alkyl. In one embodiment, R⁵ represents hydrogen. In one embodiment, R⁵ represents (C₁-C₈) alkyl. In one preferred embodiment, R⁵ represents hydrogen, methyl or ethyl. In one particular embodiment, R⁵ represents hydrogen. In one particular embodiment, R⁵ represents methyl or ethyl. In one further particular embodiment, R⁵ represents methyl. In one further particular embodiment, R⁵ represents ethyl.

According to one embodiment, R⁶ represents (C₁-C₈) alkyl, cycloalkyl or cycloalkyl-(C₁-C₈) alkyl-. In one embodiment, the alkyl or cycloalkyl is optionally substituted by at least one F. In one embodiment, the alkyl or cycloalkyl is not substituted. In one embodiment, R⁶ represents methyl, ethyl, n-propyl, 1-fluoro-n-propane, tert-butyl, cyclopropyl, cyclobutyl or cyclopropyl-CH₂—.

According to one embodiment, R⁶ represents (C₁-C₈) alkyl, cycloalkyl, heterocycloalkyl, cycloalkyl-(C₁-C₈) alkyl- or aryl-(C₁-C₈) alkyl-. In one embodiment, the alkyl, cycloalkyl, heterocycloalkyl or aryl is optionally substituted by at least one methyl, Cl or F.

In one embodiment, R⁶ represents ethyl, propyl (e.g., n-propyl), butyl (e.g., tert-butyl), cyclopentyl, cyclohexyl, 2-adamantyl, 3-methyloxetan-3-yl, cyclopropyl-CH₂—, cyclobutyl-CH₂—, cyclohexyl-CH₂—, phenyl-CH₂— (benzyl), 3-chlorophenyl-CH₂— or 4-fluorophenyl-CH₂—.

In one further preferred embodiment, R⁶ represents propyl (e.g., n-propyl), butyl (e.g., tert-butyl), cyclohexyl, cyclopropyl-CH₂— or 3-chlorophenyl-CH₂—.

According to one embodiment, R⁶ represents (C₁-C₈) alkyl. In one embodiment, the alkyl is optionally substituted by at least one F. In one embodiment, the alkyl is not substituted. In one particular embodiment, R⁵ represents methyl, ethyl, n-propyl, 1-fluoro-n-propane or tert-butyl.

According to one embodiment, R⁶ represents cycloalkyl. In one embodiment, R⁶ represents cyclopropyl or cyclobutyl.

According to one embodiment, R⁶ represents cycloalkyl-(C₁-C₈) alkyl. In one particular embodiment, R⁶ represents cyclopropylmethyl (cyclopropyl-CH₂—).

According to one embodiment, R⁵ and R⁶ form together with the nitrogen atom to which they are bound an heterocycloalkyl. In one embodiment, the heterocycloalkyl is optionally substituted by at least one F. In one embodiment, R⁵ and R⁶ form together with the nitrogen atom to which they are bound a pyrrolidine, piperidine, 4-fluoropiperidine, 3-fluoropyrrolidine, 4-trifluoromethylpiperidine, 4-benzyl-piperidine, 3-benzylpyrrolidine, 3-benzyl-piperidine, 4-phenylpiperidine, 4-benzylidenepiperidine, octahydro-1H-isoindole, 2-benzyloctahydropyrrolo[3,4-c]pyrrole, 3-(benzyloxy)pyrrole, 3-(methoxymethyl)azetidine, 2-azabicyclo[2.2.1]heptane, 5-(4-methylpiperazin-1-yl)pyrimidine and 4-phenethylpiperidine.

In one particular embodiment, R⁵ and R⁶ form together with the nitrogen atom to which they are bound a pyrrolidine, 4-fluoropiperidine, 3-fluoropyrrolidine, 4-trifluoromethylpiperidine, 4-benzyl-piperidine, 3-benzylpyrrolidine, 3-benzyl-piperidine, 4-phenylpiperidine, 4-benzylidenepiperidine, octahydro-1H-isoindole, 3-(benzyloxy)pyrrole, 2-azabicyclo[2.2.1]heptane, 5-(4-methylpiperazin-1-yl)pyrimidine or 4-phenethylpiperidine.

In one preferred embodiment, R⁵ and R⁶ form together with the nitrogen atom to which they are bound a pyrrolidine, 4-benzyl-piperidine, 4-phenyl-4-hydroxy-piperidine, 4-benzyl-4-hydroxy-piperidine, 4-benzyl-piperazine, tert-butyl 1,3,3a,4,6,6a-hexahydropyrrolo[3,4-c]pyrrole-2-carboxylate, 4-benzylidene-1-piperidine, 4-(2-phenylethyl)-piperidine, 4-phenylpiperidine, 3-phenylpiperidine, 3-benzyl-piperidine, 3-phenylpyrrolidin, 3-benzylpyrrolidine, 4-trifluoromethylpiperidine, 2-azabicyclo[2.2.1]heptane, 3-benzyloxypiperidine, 3-benzyloxypyrrolidine, 2-benzyl-1,3,3a,4,6,6a-hexahydropyrrolo[3,4-c]pyrrolidine, 4-(4-fluorophenyl)piperidine, [2-(4-chlorophenyl)ethyl]piperazine, [2-(4-fluorophenyl)ethyl]piperazine, 2-(phenylpropyl)piperazine, [2-(4-fluorophenyl)propyl]piperazine, 4-fluoropiperidine or 2,2-dimethylpyrrolidine.

In one preferred embodiment, R⁵ and R⁶ form together with the nitrogen atom to which they are bound a pyrrolidine, 4-benzyl-piperidine, 4-benzylidene-1-piperidine, 4-(2-phenylethyl)-piperidine, 4-phenylpiperidine, 3-phenylpiperidine, 3-benzyl-piperidine, 3-phenylpyrrolidin, 4-trifluoromethylpiperidine, 3-benzyloxypiperidine, 3-benzyloxypyrrolidine, 4-(4-fluorophenyl)piperidine, [2-(4-chlorophenyl)ethyl]piperazine, [2-(4-fluorophenyl)ethyl]piperazine, 2-(phenylpropyl)piperazine, [2-(4-fluorophenyl)propyl]piperazine, 4-fluoropiperidine or2,2-dimethylpyrrolidine.

According to one embodiment, at least one heterocycloalkyl present in the compound of formula (I) is a water-solubilizing group, i.e., the presence of this group in the molecule increases the solubility thereof in water, compared with the same molecule not comprising the heterocycloalkyl.

According to one embodiment, the compound of formula (I) is a compound of formula (I-a)

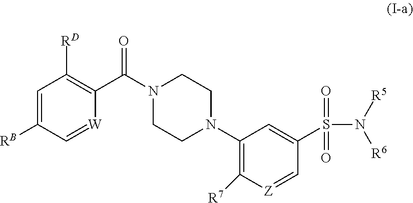

or a pharmaceutically acceptable salt and/or solvate thereof;
   wherein W, R^{B}, R^{D}, Z and R⁵-R⁷ are as defined under formula (I) herein.

In one embodiment, W in formula (I-a) represents CH.

In one embodiment, R^{b} and R^{D} in formula (I-a) each independently represents F or Cl, and R⁷ represents alkoxy. In one particular embodiment, R^{b} represents F and R^{D} represents Cl.

In one embodiment, R⁷ in formula (I-a) represents alkoxy. In one particular embodiment, R⁷ represents methoxy (—OCH₃).

In one embodiment, R^{b} and R^{D} in formula (I-a) each independently represents F or CF₃. In one particular embodiment, R^{b} represents F and R^{D} represents CF₃.

In one embodiment, Z in formula (I-a) represents N.

According to one embodiment, the compound of formula (I) is selected from the compounds of Table 1 below, and pharmaceutically acceptable salts and/or solvates thereof.

TABLE 1

| Cpd | Structure | Name |
|---|---|---|
| 001 | 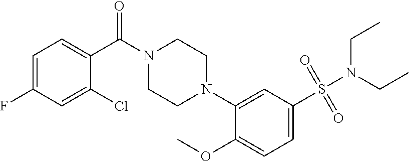 [SMILES: CCN(CC)S(=O)(=O)c1ccc(OC)c(N2CCN(C(=O)c3ccc(F)cc3Cl)CC2)c1] | 3-[4-(2-chloro-4- fluoro- benzoyl)piperazin-1- yl]-N,N-diethyl-4- methoxy- benzenesulfonamide |
| 002 | 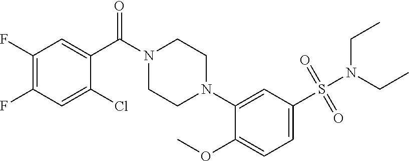 [SMILES: CCN(CC)S(=O)(=O)c1ccc(OC)c(N2CCN(C(=O)c3cc(F)c(F)cc3Cl)CC2)c1] | 3-[4-(2-chloro-4,5- difluoro- benzoyl)piperazin-1- yl]-N,N-diethyl-4- methoxy- benzenesulfonamide |
| 003 | 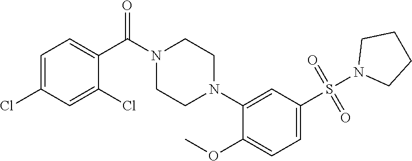 [SMILES: COc1ccc(S(=O)(=O)N2CCCC2)cc1N1CCN(C(=O)c2ccc(Cl)cc2Cl)CC1] | (2,4-dichlorophenyl)- [4-(2-methoxy-5- pyrrolidin-1- ylsulfonyl- phenyl)piperazin-1- yl]methanone |
| 004 | 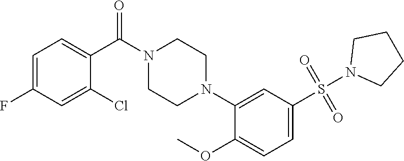 [SMILES: COc1ccc(S(=O)(=O)N2CCCC2)cc1N1CCN(C(=O)c2ccc(F)cc2Cl)CC1] | (2-chloro-4-fluoro- phenyl)-[4-(2- methoxy-5-pyrrolidin- 1-ylsulfonyl- phenyl)piperazin-1- yl]methanone |
| 005 | 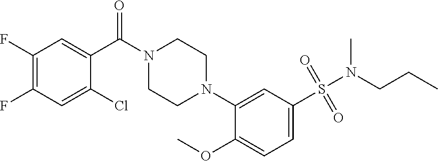 [SMILES: CCCN(C)S(=O)(=O)c1ccc(OC)c(N2CCN(C(=O)c3cc(F)c(F)cc3Cl)CC2)c1] | 3-[4-(2-chloro-4,5- difluoro- benzoyl)piperazin-1- yl]-4-methoxy-N- methyl-N-propyl- benzenesulfonamide |
| 006 | 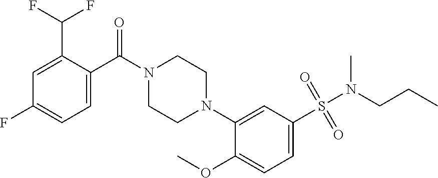 [SMILES: CCCN(C)S(=O)(=O)c1ccc(OC)c(N2CCN(C(=O)c3ccc(F)cc3C(F)F)CC2)c1] | 3-[4-[2- (difluoromethyl)-4- fluoro- benzoyl]piperazin-1- yl]-4-methoxy-N- methyl-N-propyl- benzenesulfonamide |
| 007 | 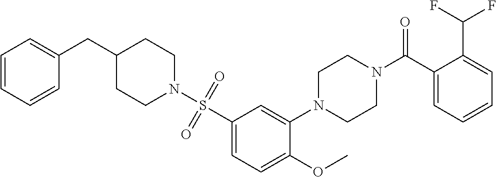 [SMILES: COc1ccc(S(=O)(=O)N2CCC(Cc3ccccc3)CC2)cc1N1CCN(C(=O)c2ccccc2C(F)F)CC1] | [4-[5-[(4-benzyl-1- piperidyl)sulfonyl]-2- methoxy- phenyl]piperazin-1-yl]- [2- (difluoromethyl)phenyl] methanone |
| 008 | 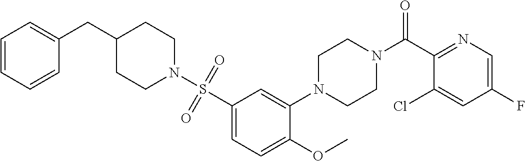 [SMILES: COc1ccc(S(=O)(=O)N2CCC(Cc3ccccc3)CC2)cc1N1CCN(C(=O)c2ncc(F)cc2Cl)CC1] | [4-[5-[(4-benzyl-1- piperidyl)sulfonyl]-2- methoxy- phenyl]piperazin-1-yl]- (3-chloro-5-fluoro-2- pyridyl)methanone |
| 009 | 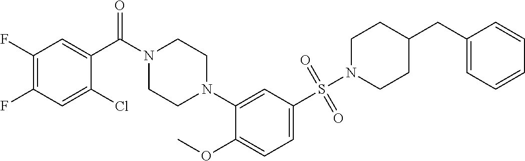 [SMILES: COc1ccc(S(=O)(=O)N2CCC(Cc3ccccc3)CC2)cc1N1CCN(C(=O)c2cc(F)c(F)cc2Cl)CC1] | [4-[5-[(4-benzyl-1- piperidyl)sulfonyl]-2- methoxy- phenyl]piperazin-1-yl]- (2-chloro-4,5-difluoro- phenyl)methanone |
| 010 | 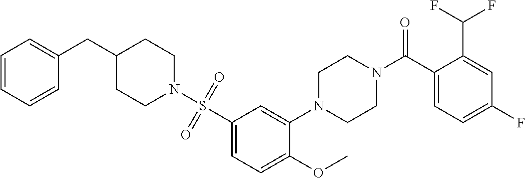 [SMILES: COc1ccc(S(=O)(=O)N2CCC(Cc3ccccc3)CC2)cc1N1CCN(C(=O)c2ccc(F)cc2C(F)F)CC1] | [4-[5-[(4-benzyl-1- piperidyl)sulfonyl]-2- methoxy- phenyl]piperazin-1-yl]- [2-(difluoromethyl)-4- fluoro- phenyl]methanone |
| 011 | 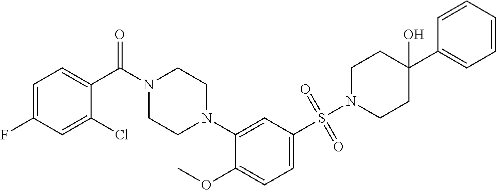 [SMILES: COc1ccc(S(=O)(=O)N2CCC(O)(c3ccccc3)CC2)cc1N1CCN(C(=O)c2ccc(F)cc2Cl)CC1] | (2-chloro-4-fluoro- phenyl)-[4-[5-[(4- hydroxy-4-phenyl-1- piperidyl)sulfonyl]-2- methoxy- phenyl]piperazin-1- yl]methanone |
| 012 | 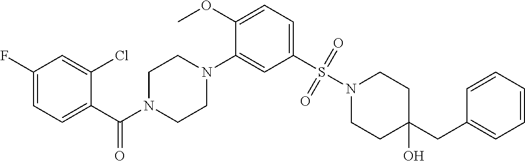 [SMILES: COc1ccc(S(=O)(=O)N2CCC(O)(Cc3ccccc3)CC2)cc1N1CCN(C(=O)c2ccc(F)cc2Cl)CC1] | [4-[5-[(4-benzyl-4- hydroxy-1- piperidyl)sulfonyl]-2- methoxy- phenyl]piperazin-1-yl]- (2-chloro-4-fluoro- phenyl)methanone |
| 013 | 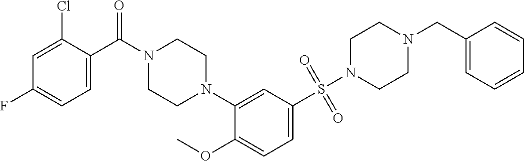 [SMILES: COc1ccc(S(=O)(=O)N2CCN(Cc3ccccc3)CC2)cc1N1CCN(C(=O)c2ccc(F)cc2Cl)CC1] | [4-[5-(4- benzylpiperazin-1- yl)sulfonyl-2-methoxy- phenyl]piperazin-1-yl]- (2-chloro-4-fluoro- phenyl)methanone |
| 014 | 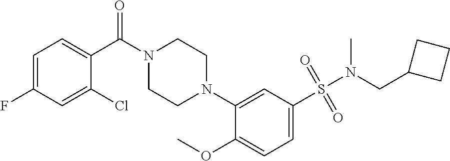 [SMILES: COc1ccc(S(=O)(=O)N(C)CC2CCC2)cc1N1CCN(C(=O)c2ccc(F)cc2Cl)CC1] | 3-[4-(2-chloro-4- fluoro- benzoyl)piperazin-1- yl]-N- (cyclobutylmethyl)-4- methoxy-N-methyl- benzenesulfonamide |
| 015 | 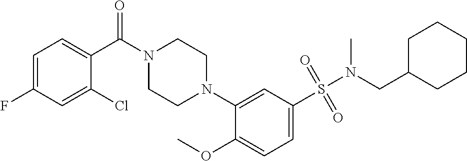 [SMILES: COc1ccc(S(=O)(=O)N(C)CC2CCCCC2)cc1N1CCN(C(=O)c2ccc(F)cc2Cl)CC1] | 3-[4-(2-chloro-4- fluoro- benzoyl)piperazin-1- yl]-N- (cyclohexylmethyl)-4- methoxy-N-methyl- benzenesulfonamide |
| 016 | 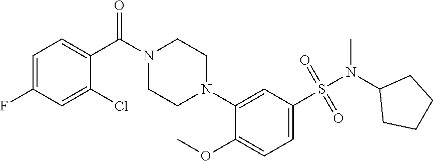 [SMILES: COc1ccc(S(=O)(=O)N(C)C2CCCC2)cc1N1CCN(C(=O)c2ccc(F)cc2Cl)CC1] | 3-[4-(2-chloro-4- fluoro- benzoyl)piperazin-1- yl]-N-cyclopentyl-4- methoxy-N-methyl- benzenesulfonamide |
| 017 | 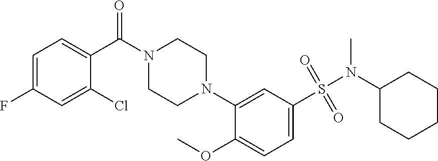 [SMILES: COc1ccc(S(=O)(=O)N(C)C2CCCCC2)cc1N1CCN(C(=O)c2ccc(F)cc2Cl)CC1] | 3-[4-(2-chloro-4- fluoro- benzoyl)piperazin-1- yl]-N-cyclohexyl-4- methoxy-N-methyl- benzenesulfonamide |
| 018 | 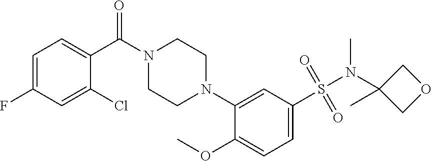 [SMILES: COc1ccc(S(=O)(=O)N(C)C2(C)COC2)cc1N1CCN(C(=O)c2ccc(F)cc2Cl)CC1] | 3-[4-(2-chloro-4- fluoro- benzoyl)piperazin-1- yl]-4-methoxy-N- methyl-N-(3- methyloxetan-3- yl)benzenesulfonamide |
| 019 | 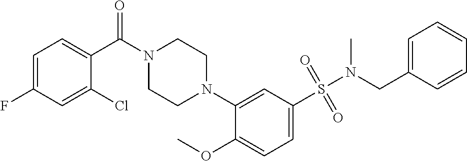 [SMILES: COc1ccc(S(=O)(=O)N(C)Cc2ccccc2)cc1N1CCN(C(=O)c2ccc(F)cc2Cl)CC1] | N-benzyl-3-[4-(2- chloro-4-fluoro- benzoyl)piperazin-1- yl]-4-methoxy-N- methyl- benzenesulfonamide |
| 020 | 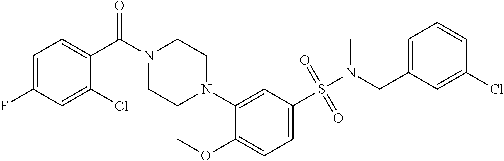 [SMILES: COc1ccc(S(=O)(=O)N(C)Cc2cccc(Cl)c2)cc1N1CCN(C(=O)c2ccc(F)cc2Cl)CC1] | 3-[4-(2-chloro-4- fluoro- benzoyl)piperazin-1- yl]-N-[(3- chlorophenyl)methyl]- 4-methoxy-N-methyl- benzenesulfonamide |
| 021 | 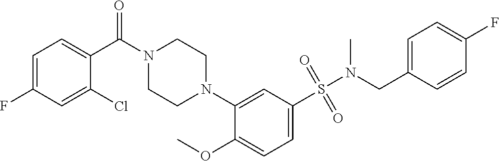 [SMILES: COc1ccc(S(=O)(=O)N(C)Cc2ccc(F)cc2)cc1N1CCN(C(=O)c2ccc(F)cc2Cl)CC1] | 3-[4-(2-chloro-4- fluoro- benzoyl)piperazin-1- yl]-N-[(4- fluorophenyl)methyl]- 4-methoxy-N-methyl- benzenesulfonamide |
| 022 | 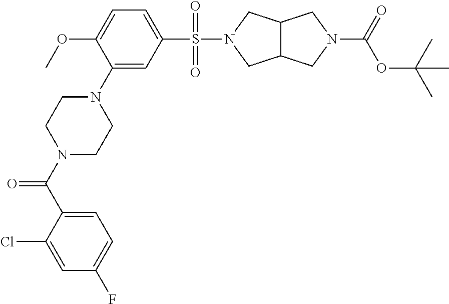 [SMILES: COc1ccc(S(=O)(=O)N2CC3CN(C(=O)OC(C)(C)C)CC3C2)cc1N1CCN(C(=O)c2ccc(F)cc2Cl)CC1] | tert-butyl 5-[3-[4-(2- chloro-4-fluoro- benzoyl)piperazin-1- yl]-4-methoxy- phenyl]sulfonyl- 1,3,3a,4,6,6a- hexahydropyrrolo[3,4- c]pyrrole-2- carboxylate |
| 023 | 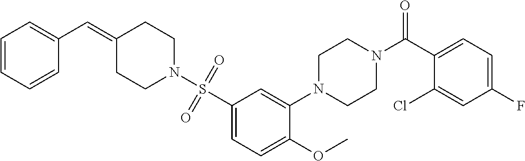 [SMILES: COc1ccc(S(=O)(=O)N2CCC(=Cc3ccccc3)CC2)cc1N1CCN(C(=O)c2ccc(F)cc2Cl)CC1] | [4-[5-[(4-benzylidene- 1-piperidyl)sulfonyl]- 2-methoxy- phenyl]piperazin-1-yl]- (2-chloro-4-fluoro- phenyl)methanone |
| 024 | 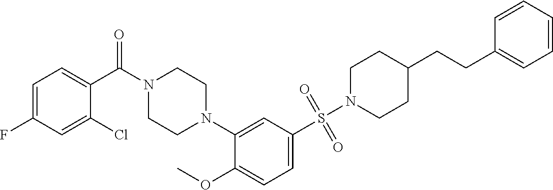 [SMILES: COc1ccc(S(=O)(=O)N2CCC(CCc3ccccc3)CC2)cc1N1CCN(C(=O)c2ccc(F)cc2Cl)CC1] | (2-chloro-4-fluoro- phenyl)-[4-[2- methoxy-5-[[4-(2- phenylethyl)-1- piperidyl]sulfonyl] phenyl]piperazin-1- yl]methanone |
| 025 | 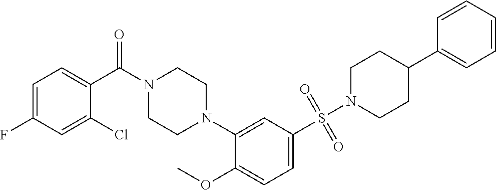 [SMILES: COc1ccc(S(=O)(=O)N2CCC(c3ccccc3)CC2)cc1N1CCN(C(=O)c2ccc(F)cc2Cl)CC1] | (2-chloro-4-fluoro- phenyl)-[4-[2- methoxy-5-[(4-phenyl- 1- piperidyl)sulfonyl] phenyl]piperazin-1- yl]methanone |
| 026 | 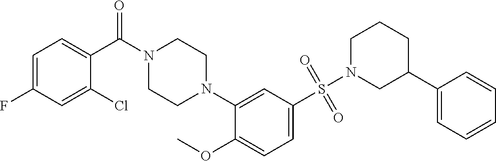 [SMILES: COc1ccc(S(=O)(=O)N2CCCC(c3ccccc3)C2)cc1N1CCN(C(=O)c2ccc(F)cc2Cl)CC1] | (2-chloro-4-fluoro- phenyl)-[4-[2- methoxy-5-[3-phenyl- 1- piperidyl]sulfonyl] phenyl]piperazin-1- yl]methanone |
| 027 | 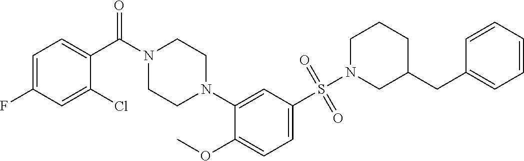 [SMILES: COc1ccc(S(=O)(=O)N2CCCC(Cc3ccccc3)C2)cc1N1CCN(C(=O)c2ccc(F)cc2Cl)CC1] | (2-chloro-4-fluoro- phenyl)-[4-[2- methoxy-5-[[3-benzyl- 1- piperidyl]sulfonyl] phenyl]piperazin-1- yl]methanone |
| 028 | 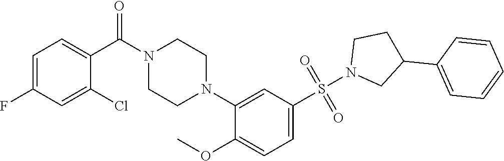 [SMILES: COc1ccc(S(=O)(=O)N2CCC(c3ccccc3)C2)cc1N1CCN(C(=O)c2ccc(F)cc2Cl)CC1] | (2-chloro-4-fluoro- phenyl)-[4-[2- methoxy-5-[3- phenylpyrrolidin-1- yl]sulfonyl- phenyl]piperazin-1- yl]methanone |
| 029 | 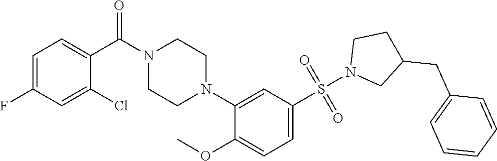 [SMILES: COc1ccc(S(=O)(=O)N2CCC(Cc3ccccc3)C2)cc1N1CCN(C(=O)c2ccc(F)cc2Cl)CC1] | (2-chloro-4-fluoro- phenyl)-[4-[2- methoxy-5-[3- benzylpyrrolidin-1- yl]sulfonyl- phenyl]piperazin-1- yl]methanone |
| 030 | 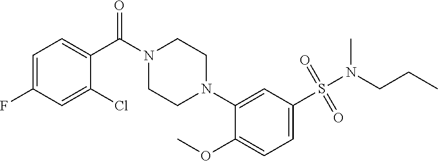 [SMILES: CCCN(C)S(=O)(=O)c1ccc(OC)c(N2CCN(C(=O)c3ccc(F)cc3Cl)CC2)c1] | 3-[4-(2-chloro-4- fluoro- benzoyl)piperazin-1- yl]-4-methoxy-N- methyl-N-propyl- benzenesulfonamide |
| 031 | 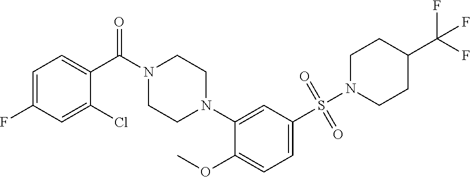 [SMILES: COc1ccc(S(=O)(=O)N2CCC(C(F)(F)F)CC2)cc1N1CCN(C(=O)c2ccc(F)cc2Cl)CC1] | (2-chloro-4-fluoro- phenyl)-[4-[2- methoxy-5-[[4- (trifluoromethyl)-1- piperidyl]sulfonyl] phenyl]piperazin-1- yl]methanone |
| 032 | 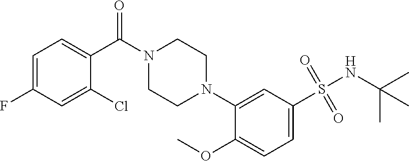 [SMILES: COc1ccc(S(=O)(=O)NC(C)(C)C)cc1N1CCN(C(=O)c2ccc(F)cc2Cl)CC1] | N-tert-butyl-3-[4-(2- chloro-4-fluoro- benzoyl)piperazin-1- yl]-4-methoxy- benzenesulfonamide |
| 033 | 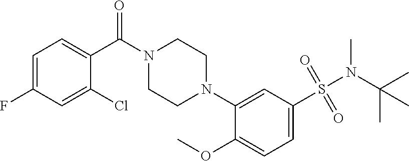 [SMILES: COc1ccc(S(=O)(=O)N(C)C(C)(C)C)cc1N1CCN(C(=O)c2ccc(F)cc2Cl)CC1] | N-tert-butyl-3-[4-(2- chloro-4-fluoro- benzoyl)piperazin-1- yl]-4-methoxy-N- methyl- benzenesulfonamide |
| 034 | 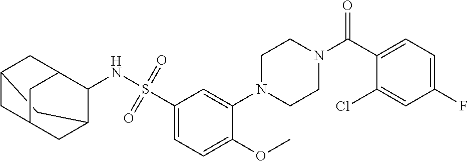 [SMILES: COc1ccc(S(=O)(=O)NC2C3CC4CC(C3)CC2C4)cc1N1CCN(C(=O)c2ccc(F)cc2Cl)CC1] | N-(2-adamantyl)-3-[4- (2-chloro-4-fluoro- benzoyl)piperazin-1- yl]-4-methoxy- benzenesulfonamide |
| 035 | 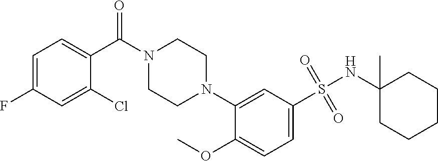 [SMILES: COc1ccc(S(=O)(=O)NC2(C)CCCCC2)cc1N1CCN(C(=O)c2ccc(F)cc2Cl)CC1] | 3-[4-(2-chloro-4- fluoro- benzoyl)piperazin-1- yl]-4-methoxy-N-(1- methylcyclohexyl) benzenesulfonamide |
| 036 | 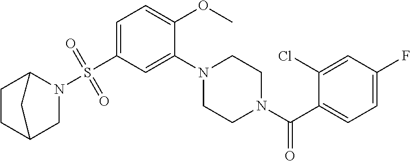 [SMILES: COc1ccc(S(=O)(=O)N2CC3CCC2C3)cc1N1CCN(C(=O)c2ccc(F)cc2Cl)CC1] | (2-chloro-4-fluoro- phenyl)-[4-[2- methoxy-5-[[2- azabicyclo[2.2.1 ] heptan-2- yl]sulfonyl]phenyl] piperazin-1-yl] methanone |
| 037 | 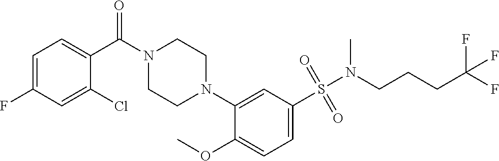 [SMILES: COc1ccc(S(=O)(=O)N(C)CCCC(F)(F)F)cc1N1CCN(C(=O)c2ccc(F)cc2Cl)CC1] | 3-[4-(2-chloro-4- fluoro- benzoyl)piperazin-1- yl]-4-methoxy-N- methyl-N-(4,4,4- trifluorobutyl) benzenesulfonamide |
| 038 | 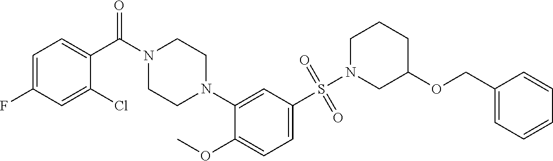 [SMILES: COc1ccc(S(=O)(=O)N2CCCC(OCc3ccccc3)C2)cc1N1CCN(C(=O)c2ccc(F)cc2Cl)CC1] | (2-chloro-4-fluoro- phenyl)-[4-[2- methoxy-5-[[3- benzyloxy-1- piperidyl]sulfonyl] phenyl]piperazin-1- yl]methanone |
| 039 | 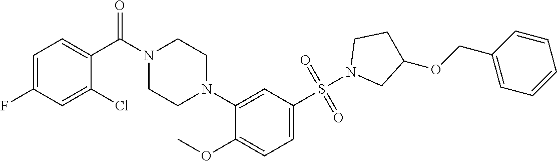 [SMILES: COc1ccc(S(=O)(=O)N2CCC(OCc3ccccc3)C2)cc1N1CCN(C(=O)c2ccc(F)cc2Cl)CC1] | (2-chloro-4-fluoro- phenyl)-[4-[2- methoxy-5-[3- benzyloxypyrrolidin-1- yl]sulfonyl- phenyl]piperazin-1- yl]methanone |
| 040 | 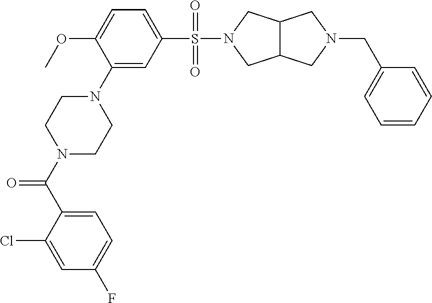 [SMILES: COc1ccc(S(=O)(=O)N2CC3CN(Cc4ccccc4)CC3C2)cc1N1CCN(C(=O)c2ccc(F)cc2Cl)CC1] | [4-[5-[[2-benzyl- 1,3,3a,4,6,6a- hexahydropyrrolo[3,4- c]pyrrol-5-yl]sulfonyl]- 2-methoxy- phenyl]piperazin-1-yl]- (2-chloro-4-fluoro- phenyl)methanone |
| 041 | 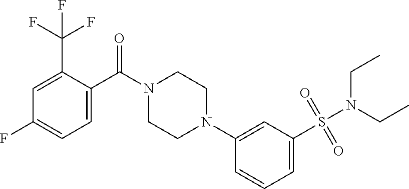 [SMILES: CCN(CC)S(=O)(=O)c1cccc(N2CCN(C(=O)c3ccc(F)cc3C(F)(F)F)CC2)c1] | N,N-diethyl-3-[4-[4- fluoro-2- (trifluoromethyl)benzoyl] piperazin-1- yl]benzenesulfonamide |
| 042 | 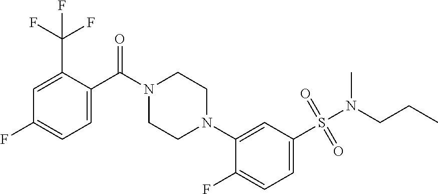 [SMILES: CCCN(C)S(=O)(=O)c1ccc(F)c(N2CCN(C(=O)c3ccc(F)cc3C(F)(F)F)CC2)c1] | 4-fluoro-3-[4-[4- fluoro-2- (trifluoromethyl)benzoyl] piperazin-1-yl]-N- methyl-N-propyl- benzenesulfonamide |
| 043 | 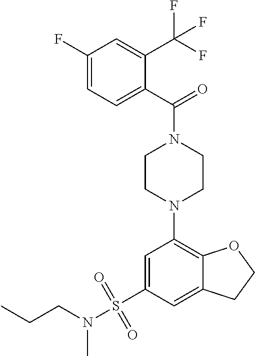 [SMILES: CCCN(C)S(=O)(=O)c1cc2c(c(N3CCN(C(=O)c4ccc(F)cc4C(F)(F)F)CC3)c1)OCC2] | 7-[4-[4-fluoro-2- (trifluoromethyl)benzoyl] piperazin-1-yl]-N- methyl-N-propyl-2,3- dihydrobenzofuran-5- sulfonamide |
| 044 | 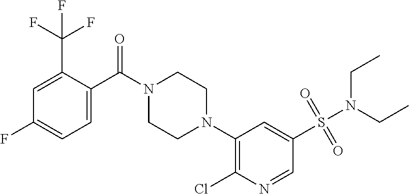 [SMILES: CCN(CC)S(=O)(=O)c1cnc(Cl)c(N2CCN(C(=O)c3ccc(F)cc3C(F)(F)F)CC2)c1] | 6-chloro-N,N-diethyl- 5-[4-[4-fluoro-2- (trifluoromethyl)benzoyl] piperazin-1- yl]pyridine-3- sulfonamide |
| 045 | 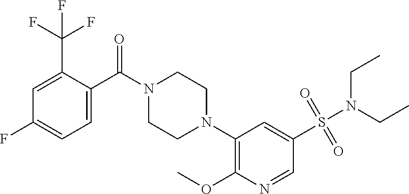 [SMILES: CCN(CC)S(=O)(=O)c1cnc(OC)c(N2CCN(C(=O)c3ccc(F)cc3C(F)(F)F)CC2)c1] | N,N-diethyl-5-[4-[4- fluoro-2- (trifluoromethyl)benzoyl] piperazin-1-yl]-6- methoxy-pyridine-3- sulfonamide |
| 046 | 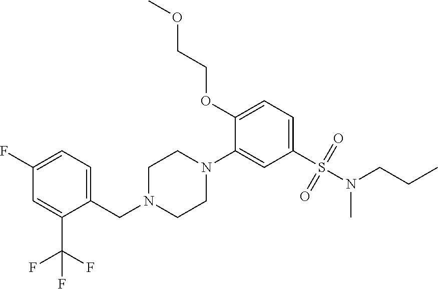 [SMILES: CCCN(C)S(=O)(=O)c1ccc(OCCOC)c(N2CCN(Cc3ccc(F)cc3C(F)(F)F)CC2)c1] | 3-[4-[4-fluoro-2- (trifluoromethyl)benzoyl] piperazin-1-yl]-4-(2- methoxyethoxy)-N- methyl-N-propyl- benzenesulfonamide |
| 047 | 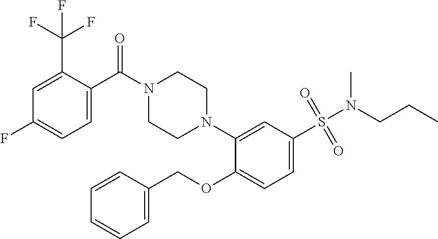 [SMILES: CCCN(C)S(=O)(=O)c1ccc(OCc2ccccc2)c(N2CCN(C(=O)c3ccc(F)cc3C(F)(F)F)CC2)c1] | 4-benzyloxy-3-[4-[4- fluoro-2- (trifluoromethyl)benzoyl] piperazin-1-yl]-N- methyl-N-propyl- benzenesulfonamide |
| 048 | 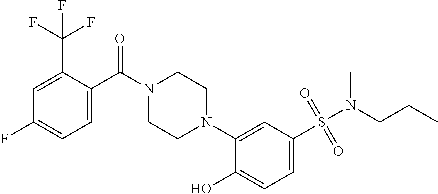 [SMILES: CCCN(C)S(=O)(=O)c1ccc(O)c(N2CCN(C(=O)c3ccc(F)cc3C(F)(F)F)CC2)c1] | 3-[4-[4-fluoro-2- (trifluoromethyl)benzoyl] piperazin-1-yl]-4- hydroxy-N-methyl-N- propyl- benzenesulfonamide |
| 049 | 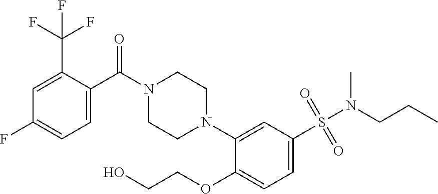 [SMILES: CCCN(C)S(=O)(=O)c1ccc(OCCO)c(N2CCN(C(=O)c3ccc(F)cc3C(F)(F)F)CC2)c1] | 3-[4-[4-fluoro-2- (trifluoromethyl)benzoyl] piperazin-1-yl]-4-(2- hydroxyethoxy)-N- methyl-N-propyl- benzenesulfonamide |
| 050 | 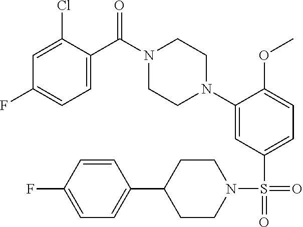 [SMILES: COc1ccc(S(=O)(=O)N2CCC(c3ccc(F)cc3)CC2)cc1N1CCN(C(=O)c2ccc(F)cc2Cl)CC1] | (2-chloro-4-fluoro- phenyl)-[4-[5-[[4-(4- fluorophenyl)-1- piperidyl]sulfonyl]-2- methoxy- phenyl]piperazin-1- yl]methanone |
| 051 | 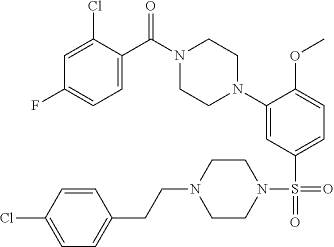 [SMILES: COc1ccc(S(=O)(=O)N2CCN(CCc3ccc(Cl)cc3)CC2)cc1N1CCN(C(=O)c2ccc(F)cc2Cl)CC1] | (2-chloro-4-fluoro- phenyl)-[4-[5-[4-[2-(4- chlorophenyl)ethyl] piperazin-1-yl]sulfonyl-2- methoxy- phenyl]piperazin-1- yl]methanone |
| 052 | 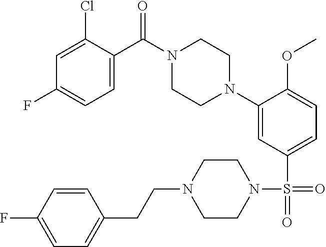 [SMILES: COc1ccc(S(=O)(=O)N2CCN(CCc3ccc(F)cc3)CC2)cc1N1CCN(C(=O)c2ccc(F)cc2Cl)CC1] | (2-chloro-4-fluoro- phenyl)-[4-[5-[4-[2-(4- fluorophenyl)ethyl] piperazin-1-yl]sulfonyl-2- methoxy- phenyl]piperazin-1- yl]methanone |
| 053 | 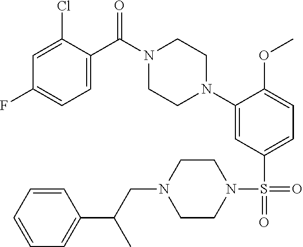 [SMILES: COc1ccc(S(=O)(=O)N2CCN(CC(C)c3ccccc3)CC2)cc1N1CCN(C(=O)c2ccc(F)cc2Cl)CC1] | (2-chloro-4-fluoro- phenyl)-[4-[2- methoxy-5-[4-[rac- (2R)-2- phenylpropyl]piperazin- 1-yl]sulfonyl- phenyl]piperazin-1- yl]methanone |
| 054 | | (2-chloro-4-fluoro- |
| | | phenyl)-[4-[2- |
| | | methoxy-5-[4-[rac- |
| | | (2R)-2-(4- |
| | | fluorophenyl)propyl] |
| | | piperazin-1-yl]sulfonyl- |
| | | phenyl]piperazin-1- |
| | | yl]methanone |
| 055 | | (2-chloro-4-fluoro- |
| | | phenyl)-[4-[2-hydroxy- |
| | | 3-methyl-5-[(4-phenyl- |
| | | 1- |
| | | piperidyl)sulfonyl] |
| | | phenyl]piperazin-1- |
| | | yl]methanone |
| 056 | | (2-chloro-4-fluoro- |
| | | phenyl)-[4-[5-[[4-(4- |
| | | fluorophenyl)-1- |
| | | piperidyl]sulfonyl]-2- |
| | | hydroxy-3-methyl- |
| | | phenyl]piperazin-1- |
| | | yl]methanone |
| 057 | | (2-chloro-4-fluoro- |
| | | phenyl)-[4-[3-chloro-5- |
| | | [(4-fluoro-1- |
| | | piperidyl)sulfonyl]-2- |
| | | hydroxy- |
| | | phenyl]piperazin-1- |
| | | yl]methanone |
| 058 | | N-tert-butyl-3-chloro- |
| | | 5-[4-(2-chloro-4- |
| | | fluoro- |
| | | benzoyl)piperazin-1- |
| | | yl]-4-hydroxy- |
| | | benzenesulfonamide |
| 059 | 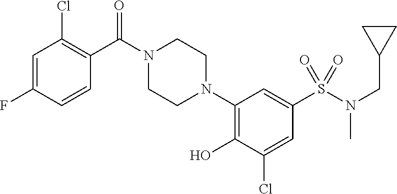 [SMILES: CN(CC1CC1)S(=O)(=O)c1cc(Cl)c(O)c(N2CCN(C(=O)c3ccc(F)cc3Cl)CC2)c1] | 3-chloro-5-[4-(2- chloro-4-fluoro- benzoyl)piperazin-1- yl]-N- (cyclopropylmethyl)-4- hydroxy-N-methyl- benzenesulfonamide |
| 060 | 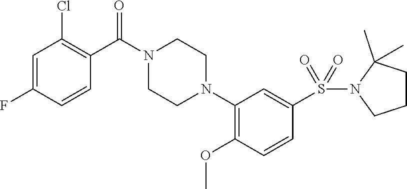 [SMILES: COc1ccc(S(=O)(=O)N2CCCC2(C)C)cc1N1CCN(C(=O)c2ccc(F)cc2Cl)CC1] | (2-chloro-4-fluoro- phenyl)-[4-[5-(2,2- dimethylpyrrolidin-1- yl)sulfonyl-2-methoxy- phenyl]piperazin-1- yl]methanone |

According to one preferred embodiment, the compound is selected from the compounds of Table 1 herein, and pharmaceutically acceptable salts and/or solvates thereof, provided that the compound is not N,N-diethyl-5-[4-[4-fluoro-2-(trifluoromethyl)benzoyl]piperazin-1-yl]-6-methoxy-pyridine-3-sulfonamide (045) or a pharmaceutically acceptable salt and/or solvate thereof.

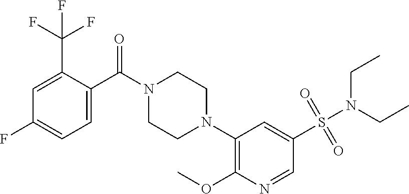 [SMILES: CCN(CC)S(=O)(=O)c1cnc(OC)c(N2CCN(C(=O)c3ccc(F)cc3C(F)(F)F)CC2)c1]

According to one embodiment, in the compound of formula (I), when R^{D} represents CF₃, R⁷ represents OCH₃ and Z represents C—H, then R^{A}, R^{B} and R^{C} do not represent F or Cl. According to one embodiment, in the compound of formula (I), when R^{D} represents CF₃ and R⁷ represents OCH₃, then R^{A}, R^{B} and R^{C} do not represent F. According to one embodiment, in the compound of formula (I), when R^{D} represents CF₃, then R^{A}, R^{B} and R^{C} do not represent F. In one embodiment, in the compound of formula (I), when R^{D} represents CF₃, then R^{A}, R^{B} and R^{C} do not represent F or Cl. According to one embodiment, in the compound of formula (I), when R⁷ represents OCH₃, then R^{A}, R^{B} and R^{C} do not represent F. In one embodiment, in the compound of formula (I), when R⁷ represents OCH₃, then R^{A}, R^{B} and R^{C} do not represent F or Cl. According to one embodiment, R^{D} does not represent CF₃. In one embodiment, R^{D} does not represent CF₃, CHF₂ or CH₂F. According to one embodiment, R⁷ does not represent methoxy (—OCH₃). In one embodiment, R⁷ does not represent alkoxy.

All references herein to a compound of the invention (e.g., “compound of formula (I)”) include references to salts, solvates, multi component complexes and liquid crystals thereof. All references herein to a compound of the invention include references to polymorphs and crystal habits thereof. All references herein to a compound of the invention include references to isotopically-labelled compounds thereof, including deuterated compounds thereof. All references herein to a compound of the invention include references to stereoisomers thereof. All references herein to a compound of the invention include references to pharmaceutically acceptable prodrugs thereof.

In particular, the compounds of the invention may be in the form of pharmaceutically acceptable salts. According to one embodiment, the compound of the invention is a pharmaceutically acceptable salt. Pharmaceutically acceptable salts include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts. Suitable base salts are formed from bases which form non-toxic salts. Examples include the aluminium, arginine, benzathine, calcium, choline, diethylamine, 2-(diethylamino)ethanol, diolamine, ethanolamine, glycine, 4-(2-hydroxyethyl)-morpholine, lysine, magnesium, meglumine, morpholine, olamine, potassium, sodium, tromethamine and zinc salts. Hemisalts of acids and bases may also be formed, for example, hemisulphate and hemicalcium salts. When a compound contains an acidic group as well as a basic group the compound may also form internal salts, and such compounds are within the scope of the invention. When a compound contains a hydrogen-donating heteroatom (e.g., NH), the invention also encompasses salts and/or isomers formed by transfer of said hydrogen atom to a basic group or atom within the molecule.

Pharmaceutically acceptable salts of compounds of the invention may be prepared by one or more of these methods: (i) by reacting the compound with the desired acid; (ii) by reacting the compound with the desired base; (iii) by removing an acid- or base-labile protecting group from a suitable precursor of the compound or by ring-opening a suitable cyclic precursor, e.g., a lactone or lactam, using the desired acid; and/or (iv) by converting one salt of the compound to another by reaction with an appropriate acid or by means of a suitable ion exchange column. All these reactions are typically carried out in solution. The salt may precipitate from solution and be collected by filtration or may be recovered by evaporation of the solvent. The degree of ionization in the salt may vary from completely ionized to almost non-ionized.

In particular, the compounds of the invention may be in the form of pharmaceutically acceptable solvates. According to one embodiment, the compound of the invention is a pharmaceutically acceptable solvate. According to one embodiment, the compound of the invention is a pharmaceutically acceptable salt and solvate.

In particular, the compounds of the invention may include at least one asymmetric center(s) and thus may exist as different stereoisomeric forms. Accordingly, all references herein to a compound of the invention include all possible stereoisomers and include not only the racemic compounds, but the individual enantiomers and their non-racemic mixtures as well. Non-racemic mixtures may comprise any amounts of each distinct stereoisomer, for example one stereoisomer may be preponderant (e.g., a 90/10 or 80/20 mixture), or the enantiomeric ratio may be close to a racemic mixture (e.g., a 40/60 mixture). When a compound is desired as a single enantiomer, such single enantiomer may be obtained by stereospecific synthesis, by resolution of the final product or any convenient intermediate, or by chiral chromatographic methods as each are known in the art. Resolution of the final product, an intermediate, or a starting material may be carried out by any suitable method known in the art.

### Pharmaceutical Composition

Another object of the present invention is a composition comprising a compound according to the invention, as described herein. According to one embodiment, the composition further comprises at least one pharmaceutically acceptable carrier, so that the composition is a “pharmaceutical composition” as defined herein.

In a first embodiment, the pharmaceutical composition comprises the compound according to the invention as sole therapeutic agent. In a second embodiment, the pharmaceutical composition further comprises at least another therapeutic agent such as, for example, a therapeutic agent suitable for treating a neurological disorder.

Another object of the present invention is a medicament comprising a compound according to the invention, as described herein.

### Kit

Another object of the present invention is a kit of parts (in short “kit”) comprising a compound or composition according to the invention, as described herein. According to one embodiment, the kit comprises a manufacture such as, for example, a package or a container. According to one embodiment, the kit comprises instructions for use. The kit may be promoted, distributed, or sold as a unit for performing the methods of the present invention.

According to one embodiment, the kit comprises: a pharmaceutical composition comprising the compound according to the invention, and another separate pharmaceutical composition comprising at least another therapeutic agent such as, for example, a therapeutic agent suitable for treating a neurological disorder.

### Medical Use of the Compound

Another object of the present invention is a compound or a composition according to the invention, as described herein, for use as a medicament.

Another object of the present invention is a compound or a composition according to the invention, as described herein, for use in the treatment of a neurological disorder.

Another object of the present invention is a method for treating a neurological disorder in a subject in need thereof. Another object of the present invention is the use of a compound or a composition according to the invention, as described herein, in the manufacture of a medicament for the treatment of a neurological disorder. Another object of the present invention is the use of a compound or a composition according to the invention, as described herein, for treating a neurological disorder.

According to one embodiment, the method or the use comprises a step of administering to a subject a therapeutically effective amount of a compound, a composition or a medicament according to the invention, as described herein.

According to one embodiment, the neurological disorder to be treated by the method or the use of the invention is:
a disease or a disorder associated with defective neurogenesis such as, for example, Hirschsprung disease, schizophrenia, Ataxia telangiectasia, age-related decline of nervous system performance, or a neurodevelopmental disorder,
a neurodegenerative disease or disorder, such as, for example, Alzheimer's disease, Parkinson's disease, Huntington's disease, Amyotrophic Lateral Sclerosis, Fronto-temporal dementia, retinal neurodegenerative diseases, neuro-ophthalmic diseases, neurotrophic keratitis, Charcot-Marie-Tooth disease, Spinal Muscular Atrophy, epilepsy (e.g., an epilepsy disorder, or a seizure disorder, or a chronic neurological disorder presenting with epilepsy), dementia, age-related decline of nervous system performance, a prion disease, Creutzfeldt-Jacob disease, Multiple System Atrophy (Shy Drager Syndrome), Multiple sclerosis, or Guillain-Barre syndrome;
a disease or a disorder associated with nerve injury or neurotoxicity, such as, for example, head injury, brain damage, traumatic brain injury, peripheral nerve injury, traumatic peripheral nerve injury, peripheral neuropathy, nerve transplantation complications, spinal cord injury, traumatic spinal cord injury, severance of nerves or nerve damage, severance of cerebrospinal nerve cord, a damage to brain or nerve cells, syringomyelia, optic neuropathy, trauma, stroke, ischemia, stroke, ischemic stroke, neurotoxicity caused by alcohol or substance abuse (e.g., ecstasy, methamphetamine etc.), or aphasia;
a neurodevelopmental disorder, such as, for example, Rett syndrome, X-linked mental retardation, fragile X syndrome, Down's syndrome, Autism Spectrum Disorder, Hirschsprung's disease, Tourette syndrome, childhood learning disorder, an attention deficit disorder, Attention Deficit Hyperactivity Disorder (ADHD), Angelman syndrome, micropreemie, schizophrenia, Language disorder, preterm birth, perinatal arterial ischemic stroke, spina bifida, mental retardation, nonsyndromic X-linked mental retardation, Ondine syndrome, or WAGR syndrome;
a neuropsychiatric disorder such as, for example, depression, Major Depressive Disorder, schizophrenia, schizophrenic form disorder, schizoaffective disorder, delusional disorder, anxiety, anxiety disorders, panic disorder, phobias, an obsessive-compulsive disorder, a post-traumatic stress disorder, a bipolar disorder, anorexia nervosa, bulimia nervosa, anhedonia, apathy, dementia, substance-induced dementia, a motor and/or tic disorder characterized by motor and/or vocal tics (e.g., Tourette's disorder), a substance use behavior, addiction, mood disorders, suicidality, cancer-related psychiatric symptoms, Alzheimer's disease, Huntington's disease, Fronto-temporal dementia, or a reward deficiency syndrome (RDS);
a movement disorder, such as, for example, Parkinson's disease, Huntington's disease, Amyotrophic Lateral Sclerosis, a motor and tic disorder characterized by motor and/or vocal tics (e.g., Tourette's disorder), an ataxia, ataxias muscular rigidity (spasticity), Charcot-Marie-Tooth disease, Spinal Muscular Atrophy, Werdnig-Hoffmann disease, or chronic proximal spinal muscular atrophy;
a pain disorder, such as, for example, neuralgia, trigeminal neuralgia, chronic pain, inflammatory pain, pain associated with arthritis, fibromyalgia, back pain, cancer-associated pain, pain associated with digestive disease, pain associated with Crohn's disease, pain associated with autoimmune disease, pain associated with endocrine disease, pain associated with diabetic neuropathy, phantom limb pain, spontaneous pain, chronic post-surgical pain, chronic, temporomandibular pain, causalgia, post-herpetic neuralgia, AIDS-related pain, complex regional pain syndromes type I and II, trigeminal neuralgia, chronic back pain, pain associated with spinal cord injury, pain associated with drug intake and recurrent acute pain, neuropathic pain, or inappropriate neuronal activity resulting in neurodystonia in a disease such as diabetes, an MS and a motor neuron disease;
an ophthalmic disease or an ocular disorder such as, for example, a retinal disorder, retinal neurodegenerative diseases, Retinitis Pigmentosa, Non-Arthritic Anterior Ischemic Optic Neuropathy (NAION), macular degeneration, age-related macular degeneration, glaucoma, diabetic retinopathy, optic neuropathy and retinal degeneration, neuro-ophthalmic diseases, age-related cataract, primary open-angle glaucoma (POAG), retinal ganglion cell damage, ocular hypertension, ischemic optic neuropathy, macular telangiectasia, cystoid macular edema, Macular Telangiectasia Type 2, neurotrophic keratitis, or strabismus;
a disorder of the enteric system or a gastro-intestinal disorder, such as, for example, a disorder of intestinal motility, constipation, Hirschsprung's disease, Inflammatory Bowel Disease, Intestinal Neuronal Dysplasia, ulcerative colitis, Achalasia, Esophageal spasm, duodenal ulcer, Zollinger-Ellison Syndrome, hypersecretion of gastric acid, malabsorptive disorder or intestinal inflammation;
a progressive muscular dystrophy, such as, for example, Duchenne, Becker, Emery-Dreifuss, Landowy-Dejerine, scapulohumeral, limb-girdle, Von Graefe-Fuchs, oculopharyngeal, myotonic and congenital, a congenital or acquired myopathy, Charcot-Marie-Tooth disease, Werdnig-Hoffmann disease, or chronic proximal spinal muscular atrophy;
a disease or a disorder associated with defective long-term or short-term memory, such as, for example, memory loss, benign forgetfulness, or Alzheimer's disease;
an autoimmune disorder such as, for example, multiple sclerosis, autoimmune encephalomyelitis, autoimmune encephalitis, autoimmune hemolytic anemia, chronic lymphocytic leukemia, Churg-Strauss syndrome, anti-N-methyl-D-aspartate receptor (NMDAR) encephalitis, Thyroid-associated orbitopathy, autoimmune thyroiditis, Guillain-Barré syndrome, or autoimmune thrombocytopenic purpura;
a neuro-ontological disease or disorder such as, for example, cochlear sensory cell damage, defective auditory perception, hearing loss, or tinnitus;
a sleep disorder, such as, for example, narcolepsy, restless leg syndrome, Obstructive sleep apnea, chronic insomnia disorder, paradoxical sleep deprivation or REM sleep deprivation;
a cerebrovascular disease or a neurovascular disease, such as, for example, early brain injury (EBI) after subarachnoid hemorrhage (SAH), cerebral ischemia, stroke, hypoxic-ischemic brain injury, perinatal arterial ischemic stroke, or neovascular Age-related macular degeneration (nvAMD);
a substance abuse disorder, such as, for example, substance dependence, substance abuse and the sequalae of substance abuse dependence, substance-induced psychological disorder, substance withdrawal and substance-induced dementia or amnestic disorder;
a neuronal reaction to viral infection, Trypanosoma infection, a neurological deficit associated with AIDS, obesity, temporomandibular joint dysfunction, aphasia, Bell's palsy, encephalitis, a kidney disease or renal dysfunction, phaeochromocytoma, a metabolic syndrome, cancer, eczema, thrombocytopenia; hypoplasia; disseminated intravascular coagulation (DIC); myelodysplasia; immune thrombocytopenic purpura (ITP), HIV induced ITP, a neuro-oncological disease or disorder, neuro-immunological disease or disorder, multiple endocrine neoplasia type 2, von Hippel-Lindau disease (VHL), type I neurofibromatosis, Scleroderma, an epidermal and stromal wound healing disorder and/or a scarring disorder; or
a disease or disorder associated with aging and/or senescence.

According to one embodiment, the neurological disorder is epilepsy, such as, for example, Dravet syndrome, benign Rolandic epilepsy, frontal lobe epilepsy, infantile spasms, juvenile myoclonic epilepsy (JME), juvenile absence epilepsy, childhood absence epilepsy (e.g., pyknolepsy), febrile seizures, progressive myoclonus epilepsy of Lafora, Lennox-Gastaut syndrome, Landau-Kleffner syndrome, Generalized Epilepsy with Febrile Seizures (GEFS+), Severe Myoclonic, Epilepsy of Infancy (SMEI), Benign Neonatal Familial Convulsions (BFNC), West Syndrome, Ohtahara Syndrome, early myoclonic encephalopathies, migrating partial epilepsy, infantile epileptic encephalopathies, Tuberous Sclerosis Complex (TSC), focal cortical dysplasia, Type I Lissencephaly, Miller-Dieker Syndrome, Angelman's syndrome, Fragile X syndrome, epilepsy in autism spectrum disorders, epilepsy in subcortical band heterotopia, epilepsy in Walker-Warburg syndrome, epilepsy in Alzheimer's disease, post-traumatic epilepsy, progressive myoclonus epilepsies, reflex epilepsy, Rasmussen's syndrome, temporal lobe epilepsy, limbic epilepsy, status epilepticus, abdominal epilepsy, massive bilateral myoclonus, catamenial epilepsy, Jacksonian seizure disorder, Unverricht-Lundborg disease, or photosensitive epilepsy.

According to one embodiment, the composition or the medicament according to the invention, as described herein, is to be administered to a subject, and may be formulated using methods well-known in the art. Non-limiting examples of forms adapted for administration include solutions (such as, for example, sterile aqueous solutions), gels, dispersions, emulsions, suspensions and solid forms suitable for using to prepare solutions or suspensions upon the addition of a liquid prior to use (such as, for example, powder or liposomal forms).

The composition or the medicament according to the invention, as described herein, may be administered using administration route well-known in the art such as, for example, parenterally, orally, by inhalation, spray, rectally, nasally, or via an implanted reservoir.

It will be however understood that the total daily usage of the compound, the composition or the medicament will be decided by the attending physician within the scope of sound medical judgment. The specific therapeutically effective dose level for any particular patient will depend upon a variety of factors including the disease being treated and the severity of the disease; activity of the compound employed; the age, body weight, general health, sex and diet of the subject; the time of administration, route of administration, and rate of excretion of the specific therapeutic agent employed; the duration of the treatment; drugs used in combination or coincidental with the specific therapeutic agent employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

In one embodiment, the dosage of the compound is about 0.01 to 500 mg per kg patient body weight per day, which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be about 0.05 to 0.5, about 0.5 to 5 or about 5 to 50 mg/kg per day. For oral administration, the compositions are preferably provided in the form of tablets containing from about 1.0 to 1000 milligrams of the active ingredient, particularly about 1.0, about 5.0, about 10.0, about 15.0, about 20.0, about 25.0, about 50.0, about 75.0, about 100.0, about 150.0, about 200.0, about 250.0, about 300.0, about 400.0, about 500.0, about 600.0, about 750.0, about 800.0, about 900.0, and about 1000.0 milligrams of the active ingredient for the symptomatic adjustment of the dosage to the patient to be treated. The compounds may be administered on a regimen of 1 to 4 times per day, preferably once or twice per day. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

According to a first embodiment, the composition or the medicament according to the invention, as described herein, is to be administered as sole therapeutic agent. According to a second embodiment, the composition or the medicament according to the invention, as described herein, is to be administered before at least another therapeutic agent, concomitantly with at least another therapeutic agent, and/or after at least another therapeutic agent. In one embodiment, the other therapeutic agent is suitable for treating a neurological disorder.

Another object of the present invention is a method of promoting neuronal cell's survival and/or neuronal cell's functions. Another object of the present invention is a compound or a composition according to the invention, as described herein, for use in promoting neuronal cell's survival and/or neuronal cell's functions. According to one embodiment, the method or the use comprises a step of contacting a neuronal cell with a therapeutically effective amount of a compound, a composition or a medicament according to the invention, as described herein. The method or the use may be in vitro, ex vivo or in vivo.

Another object of the present invention is a method of rescuing neuronal cell's functions after the neuronal cell has been subjected to insults, events, or conditions detrimental to neuronal cell's functions. Another object of the present invention is a compound or a composition according to the invention, as described herein, for use in rescuing neuronal cell's functions after the neuronal cell has been subjected to insults, events, or conditions detrimental to neuronal cell's functions. Such insults, events, or conditions include, without limitation, neuronal stress, for instance, caused by hypoxia or ischemia; traumatic injuries; and exposure to toxic molecules, for instance, to abnormal misfolded proteins, protein aggregates, excitotoxins, reactive oxygen species, endoplasmic reticulum stressors, mitochondrial stressors, Golgi apparatus antagonists and the like. According to one embodiment, the method or the use comprises a step of contacting a neuronal cell with a therapeutically effective amount of a compound, a composition or a medicament according to the invention, as described herein. The method or the use may be in vitro, ex vivo or in vivo.

Another object of the present invention is a method of binding or modulating GFRα1 using a compound or a composition according to the invention, as described herein. Another object of the present invention is a compound or a composition according to the invention, as described herein, for binding or modulating GFRα1. According to one preferred embodiment, the compound or a composition is for activating GFRα1. In one embodiment, GFRα1 is human GFRα1, preferably with SEQ ID NO: 1.

Another object of the present invention is a method of activating the GFRα1/RET signaling pathway using a compound or a composition according to the invention, as described herein. Another object of the present invention is a compound or a composition according to the invention, as described herein, for activating the GFRα1/RET signaling pathway.

Another object of the present invention is a method of detecting GFRα1 in a sample, using a compound or a composition according to the invention, as described herein. Another object of the present invention is a compound or a composition according to the invention, as described herein, for detecting GFRα1 in a sample. In one embodiment, GFRα1 is human GFRα1, preferably with SEQ ID NO: 1. In one embodiment, the compound according to the invention may be fused to a detectable label, such as, e.g., a fluorophore or any other moiety that can re-emit light upon light excitation, a radiolabel, a contrast agent and the like.

### Manufacturing Process

### Synthesis of the Compound

The compound according to the invention, as described herein, may be manufactured by means of synthetic methods well-known in the art.

Another object of the present invention is a process for manufacturing a compound of the invention, as described herein. According to one embodiment, the process is a Buchwald-Hartwig amination.

According to one embodiment, the process comprises:
(a-1) a step of reaction of
a compound of formula (II)

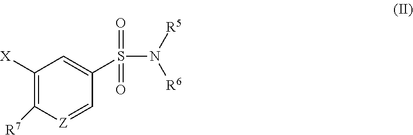

wherein Z, R⁵, R⁶ and R⁷ are as defined under formula (I) herein and X represents halide or —CF₃SO₃,
with a compound of formula (III)

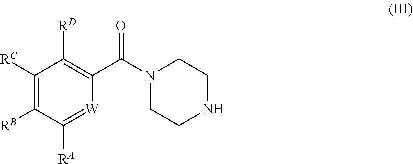

wherein W and R^{A}-R^{D} are as defined under formula (I) herein,
in presence of a base and a metal catalyst; thereby obtaining the compound of the invention.

In one embodiment, the base at step (a-1) is cesium carbonate (Cs₂CO₃), sodium carbonate (Na₂CO₃) or potassium carbonate (K₂CO₃). In one particular embodiment, the base is cesium carbonate (Cs₂CO₃). In one embodiment, the base is sodium tert-butanoate (t-BuONa), potassium tert-butanoate (t-BuOK) or potassium phosphate. In one particular embodiment, the base is sodium tert-butanoate (t-BuONa).

In one embodiment, the catalyst at step (a-1) is a palladium catalyst. In one particular embodiment, the catalyst is a Pd(OAc)₂ and rac-BINAP (2,2′-bis(diphenylphosphino)-1,1′-binaphtyle) system. In one particular embodiment, the catalyst is XPhos-Pd-G3.

In one embodiment, X represents halide. In one particular embodiment, X represents Br.

In one embodiment, the reaction at step (a-1) is carried out in a solvent. In one particular embodiment, the solvent is toluene. In one embodiment, the reaction is carried out at reflux.

According to another embodiment, the process comprises:
(a′-1) a step of reaction of
a compound of formula (II)

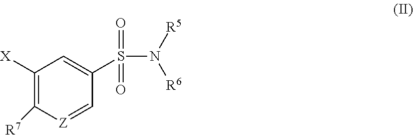

wherein Z, R⁵, R⁶ and R⁷ are as defined under formula (I) herein and X represents halide or —CF₃SO₃,
with a mono-protected piperazine (i.e., a piperazine wherein only one of the NH groups is protected by means of a protecting group R^{P}),
in presence of a base and a metal catalyst;
thereby obtaining a compound of formula (IV)

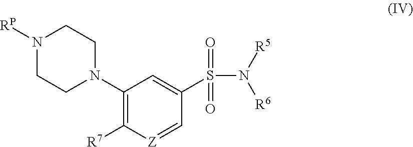

wherein Z, R⁵, R⁶ and R⁷ are as defined under formula (II) above and R^{P} is the protecting group; then
(a′-2) a step of deprotection of the compound of formula (IV);
thereby obtaining a compound of formula (V)

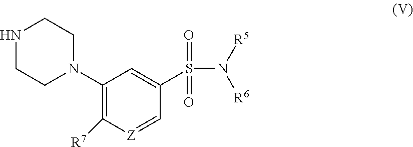

wherein Z, R⁵, R⁶ and R⁷ are as defined under formula (II) above; and
(a′-3) a step of reaction of the compound of formula (V) with a compound of formula (VI)

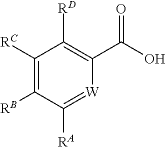

wherein W and R^{A}-R^{D} are as defined under formula (I) herein, in presence of a peptide coupling agent and a base;
thereby obtaining the compound of the invention.

In one embodiment, the base and/or the catalyst at step (a′-1) are as described hereinabove under step (a-1). In one embodiment, X represents halide. In one particular embodiment, X represents Br.

The protecting group may be any protecting group known in the art such as, for example, tert-butyloxycarbonyl (Boc). The protective group may be removed at step (a′-2) by any method known in the art appropriate to the nature of the protective group, such as, for example, addition of a strong Bronsted acid (e.g., hydrochloric acid [HCl]).

The peptide coupling agent at step (a′-3) may be any peptide coupling agent known in the art such as, for example, 2-(1H-benzotriazole-1-yl)-1,1,3,3-tetramethylaminium tetrafluoroborate (TBTU).

The base at step (a′-3) may be any base known in the art such as, for example, an amine base. According to one embodiment, the amine is triethylamine (Et₃N) or diisopropylethylamine (iPr₂NEt).

In one embodiment, the reaction at step (a′-3) is carried out in a solvent. In one particular embodiment, the solvent is dimethylformamide (DMF) and/or tetrahydrofuran (THF). In one particular embodiment, the solvent is dichloroethane (DCE) and/or acetonitrile (MeCN). In one embodiment, the reaction is carried out at room temperature (RT).

According to another embodiment, the process comprises:
(a″-1) a step of reaction of:
a compound of formula (III)

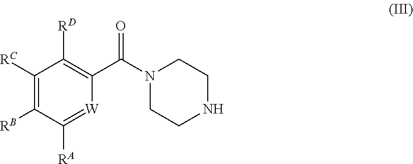

wherein W and R^{A}-R^{D} are as defined under formula (I) herein,
with a compound of formula (VII)

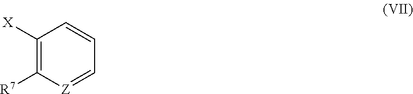

wherein R⁷ and Z are as defined under formula (I) herein, except that Z does not represent N, and X represents halide or —CF₃SO₃,
in presence of a base and a metal catalyst;
thereby obtaining a compound of formula (VIII)

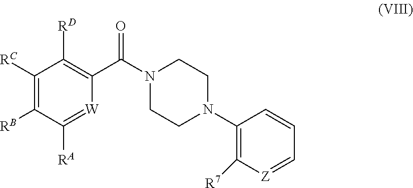

wherein W, R^{A}-R^{D}, R⁷ and Z are as defined under formulae (III) and (VII) above;
(a″-2) a step of reaction of the compound of formula (VIII) with chlorosulfonic acid (HSO₃Cl);
thereby obtaining a compound of formula (IX);

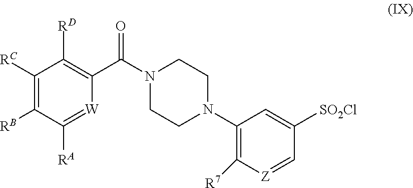

wherein W, R^{A}-R^{D}, R⁷ and Z are as defined under formulae (III) and (VII) above;
(a″-3) a step of reaction of the compound of formula (IX) with a secondary amine of formula (X)

NHR⁵R⁶ (X)
wherein R⁵ and R⁶ are as defined under formula (I) herein,
in presence of a base;
thereby obtaining the compound of the invention.

According to an alternative embodiment, the process comprises a step (a″-1) of reaction of a compound of formula (III) with a compound of formula (VII) as described hereinabove, thereby obtaining a compound of formula (VIII); but no step (a″-2) of reaction of the compound of formula (VIII) with chlorosulfonic acid (HSO₃Cl); and Z may represent N.

In one embodiment, the base and/or the catalyst at step (a″-1) are as described hereinabove under step (a-1). In one embodiment, X represents halide. In one particular embodiment, X represents Br.

The base at step (a″-3) may be any base known in the art such as, for example, an amine base. According to one embodiment, the amine is triethylamine (Et₃N) or diisopropylethylamine (iPr₂NEt).

In one embodiment, the reaction at step (a″-3) is carried out in a solvent. In one particular embodiment, the solvent is dichloromethane (DCM).

In one embodiment, the process further comprises a work-up step (b). In one embodiment, the work-up step (b) comprises a step of extraction by a solvent. In one particular embodiment, the solvent is ethyl acetate (EtOAc). In one particular embodiment, the solvent is water or a 1N HCl solution. In one particular embodiment, the solvent is dichloromethane (DCM). In one embodiment, the work-up step (b) comprises a step of filtration. In one particular embodiment, the filtration is over Celite® In one embodiment, the work-up step (b) comprises a step of concentration under reduced pressure.

In one embodiment, the process further comprises a purification step (c). In one embodiment, the purification step (c) comprises a purification by chromatography. In one particular embodiment, the chromatography is flash chromatography (FC) (e.g., cHex/EtOAc gradient), preparative thin-layer chromatography (PTLC) or semi-preparative high-performance liquid chromatography (HPLC).

### Synthetic Intermediates

Another object of the present invention is a compound of formula (II)

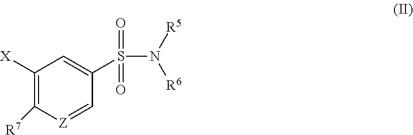

wherein Z, R⁵, R⁶ and R⁷ are as defined under formula (I) herein and X represents halide or —CF₃SO₃.

Another object of the present invention is a compound of formula (III)

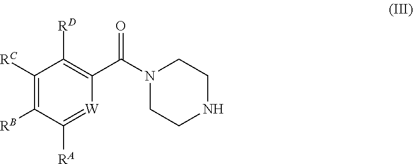

wherein W and R^{A}-R^{D} are as defined under formula (I) herein.

Another object of the present invention is a compound of formula (IV)

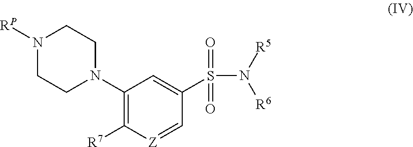

wherein Z, R⁵, R⁶ and R⁷ are as defined under formula (I) herein and R^{P} is a protecting group (e.g., Boc).

Another object of the present invention is a compound of formula (V)

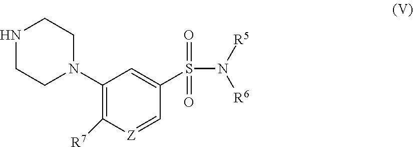

wherein Z, R⁵, R⁶ and R⁷ are as defined under formula (I) herein.

Another object of the present invention is a compound of formula (VIII)

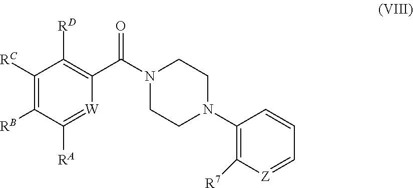

wherein W, R^{A}-R^{D}, R⁷ and Z are as defined under formula (I) herein.

According to one preferred embodiment, in formula (VIII) hereinabove, Z does not represent N.

Another object of the present invention is a compound of formula (IX)

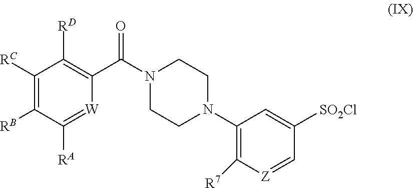

wherein W, R^{A}-R^{D}, R⁷ and Z are as defined under formula (I) herein.

According to one preferred embodiment, in formula (IX) hereinabove, Z does not represent N.

### Examples

The present invention is further illustrated by the following examples.

### Example 1: Synthesis of the Compounds

### General Material and Methods

### ABBREVIATIONS

### List of Abbreviations

Ac: acetyl
Ar: argon
BINAP: (2,2′-bis(diphenylphosphino)-1,1′-binaphthyl)
t-Bu: tert-butyl
cHex: cyclohexane
dba: dibenzylideneacetone
DCM: dichloromethane
DCE: dichloroethane
DMF: dimethylformamide
Et: ethyl
FC: flash chromatography
HPLC: high-performance liquid chromatography
iPr: isopropyl
PTLC: preparative thin-layer chromatography
RT: room temperature
TBTU: 2-(1H-benzotriazole-1-yl)-1,1,3,3-tetramethylaminium tetrafluoroborate
THF: tetrahydrofuran
TPTU: 0-(2-Oxo-1(2H)pyridyl)-N,N,N′,N′-tetramethyluronium tetrafluoroborate
XPhos: dicyclohexyl[2′,4′,6′-tris(propan-2-yl)[1,1′-biphenyl]-2-yl]phosphane
XantPhos: (9,9-dimethyl-9H-xanthene-4,5-diyl)bis(diphenylphosphane)

### Analytical Methods

¹H NMR spectra (400 MHz) and ¹⁹F NMR spectra (376 MHz) were recorded with a Bruker ULTRASHIELD 400 spectrometer. Processing and analyses of the spectra were performed with MestReNova. Data appear in the following order: chemical shifts in ppm which were referenced to the internal solvent signal, multiplicity, coupling constant J in Hertz and number of protons.

Reversed-phase HPLC/MS analyses were carried out with a Waters Alliance 2795 HPLC equipped with an autosampler, an inline membrane degasser, a column oven 10 (T°=45 °C), a UV detector, and a ZQ quadrupole mass detector working in ionization electrospray mode. Analyzed compounds (0.1 to 0.3 mg) were solubilized in a minimum amount of DMSO completed with acetonitrile (total volume: 1 mL). Standard analytical parameters: flow rate: 1 mL/min, Vᵢₙⱼ: 5 μL. Acidic conditions: Waters XSelect CSH C18 column (3.5 μm, 2.1×15 50 mm). Gradient: (H₂O+0.04% v/v HCO₂H (10 mM))/ACN from 95/5 to 0/100 in 2.5 min. Alkaline conditions: Waters Xbridge C18 column (3.5 μm, 2.1×50 mm). Gradient: (H₂O+0.06% v/v NH₃ (aq.) (10 mM))/ACN from 95/5 to 0/100 in 2.5 min.

### General Synthetic Methods

### General Protocol 1 (GP-1): Peptide Coupling Using TBTU

To a solution of the required carboxylic acid in DMF at RT, was added TBTU. The mixture was stirred for 15 min at RT and a solution of the required piperazine and Et₃N in THF was added dropwise. The mixture was stirred at RT for the required time. The volatiles were removed under reduced pressure and the residue was partitioned between 1N HCl and EtOAc. The layers were separated and the aqueous phase was extracted with EtOAc. The combined organic extracts were washed (1N HCl, aq. sat. NaHCO₃, brine), dried (Na₂SO₄), filtered and concentrated under reduced pressure. The residue was purified by FC (cHex/EtOAc gradient) or PTLC to afford the required product.

### General Protocol 2 (GP-2): Peptide Coupling Using TPTU (Small Scale)

To a solution of the required carboxylic acid in DCE/MeCN (1/1) at RT, were added iPr₂NEt and TPTU. After stirring for 5 min at RT, the required piperazine was added and the mixture was stirred at RT for the required time. 2 drops of ethylene diamine were added and the reaction mixture was concentrated under reduced pressure. The residue was purified by FC (cHex/EtOAc gradient), PTLC or semi-preparative HPLC to afford the required product.

### General Protocol 3 (GP-3): Chlorosulfonylation

To chlorosulfonic acid at 0 °C, was added dropwise a solution of the required arene in DCM. The mixture was allowed to warm-up to RT and allowed to stir at RT for the required time. The reaction mixture was poured dropwise on crushed ice while stirring (very exothermic quench). At the end of the addition, the residual ice was allowed to melt, DCM was added and the layers were separated. The aqueous phase was extracted with DCM and the combined organic extracts were dried (Na₂SO₄), filtered and concentrated under reduced pressure to afford the desired sulfonyl chloride.

### General Protocol 4 (GP-4): Sulfonamide Formation

To a solution of the required sulfonyl chloride in DCM at RT, was added a solution of the required amine and either Et₃N or iPr₂NEt in DCM. The reaction mixture was stirred at RT for the required time. The reaction mixture was partitioned between EtOAc and aq. sat. NH₄Cl. The layers were separated and the aqueous phase was extracted with EtOAc. The combined organic extracts were washed (aq. sat. NH₄Cl, brine), dried (Na₂SO₄), filtered and concentrated under reduced pressure. The residue was purified by FC (cHex/EtOAc gradient), PTLC or semi-preparative HPLC to afford the required product.

### General Protocol 5 (GP-5): Buchwald Coupling Using Pd(OAc)₂/Rac-BINAP

A microwave reaction vial was charged with the required arylbromide, the required piperazine, Cs₂CO₃, Pd(OAc)₂ and rac-BINAP. The vial was flushed with argon and degassed toluene was added. The vial was sealed and the reaction was stirred at reflux in a preheated heating-block for the required time. After cooling down to RT, EtOAc was added and the suspension as filtered over Celite® (EtOAc rinses). The filtrate was concentrated under reduced pressure. The residue was purified by FC (cHex/EtOAc gradient) or PTLC to afford the required product.

### General Protocol 6 (GP-6): Buchwald Coupling Using XantPhos-Pd-G3

A microwave reaction vial was charged with the required arylbromide, the required piperazine, t-BuONa and XantPhos-Pd-G3. The vial was flushed with argon and degassed toluene was added. The vial was sealed and the reaction was stirred at reflux in a preheated heating-block for the required time. After cooling down to RT, EtOAc was added and the suspension as filtered over Celite® (EtOAc rinses). The filtrate was concentrated under reduced pressure. The residue was purified by FC (cHex/EtOAc gradient) or PTLC to afford the required product.

### General Protocol 7 (GP-7): Buchwald Coupling Using XPhos-Pd-G3

A microwave reaction vial was charged with the required arylbromide, the required piperazine, t-BuONa and XPhos-Pd-G3. The vial was flushed with argon and degassed toluene was added. The vial was sealed and the reaction was stirred at reflux in a preheated heating-block for the required time. After cooling down to RT, EtOAc was added and the suspension as filtered over Celite® (EtOAc rinses). The filtrate was concentrated under reduced pressure. The residue was purified by FC (cHex/EtOAc gradient) or PTLC to afford the required product.

### Synthesis of Intermediate Compounds

Intermediate compound [4-fluoro-2-(trifluoromethyl)phenyl]-piperazin-1-yl-methanone was prepared as described by JI, D. et al.: “Design, synthesis and biological evaluation of anthranilamide derivatives as potent SMO inhibitors”, Bioorganic and medicinal chemistry, 5 Feb. 2020, Vol. 28, No. 6, pp. 1-12.

Intermediate compound 3-bromo-N,N-diethyl-benzenesulfonamide was prepared as described by SUTHERLAND, M. et al.: “Rational Design and Synthesis of Selective PRMT4 Inhibitors: A New Chemotype for Development of Cancer Therapeutics”, ChemMedChem, 29 Jan. 2021, Vol. 16, No. 7, pp. 1116-1125.

### Synthesis of Intermediates Required for Final Products 001 and 002

### 3-bromo-NN-diethyl-4-methoxy-benzenesulfonamide (I-001)

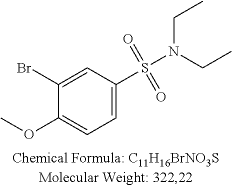 [SMILES: CCN(CC)S(=O)(=O)c1ccc(OC)c(Br)c1]

According to GP-4, I-001 was obtained as a yellow oil in 93% yield using 3-bromo-4-methoxy-benzenesulfonyl chloride (5.00 g, 17.5 mmol, 1 equiv.), diethylamine (2.72 mL, 26.3 mmol, 1.5 equiv.) and triethylamine (3.66 mL, 26.3 mmol, 1.5 equiv.) in DCM (70 mL) for 16 h at RT. ¹H NMR (400 MHz, Chloroform-d) δ 7.91 (d, J=2.4 Hz, 1H), 7.67 (dd, J=8.8, 2.4 Hz, 1H), 6.88 (d, J=8.8 Hz, 1H), 3.89 (s, 3H), 3.15 (q, J=7.2 Hz, 4H), 1.07 (t, J=7.2 Hz, 6H).

### tert-butyl 4-[5-(diethylsulfamoyl)-2-methoxy-phenyl]piperazine-1-carboxylate (I-002)

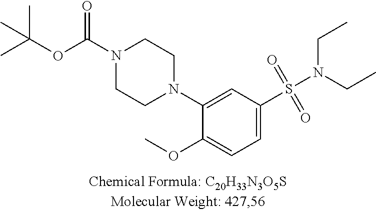 [SMILES: CCN(CC)S(=O)(=O)c1ccc(OC)c(N2CCN(C(=O)OC(C)(C)C)CC2)c1]

According to GP-5, I-002 was obtained as a dark oil in 93% yield using I-001 (5.20 g, 16.1 mmol, 1 equiv.), tert-butyl piperazine-1-carboxylate (4.51 g, 24.2 mmol, 1.5 equiv.), Cs₂CO₃ (15.8 g, 48.4 mmol, 3 equiv.), Pd(OAc)₂ (363 mg, 1.61 mmol, 0.1 equiv.) and rac-BINAP (1.51 g, 2.42 mmol, 0.15 equiv.) in toluene (81 mL) at reflux for 16 h. Purification by FC (cHex/EtOAc=95/5 to 40/60). ¹H NMR (400 MHz, DMSO-d₆) δ 7.42 (dd, J=8.5, 2.2 Hz, 1H), 7.17-7.08 (m, 2H), 3.88 (s, 3H), 3.47 (t, J=5.0 Hz, 4H), 3.13 (q, J=7.1 Hz, 4H), 2.95 (t, J=5.0 Hz, 4H), 1.43 (s, 9H), 1.03 (t, J=7.1 Hz, 6H).

### N,N-diethyl-4-methoxy-3-piperazin-1-yl-benzenesulfonamide (I-003)

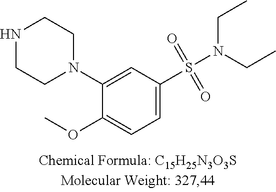 [SMILES: CCN(CC)S(=O)(=O)c1ccc(OC)c(N2CCNCC2)c1]

To a solution of I-002 (6.48 g, 15.2 mmol, 1 equiv.) in dioxane (38 mL) at 0 °C, was added HCl (4M solution in dioxane, 38.0 mL, 152 mmol, 10 equiv.). The mixture was stirred at RT for 16 h. The reaction mixture was concentrated under reduced pressure and the residue was partitioned between water and DCM. Na₂CO₃(s) was added portionwise until pH>11. The layers were separated and the aqueous phase was extracted with DCM (2*). The combined organic extracts were dried (Na₂SO₄), filtered and concentrated under reduced pressure. The residue was purified by FC (DCM/MeOH(7N NH₃)=99:1 to 90:10) to afford 4.87 g (98%) of I-003 as a colorless oil. ¹H NMR (400 MHz, DMSO-d₆) δ 7.39 (dd, J=8.5, 2.3 Hz, 1H), 7.20-7.05 (m, 2H), 3.87 (s, 3H), 3.43 (m, 1H), 3.13 (q, J=7.1 Hz, 4H), 3.05-2.56 (m, 8H), 1.03 (t, J=7.1 Hz, 6H). MS (ESI⁺): [M+H]⁺ 328.

### Synthesis of Intermediates Required for Final Products 003 and 004

### 1-(3-bromo-4-methoxy-phenyl)sulfonylpyrrolidine (I-004)

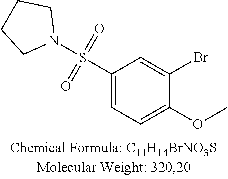 [SMILES: COc1ccc(S(=O)(=O)N2CCCC2)cc1Br]

According to GP-4, I-004 was obtained as a white solid in 95% yield using 3-bromo-4-methoxy-benzenesulfonyl chloride (5.00 g, 17.5 mmol, 1 equiv.), pyrrolidine (2.16 mL, 26.3 mmol, 1.5 equiv.) and triethylamine (3.66 mL, 26.3 mmol, 1.5 equiv.) in DCM (70 mL) for 1 h at RT. ¹H NMR (400 MHz, DMSO-d₆) δ 7.91 (d, J=2.3 Hz, 1H), 7.81 (dd, J=8.7, 2.2 Hz, 1H), 7.32 (d, J=8.7 Hz, 1H), 3.95 (s, 3H), 3.13 (m, 4H), 1.66 (m, 4H). MS (ESI⁺): [M+H]⁺ 320/322.

### tert-butyl 4-(2-methoxy-S-pyrrolidin-1-ylsulfonyl-phenyl)piperazine-1-carboxylate (I-005)

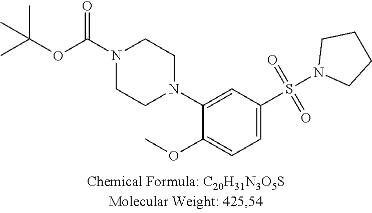 [SMILES: COc1ccc(S(=O)(=O)N2CCCC2)cc1N1CCN(C(=O)OC(C)(C)C)CC1]

According to GP-5, I-005 was obtained as a dark oil in 83% yield using I-004 (5.10 g, 15.9 mmol, 1 equiv.), tert-butyl piperazine-1-carboxylate (4.45 g, 23.9 mmol, 1.5 equiv.), Cs₂CO₃ (15.6 g, 47.8 mmol, 3 equiv.), Pd(OAc)₂ (358 mg, 1.59 mmol, 0.1 equiv.) and rac-BINAP (1.49 g, 2.39 mmol, 0.15 equiv.) in toluene (80 mL) at reflux for 28 h. Purification by FC (cHex/EtOAc=95/5 to 40/60). ¹H NMR (400 MHz, DMSO-d₆) δ 7.44 (dd, J=8.5, 2.2 Hz, 1H), 7.17 (s, 1H), 7.16 (d, J=5.2 Hz, 1H), 3.89 (s, 3H), 3.47 (t, J=4.9 Hz, 4H), 3.15-3.07 (m, 4H), 2.96 (t, J=5.0 Hz, 4H), 1.71-1.59 (m, 4H), 1.43 (s, 9H). MS (ESI⁺): [M+H]+ 426.

### I-(2-methoxy-5-pyrrolidin-1-ylsulfonyl-phenyl)piperazine (I-006)

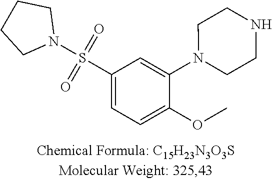 [SMILES: COc1ccc(S(=O)(=O)N2CCCC2)cc1N1CCNCC1]

To a solution of I-005 (5.90 g, 13.9 mmol, 1 equiv.) in dioxane (35 mL) at 0 °C, was added HCl (4M solution in dioxane, 39.0 mL, 139 mmol, 10 equiv.). The mixture was stirred at RT for 16 h. The reaction mixture was concentrated under reduced pressure and the residue was partitioned between water and DCM. Na₂CO₃(s) was added portionwise until pH>11. The layers were separated and the aqueous phase was extracted with DCM (2*). The combined organic extracts were dried (Na₂SO₄), filtered and concentrated under reduced pressure. The residue was purified by FC (DCM/MeOH(7N NH₃)=99:1 to 90:10) to afford 4.37 g (97%) of I-006 as a colorless oil. ¹H NMR (400 MHz, DMSO-d₆) δ 7.41 (dd, J=8.5, 2.2 Hz, 1H), 7.16 (m, 2H), 4.11 (m, 1H), 3.88 (s, 3H), 3.16-3.06 (m, 4H), 3.05-2.99 (m, 4H), 2.91 (m, 4H), 1.63-1.66 (m, 4H). MS (ESI⁺): [M+H]+ 326.

### Synthesis of Intermediates Required for Final Products 005 and 006

### 3-bromo-4-methoxy-N-methyl-N-propyl-benzenesulfonamide (I-007)

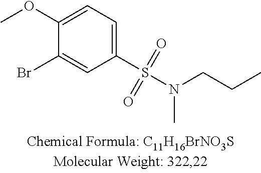 [SMILES: CCCN(C)S(=O)(=O)c1ccc(OC)c(Br)c1]

According to GP-4, I-007 was obtained as a yellow oil in 96% yield using 3-bromo-4-methoxy-benzenesulfonyl chloride (5.00 g, 17.5 mmol, 1 equiv.), N-methylpropylamine (2.69 mL, 26.3 mmol, 1.5 equiv.) and triethylamine (3.66 mL, 26.3 mmol, 1.5 equiv.) in DCM (70 mL) for 16 h at RT. ¹H NMR (400 MHz, Chloroform-d) δ 7.98 (d, J=2.2 Hz, 1H), 7.74 (dd, J=8.6, 2.2 Hz, 1H), 6.99 (d, J=8.6 Hz, 1H), 3.99 (s, 3H), 3.02-2.96 (m, 2H), 2.74 (s, 3H), 1.65-1.52 (m, 2H), 0.95 (t, J=7.4 Hz, 3H). MS (ESI⁺): [M+H]⁺ 322/324.

### tert-butyl 4-[2-methoxy-5-[methyl(propyl)sulfamoyl]phenyl]piperazine-1-carboxylate (I-008)

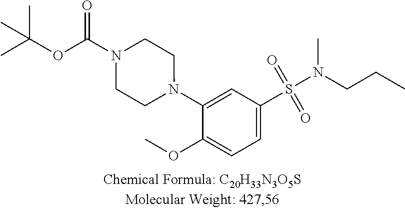 [SMILES: CCCN(C)S(=O)(=O)c1ccc(OC)c(N2CCN(C(=O)OC(C)(C)C)CC2)c1]

According to GP-5, I-005 was obtained as a dark oil in 74% yield using I-007 (1.71 g, 5.50 mmol, 1 equiv.), tert-butyl piperazine-1-carboxylate (1.54 g, 8.24 mmol, 1.5 equiv.), Cs₂CO₃ (5.38 g, 16.5 mmol, 3 equiv.), Pd(OAc)₂ (123 mg, 0.550 mmol, 0.1 equiv.) and rac-BINAP (513 mg, 0.824 mmol, 0.15 equiv) in toluene (80 mL) at reflux for 28 h. Purification by FC (cHex/EtOAc=98/2 to 50/50). ¹H NMR (400 MHz, Chloroform-d) δ 7.46 (dd, J=8.5, 2.2 Hz, 1H), 7.26 (d, J=2.6 Hz, 1H), 6.93 (d, J=8.6 Hz, 1H), 3.94 (s, 3H), 3.64-3.53 (m, 4H), 3.02 (t, J=5.0 Hz, 4H), 2.94 (dd, J=7.9, 6.6 Hz, 2H), 2.69 (s, 3H), 1.55-1.50 (m, 2H), 1.49 (s, 9H), 0.93 (d, J=7.3 Hz, 3H). MS (ESI⁺): [M+H]⁺ 428.

### 4-methoxy-N-methyl-3-piperazin-1-yl-N-propyl-benzenesulfonamide (I-009)

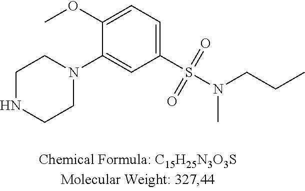 [SMILES: CCCN(C)S(=O)(=O)c1ccc(OC)c(N2CCNCC2)c1]

To a solution of I-008 (3.70 g, 8.65 mmol, 1 equiv.) in DCM (47 mL) at 0 °C, was added HCl (4M solution in dioxane, 21.6 mL, 86.5 mmol, 10 equiv.). The mixture was stirred at RT for 16 h. The reaction mixture was concentrated under reduced pressure and the residue was partitioned between water and DCM. Na₂CO₃(s) was added portionwise until pH>11. The layers were separated and the aqueous phase was extracted with DCM (2*). The combined organic extracts were dried (Na₂SO₄), filtered and concentrated under reduced pressure. The residue was purified by FC (DCM/MeOH(7N NH₃)=99:1 to 90:10) to afford 2.70 g (96%) of I-009 as an orange oil. ¹H NMR (400 MHz, Chloroform-d) δ 7.44 (dd, J=8.5, 2.2 Hz, 1H), 7.28 (d, J=2.3 Hz, 1H), 6.93 (d, J=8.6 Hz, 1H), 3.93 (s, 3H), 3.10-3.00 (m, 8H), 2.94 (t, J=7.2 Hz, 2H), 2.70 (s, 3H), 1.93 (s, 1H), 1.54 (h, J=7.4 Hz, 2H), 0.92 (t, J=7.4 Hz, 3H).MS (ESI⁺): [M+H]⁺ 328.

### Synthesis of Intermediates Required for Final Products 007 to 010

### 4-benzyl-1-(3-bromo-4-methoxy-phenyl)sulfonyl-piperidine (I-010)

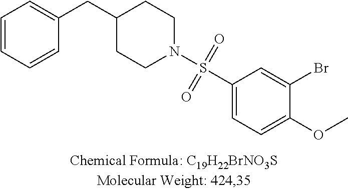 [SMILES: COc1ccc(S(=O)(=O)N2CCC(Cc3ccccc3)CC2)cc1Br]

According to GP-4, I-010 was obtained as a yellow oil in 100% yield using 3-bromo-4-methoxy-benzenesulfonyl chloride (1.00 g, 3.50 mmol, 1 equiv.), 4-benzylpiperidine (798 mg, 4.55 mmol, 1.3 equiv.) and triethylamine (0.73 mL, 5.2 mmol, 1.5 equiv.) in DCM (14 mL) for 16 h at RT. ¹H NMR (400 MHz, Chloroform-d) δ 7.94 (d, J=2.2 Hz, 1H), 7.69 (dd, J=8.7, 2.2 Hz, 1H), 7.35-7.24 (m, 2H), 7.24-7.04 (m, 3H), 6.98 (d, J=8.6 Hz, 1H), 3.99 (s, 3H), 3.77 (d, J=11.4 Hz, 2H), 2.54 (d, J=6.9 Hz, 2H), 2.35-2.14 (m, 2H), 1.80-1.63 (m, 2H), 1.53-1.24 (m, 3H).

### tert-butyl 4-[5-[(4-benzyl-1-piperidyl)sulfonyl]-2-methoxy-phenyl]piperazine-1-carboxylate (I-011)

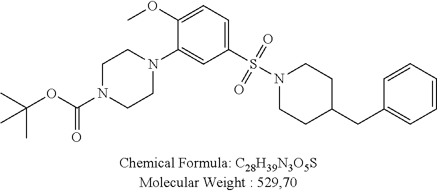 [SMILES: COc1ccc(S(=O)(=O)N2CCC(Cc3ccccc3)CC2)cc1N1CCN(C(=O)OC(C)(C)C)CC1]

According to GP-5, I-011 was obtained as a brown foam in 89% yield using I-010 (1.35 g, 3.18 mmol, 1 equiv.), tert-butyl piperazine-1-carboxylate (889 mg, 4.77 mmol, 1.5 equiv.), Cs₂CO₃ (2.07 g, 6.36 mmol, 2 equiv.), Pd(OAc)₂ (35.7 mg, 0.159 mmol, 0.05 equiv.) and rac-BINAP (119 mg, 0.191 mmol, 0.06 equiv.) in toluene (16 mL) at reflux for 24 h. Purification by FC (cHex/EtOAc=95/5 to 50/50). ¹H NMR (400 MHz, Chloroform-d) δ 7.43 (dd, J=8.5, 2.2 Hz, 1H), 7.35-7.15 (m, 4H), 7.15-7.05 (m, 2H), 6.94 (d, J=8.6 Hz, 1H), 3.95 (s, 3H), 3.76 (d, J=11.5 Hz, 2H), 3.66-3.58 (m, 4H), 3.08-2.98 (m, 4H), 2.53 (d, J=6.6 Hz, 2H), 2.19 (td, J=11.7, 2.4 Hz, 2H), 1.81-1.62 (m, 2H), 1.51 (s, 9H), 1.45-1.30 (m, 3H). MS (ESI⁺): [M+H]⁺ 530.

### I-[5-[(4-benzyl-1-piperidyl)sulfonyl]-2-methoxy-phenyl]piperazine (I-012)

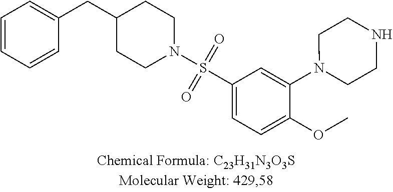 [SMILES: COc1ccc(S(=O)(=O)N2CCC(Cc3ccccc3)CC2)cc1N1CCNCC1]

To a solution of I-011 (2.40 g, 3.35 mmol, 1 equiv.) in DCM (16 mL) at 0 °C, was added HCl (4M solution in dioxane, 8.4 mL, 34 mmol, 10 equiv.). The mixture was stirred at RT for 16 h. The reaction mixture was concentrated under reduced pressure and the residue was partitioned between water and DCM. Na₂CO₃(s) was added portionwise until pH>11. The layers were separated and the aqueous phase was extracted with DCM (2*). The combined organic extracts were dried (Na₂SO₄), filtered and concentrated under reduced pressure. The residue was purified by FC (DCM/MeOH(7N NH₃)=99:1 to 90:10) to afford 1.45 g (100%) of I-012 as a white solid. ¹H NMR (400 MHz, Chloroform-d) δ 7.41 (dd, J=8.5, 2.2 Hz, 1H), 7.32-7.23 (m, 3H), 7.23-7.16 (m, 1H), 7.15-7.05 (m, 2H), 6.93 (d, J=8.5 Hz, 1H), 3.95 (s, 3H), 3.76 (dt, J=12.0, 3.4 Hz, 2H), 3.12-3.00 (m, 8H), 2.53 (d, J=6.6 Hz, 2H), 2.19 (td, J=11.7, 2.4 Hz, 2H), 1.72-1.65 (m, 2H), 1.50-1.30 (m, 3H). MS (ESI⁺): [M+H]⁺ 430.

### Synthesis of Intermediates Required for Final Products 011 to 036

### (2-chloro-4-fluoro-phenyl)-[4-(2-methoxyphenyl)piperazin-1-yl]methanone (I-013)

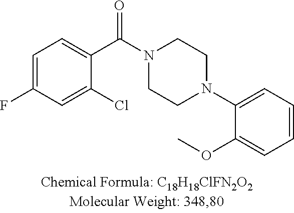 [SMILES: COc1ccccc1N1CCN(C(=O)c2ccc(F)cc2Cl)CC1]

According to GP-1, I-013 was obtained in 85% yield as a colorless oil using 2-chloro-4-fluoro-benzoic acid (5.99 g, 34.3 mmol, 1.2 equiv.), Et₃N (8.0 mL, 57 mmol, 2 equiv.), TBTU (11.0 g, 34.3 mol, 1.2 equiv.) and 1-(2-methoxyphenyl)piperazine (5.50 g. 28.6 mmol, 1 equiv.) in THF (140 mL) at RT for 16 h. The residue was purified by FC (cHex/EtOAc=90/10 to 0/100). ¹H NMR (400 MHz, Chloroform-d) δ 7.35 (dd, J=8.5, 5.9 Hz, 1H), 7.20 (dd, J=8.5, 2.5 Hz, 1H), 7.13-7.03 (m, 2H), 6.98-6.89 (m, 3H), 4.02 (tt, J=12.5, 8.3 Hz, 2H), 3.90 (s, 3H), 3.56-3.37 (m, 2H), 3.26-2.93 (m, 4H). ¹⁹F NMR (376 MHz, Chloroform-d) δ−109.5. MS (ESI⁺): [M+H]⁺ 349/351.

### 3-[4-(2-chloro-4-fluoro-benzoyl)piperazin-1-yl]-4-methoxy-benzenesulfonyl chloride (I-014)

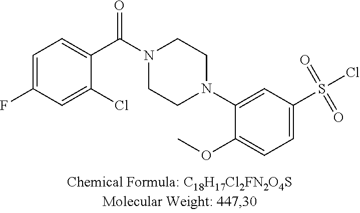 [SMILES: COc1ccc(S(=O)(=O)Cl)cc1N1CCN(C(=O)c2ccc(F)cc2Cl)CC1]

According to GP-3, I-014 was obtained as a white solid in 86% yield using I-013 (5.00 g, 14.3 mmol, 1 equiv.) and HSO₃Cl (19.1 mL, 287 mmol, 20 equiv.) in DCM (72 mL) for 2 h. ¹H NMR (400 MHz, Chloroform-d) δ 7.76 (dd, J=8.7, 2.4 Hz, 1H), 7.49 (d, J=2.4 Hz, 1H), 7.35 (dd, J=8.5, 5.9 Hz, 1H), 7.20 (dd, J=8.5, 2.4 Hz, 1H), 7.10 (td, J=8.2, 2.5 Hz, 1H), 7.03 (d, J=8.8 Hz, 1H), 4.08-3.93 (m, 5H), 3.57-3.36 (m, 2H), 3.30-3.00 (m, 4H). ¹⁹F NMR (376 MHz, Chloroform-d) 6-109.1.

### Synthesis of Intermediate Required for Final Product 037

### 3-[4-(2-chloro-4-fluoro-benzoyl)piperazin-1-yl]-4-methoxy-N-(4,4,4-trifluorobutyl)benzenesulfonamide (I-015)

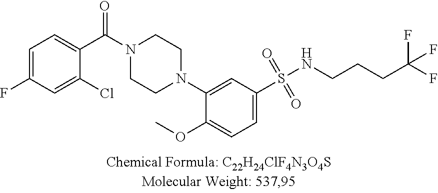 [SMILES: COc1ccc(S(=O)(=O)NCCCC(F)(F)F)cc1N1CCN(C(=O)c2ccc(F)cc2Cl)CC1]

According to GP-4, I-015 was obtained in 64% yield using sulfonyl chloride I-014 (120 mg, 0.268 mmol, 1 equiv.), 4,4,4-Trifluorobutylamine hydrochloride (66 mg, 0.40 mmol, 1.5 equiv.) and iPr₂NEt (140 μL, 0.804 mmol, 3 equiv.) in DCM (2 mL) at RT for 2 h. Purification by FC (cHex/EtOAc=20/80). ¹H NMR (400 MHz, DMSO-d₆) δ 7.58 (dt, J=9.1, 2.2 Hz, 1H), 7.55-7.48 (m, 2H), 7.44 (dt, J=8.6, 2.0 Hz, 1H), 7.34 (tt, J=8.5, 2.1 Hz, 1H), 7.25 (t, J=2.0 Hz, 1H), 7.14 (dd, J=8.7, 1.8 Hz, 1H), 3.88 (s, 3H), 3.84-3.73 (m, 2H), 3.31-3.27 (m, 2H), 3.15-2.91 (m, 4H), 2.78 (q, J=6.7 Hz, 2H), 2.30-2.15 (m, 2H), 1.61-1.50 (m, 2H). ¹⁹F NMR (376 MHz, Chloroform-d) δ−64.8, −110.1. MS (ESI⁺): [M+H]⁺ 538/540.

### Synthesis of Intermediate Required for Final Product 038

### Rac-(2-chloro-4-fluoro-phenyl)-[4-[5-[(3-hydroxy-1-piperidyl)sulfonyl]-2-methoxy-phenyl]piperazin-1-yl]methanone (I-016)

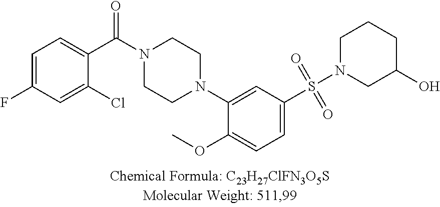 [SMILES: COc1ccc(S(=O)(=O)N2CCCC(O)C2)cc1N1CCN(C(=O)c2ccc(F)cc2Cl)CC1]

According to GP-4, I-016 was obtained in 79% yield using sulfonyl chloride I-014 (150 mg, 0.335 mmol, 1 equiv.), rac-piperidin-3-ol hydrochloride (69 mg, 0.50 mmol, 1.5 equiv.) and iPr₂NEt (175 μL, 1.00 mmol, 3 equiv.) in DCM (2 mL) at RT for 16 h. Purification by FC (DCM/MeOH=98/2 to 90/10). ¹H NMR (400 MHz, Chloroform-d) δ 7.46 (dd, J=8.5, 2.2 Hz, 1H), 7.36-7.30 (m, 1H), 7.22 (d, J=2.2 Hz, 1H), 7.18 (dd, J=8.4, 2.4 Hz, 1H), 7.07 (td, J=8.2, 2.5 Hz, 1H), 6.96 (d, J=8.6 Hz, 1H), 4.02-3.96 (m, 2H), 3.94 (s, 3H), 3.91-3.83 (m, 1H), 3.52-3.44 (m, 1H), 3.43-3.35 (m, 1H), 3.31 (d, J=11.2 Hz, 1H), 3.24-3.07 (m, 4H), 3.02-2.94 (m, 1H), 2.83-2.75 (m, 1H), 2.73-2.66 (m, 1H), 1.90-1.80 (m, 2H), 1.79-1.70 (m, 1H), 1.68-1.56 (m, 1H), 1.40 (dtd, J=12.6, 8.4, 3.9 Hz, 1H). ¹⁹F NMR (376 MHz, Chloroform-d) 6-109.2. MS (ESI⁺): [M+H]⁺ 512/514.

### Synthesis of Intermediate Required for Final Product 039

### (2-chloro-4-fluoro-phenyl)-[4-[5-(3-hydroxypyrrolidin-1-yl)sulfonyl-2-methoxy-phenyl]piperazin-1-yl]methanone (I-017)

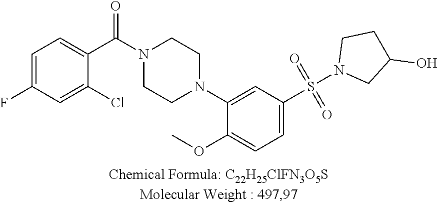 [SMILES: COc1ccc(S(=O)(=O)N2CCC(O)C2)cc1N1CCN(C(=O)c2ccc(F)cc2Cl)CC1]

According to GP-4, I-017 was obtained in 90% yield using sulfonyl chloride I-014 (150 mg, 0.335 mmol, 1 equiv.), rac-pyrrolidin-3-ol (44 mg, 0.50 mmol, 1.5 equiv.) and iPr₂NEt (175 μL, 1.00 mmol, 3 equiv.) in DCM (2 mL) at RT for 16 h. Purification by FC (DCM/MeOH=98/2 to 90/10). ¹H NMR (400 MHz, Chloroform-d) δ 7.54 (dd, J=8.6, 2.1 Hz, 1H), 7.36-7.29 (m, 2H), 7.18 (dd, J=8.5, 2.4 Hz, 1H), 7.07 (td, J=8.3, 2.5 Hz, 1H), 6.96 (d, J=8.6 Hz, 1H), 4.42-4.35 (m, 1H), 3.99 (t, J=5.2 Hz, 2H), 3.94 (s, 3H), 3.52-3.28 (m, 5H), 3.25-3.07 (m, 4H), 2.99 (dd, J=11.2, 6.6 Hz, 1H), 1.97 (dtd, J=13.7, 8.8, 4.9 Hz, 1H), 1.88-1.79 (m, 1H), 1.51 (d, J=4.7 Hz, 1H). ¹⁹F NMR (376 MHz, Chloroform-d) δ−109.2. MS (ESI⁺): [M+H]⁺ 498/500.

### Synthesis of Intermediate Required for Final Product 040

### [4-[5-(2,3,3a,4,6,6a-hexahydro-1H-pyrrolo[3,4-c]pyrrol-5-ylsulfonyl)-2-methoxy-phenyl]piperazin-1-yl]-(2-chloro-4-fluoro-phenyl)methanone (I-018)

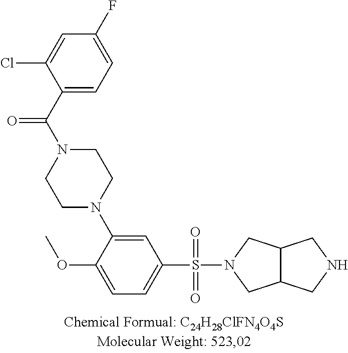 [SMILES: COc1ccc(S(=O)(=O)N2CC3CNCC3C2)cc1N1CCN(C(=O)c2ccc(F)cc2Cl)CC1]

To a solution of 022 (300 mg, 0.481 mmol, 1 equiv.) in DCM (2.4 mL) at 0 °C, was added HCl (4M solution in dioxane, 1.2 mL, 4.8 mmol, 10 equiv.). The mixture was stirred at RT for 2 h. The reaction mixture was concentrated under reduced pressure and the residue was partitioned between water and DCM. K₂CO₃(s) was added portionwise until pH>11. The layers were separated and the aqueous phase was extracted with DCM (2*). The combined organic extracts were dried (Na₂SO₄), filtered and concentrated under reduced pressure to afford 250 mg (99%) of I-018 as a white foam. ¹H NMR (400 MHz, DMSO-d₆) δ 7.58 (dd, J=9.0, 2.4 Hz, 1H), 7.52 (dd, J=8.5, 6.1 Hz, 1H), 7.41 (dd, J=8.6, 2.1 Hz, 1H), 7.34 (td, J=8.5, 2.5 Hz, 1H), 7.18 (d, J=8.6 Hz, 1H), 7.13 (d, J=2.2 Hz, 1H), 3.89 (s, 3H), 3.86-3.69 (m, 2H), 3.30-3.26 (m, 3H), 3.17 (d, J=3.9 Hz, 2H), 3.14-3.06 (m, 4H), 2.98 (t, J=5.0 Hz, 2H), 2.81-2.71 (m, 4H), 2.42 (dd, J=10.8, 2.9 Hz, 2H). ¹⁹F NMR (376 MHz, Chloroform-d) δ−109.2. MS (ESI⁺): [M+H]⁺ 523/525.

### Synthesis of Intermediate Required for Final Product 042

### 3-bromo-4-fluoro-N-methyl-N-propyl-benzenesulfonamide (I-019)

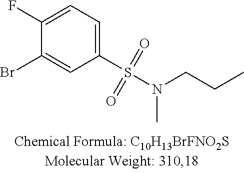 [SMILES: CCCN(C)S(=O)(=O)c1ccc(F)c(Br)c1]

According to GP-4, I-019 was obtained as a yellow oil in 95% yield using 3-bromo-4-fluoro-benzenesulfonyl chloride (200 μL, 1.35 mmol, 1 equiv.), N-methylpropylamine (145 μL, 1.41 mmol, 1.05 equiv.) and triethylamine (281 μL, 2.02 mmol, 1.5 equiv.) in DCM (7 mL) for 1 h at RT. ¹H NMR (400 MHz, Chloroform-d) δ 8.01 (dd, J=6.3, 2.3 Hz, 1H), 7.73 (ddd, J=8.6, 4.5, 2.2 Hz, 1H), 7.33-7.15 (m, 1H), 3.08-2.90 (m, 2H), 2.75 (s, 3H), 1.67-1.55 (m, 2H), 0.94 (t, J=7.4 Hz, 3H). ¹⁹F NMR (376 MHz, Chloroform-d) δ−99.9. MS (ESI⁺): [M+H]⁺ 310/312.

### Synthesis of Intermediate Required for Final Product 043

### [4-(2,3-dihydrobenzofuran-7-yl)piperazin-1-yl]-[4-fluoro-2-(trifluoromethyl)phenyl]methanone (I-020)

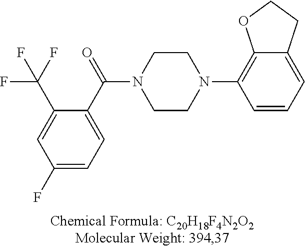 [SMILES: O=C(c1ccc(F)cc1C(F)(F)F)N1CCN(c2cccc3c2OCC3)CC1]

A microwave reaction vial was charged with 7-bromo-2,3-dihydrobenzofuran (300 mg, 1.51 mmol, 1 equiv.), [4-fluoro-2-(trifluoromethyl)phenyl]-piperazin-1-yl-methanone (614 mg, 2.22 mmol, 1.5 equiv.), Pd₂dba₃ (94 mg, 0.10 mmol, 0.07 equiv.), tri(2-tolyl)phosphine (55 mg, 0.18 mmol, 0.12 equiv.) and sodium tert-butylate (212 mg, 2.21 mmol, 1.5 equiv.). The vial was flushed with argon and degassed toluene (10 mL) was added. The vial was sealed and the mixture was stirred at 110 °C for 22 h. The suspension was cooled down to RT, filtered over Celite (EtOAc rinses) and concentrated. The residue was purified by FC (cHex/EtOAc=95/5 to 0/100) to afford 136 mg (23%) of I-020 as a brown foam. ¹H NMR (400 MHz, Chloroform-d) δ 7.42 (dd, J=8.8, 2.5 Hz, 1H), 7.37 (dd, J=8.5, 5.3 Hz, 1H), 7.31 (td, J=8.1, 2.6 Hz, 1H), 6.90 (dd, J=7.4, 1.2 Hz, 1H), 6.81 (t, J=7.6 Hz, 1H), 6.67 (dd, J=7.9, 1.2 Hz, 1H), 4.59 (t, J=8.8 Hz, 2H), 3.97 (qt, J=13.2, 5.1 Hz, 2H), 3.34 (dd, J=5.9, 4.4 Hz, 2H), 3.27-3.11 (m, 4H), 3.10-2.94 (m, 2H). ¹⁹F NMR (376 MHz, Chloroform-d) δ−60.2, −109.4. MS (ESI⁺): [M+H]⁺ 395.

### 7-[4-[4-fluoro-2-(trifluoromethyl)benzoyl]piperazin-1-yl]-2,3-dihydrobenzofuran-5-sulfonyl chloride (I-021)

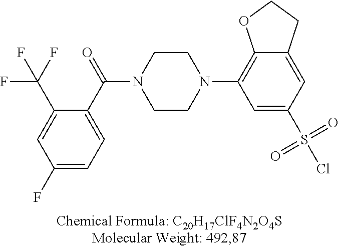 [SMILES: O=C(c1ccc(F)cc1C(F)(F)F)N1CCN(c2cc(S(=O)(=O)Cl)cc3c2OCC3)CC1]

According to GP-3, I-021 was obtained as a white solid in 83% yield using I-020 (135 mg, 0.342 mmol, 1 equiv.) and HSO₃Cl (0.50 mL, 7.5 mmol, 22 equiv.) in DCM (2 mL) for 2 h. ¹H NMR (400 MHz, Chloroform-d) δ 7.58-7.55 (m, 1H), 7.45 (dd, J=8.8, 2.4 Hz, 1H), 7.41-7.32 (m, 2H), 7.32-7.28 (m, 1H), 4.78 (t, J=8.9 Hz, 2H), 3.98 (t, J=5.2 Hz, 2H), 3.43-3.20 (m, 6H), 3.18-2.99 (m, 2H). ¹⁹F NMR (376 MHz, Chloroform-d) δ−60.2, −109.0.

### Synthesis of Intermediates Required for Final Products 044 and 045

### 5-bromo-6-chloro-NN-diethyl-pyridine-3-sulfonamide (I-022)

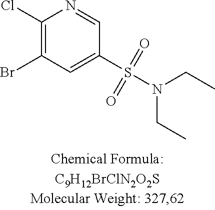 [SMILES: CCN(CC)S(=O)(=O)c1cnc(Cl)c(Br)c1]

According to GP-4, I-022 was obtained as a yellow solid in 100% yield using 5-bromo-6-chloro-pyridine-3-sulfonyl chloride (438 mg, 1.50 mmol, 1.1 equiv.), diethylamine (100 mg, 1.37 mmol, 1 equiv.) and triethylamine (229 μL, 1.64 mmol, 1.2 equiv.) in DCM (6.8 mL) for 1 h at RT. ¹H NMR (400 MHz, Chloroform-d) δ 8.74 (d, J=2.2 Hz, 1H), 8.32 (d, J=2.2 Hz, 1H), 3.31 (q, J=7.2 Hz, 4H), 1.21 (t. J=7.1 Hz, 6H).

### 5-bromo-6-methoxy-N,N-diethyl-pyridine-3-sulfonamide (I-023)

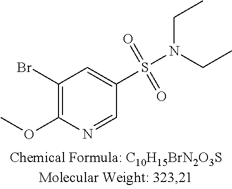 [SMILES: CCN(CC)S(=O)(=O)c1cnc(OC)c(Br)c1]

To a solution of I-022 (150 mg, 0.458 mmol, 1 equiv.) in dry MeOH (1.8 mL) at RT, was added MeONa (124 mg, 2.29 mol, 5 equiv.). The mixture was stirred at RT for 60 h. The RM was partitioned between aq. sat. NH₄Cl and EtOAc. The layers were separated and the aqueous phase was extracted with EtOAc. The combined organic extracts were washed (brine), dried (Na₂SO₄), filtered and concentrated under reduced pressure to afford 138 mg (93%) of I-023 as light yellow solid. ¹H NMR (400 MHz, Chloroform-d) δ 8.55 (d, J=2.2 Hz, 1H), 8.18 (d, J=2.2 Hz, 1H), 4.10 (s, 3H), 3.28 (q, J=7.2 Hz, 4H), 1.20 (t, J=7.1 Hz, 6H).

### Synthesis of Intermediates Required for Final Products 046 and 047

### 3-bromo-4-hydroxy-N-methyl-N-propyl-benzenesulfonamide (I-024)

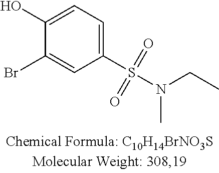

To a solution of I-007 (200 mg, 0.620 mmol, 1 equiv.) in DCM (7.7 mL) at 0 °C, was added BBr₃ (IM solution in DCM, 1.86 mL, 1.86 mmol, 2 equiv.). The mixture was stirred at RT for 48 h.

After cooling down to 0 °C, water was added dropwise (exotherm). The layers were separated and the aqueous phase was extracted with DCM (2*). The combined organic extracts were dried (Na₂SO₄), filtered and concentrated under reduced pressure to afford 214 mg (quantitative) of I-024 as an orange oil. ¹H NMR (400 MHz, Chloroform-d) δ (ppm) 7.95 (d, J=2.2 Hz, 1H), 7.74 (dd, J=8.6 and 2.2 Hz, 1H), 7.14 (d, J=8.6 Hz, 1H), 6.05 (br s, 1H), 3.02-2.95 (m, 2H), 2.75 (s, 3H), 1.65-1.52 (m, 2H), 0.95 (t, J=7.4 Hz, 3H). MS (ESI⁺): [M+H]⁺ 308/310.

### 3-bromo-4-(2-methoxyethoxy)-N-methyl-N-propyl-benzenesulfonamide (I-025)

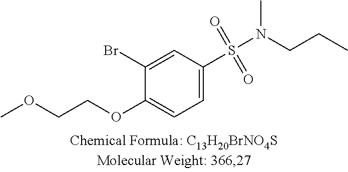 [SMILES: CCCN(C)S(=O)(=O)c1ccc(OCCOC)c(Br)c1]

To a solution of I-024 (191 mg, 0.620 mmol, 1 equiv.) in DMF (2.5 mL) at RT, were added K₂CO₃ (257 mg, 1.86 mmol, 3 equiv.) and 2-bromomethyl methyl ether (129 mg, 0.930 mmol, 1.5 equiv.). The mixture was stirred at 50 °C for 16 h. The reaction mixture was partitioned between aq. sat. NH₄Cl and EtOAc. The layers were separated and the aqueous phase was extracted with EtOAc (2*). The combined organic extracts were washed (sat. aq. NH₄Cl, sat. aq. NaHCO₃, brine), dried (Na₂SO₄), filtered and concentrated under reduced pressure. The residue was purified by FC (cHex/EtOAc=90/10 to 50/50) to afford 200 mg (88%) of I-025 as a yellow oil. ¹H NMR (400 MHz, Chloroform-d) δ 7.98 (d, J=2.2 Hz, 1H), 7.71 (dd, J=8.6, 2.3 Hz, 1H), 7.02 (d, J=8.7 Hz, 1H), 4.32-4.22 (m, 2H), 3.91-3.83 (m, 2H), 3.51 (s, 3H), 2.98 (dd, J=8.0, 6.5 Hz, 2H), 2.74 (s, 3H), 1.70-1.50 (m, 2H), 0.95 (t, J=7.4 Hz, 3H). MS (ESI⁺): [M+H]⁺ 366/368.

### 4-benzyloxy-3-bromo-N-methyl-N-propyl-benzenesulfonamide (I-026)

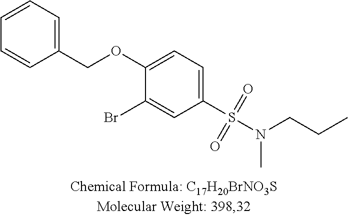 [SMILES: CCCN(C)S(=O)(=O)c1ccc(OCc2ccccc2)c(Br)c1]

To a solution of I-024 (315 mg, 0.961 mmol, 1 equiv.) in DMF (10 mL) at RT, were added K₂CO₃ (266 mg, 1.92 mmol, 2 equiv.) and benzyl bromide (114 μL, 0.961 mmol, 1 equiv). The mixture was stirred at RT for 16 h. The reaction mixture was partitioned between aq. sat. NH₄Cl and EtOAc. The layers were separated and the aqueous phase was extracted with EtOAc (2*). The combined organic extracts were washed (sat. aq. NH₄Cl, sat. aq. NaHCO₃, brine), dried (Na₂SO₄), filtered and concentrated under reduced pressure. The residue was purified by FC (cHex/EtOAc=100/0 to 50/50) to afford 330 mg (86%) of I-026 as a yellow oil. ¹H NMR (400 MHz, Chloroform-d) δ 7.99 (d, J=2.3 Hz, 1H), 7.67 (dd, J=8.6, 2.2 Hz, 1H), 7.49-7.44 (m, 2H), 7.44-7.38 (m, 2H), 7.38-7.31 (m, 1H), 7.01 (d, J=8.7 Hz, 1H), 5.23 (s, 2H), 3.05-2.91 (m, 2H), 2.72 (s, 3H), 1.67-1.49 (m, 2H), 0.93 (t, J=7.4 Hz, 3H). MS (ESI⁺): [M+H]⁺ 398/400.

### Synthesis of Intermediates Required for Piperazine 1-027

### tert-butyl 4-(2-chloro-4-fluoro-benzoyl)piperazine-1-carboxylate (Boc-1-027)

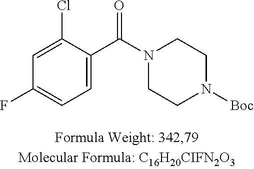 [SMILES: CC(C)(C)OC(=O)N1CCN(C(=O)c2ccc(F)cc2Cl)CC1]

To a solution of 2-chloro-4-fluorobenzoic acid (5.00 g, 28.6 mmol, 1 equiv.) in THF (89 mL) at RT, was added TBTU (9.20 g, 28.6 mmol, 1 equiv.). The mixture was stirred 15 min at RT, and a solution of tert-butyl piperazine-1-carboxylate (6.40 g, 34.4 mmol, 1.2 equiv.) and Et₃N (6.0 mL, 43 mmol, 1.5 equiv.) in THF (30 mL) was added dropwise. The mixture was stirred at RT for 60 h. The reaction mixture was concentrated under reduced pressure. The reaction mixture was partitioned between 1N HCl and EtOAc. The layers were separated and the aqueous phase was extracted with EtOAc(2*). The combined organic extracts were washed (aq. 1N HCl, aq. sat. NaHCO₃, brine), dried (Na₂SO₄), filtered and concentrated under reduced pressure to afford 10.2 g (100%) of tert-butyl 4-(2-chloro-4-fluoro-benzoyl)piperazine-1-carboxylate (Boc-I-027) as an orange oil. ¹H NMR (400 MHz, Chloroform-d) δ 7.32-7.29 (m, 1H), 7.18 (dd, J=8.5, 2.5 Hz, 1H), 7.07 (td, J=8.3, 2.5 Hz, 1H), 3.92-3.79 (m, 1H), 3.73 (dt, J=13.2, 5.7 Hz, 1H), 3.63-3.50 (m, 2H), 3.48-3.34 (m, 2H), 3.34-3.10 (m, 2H), 1.48 (s, 9H). MS (ESI⁺): [M+H]⁺ 343.1/345.1.

### (2-chloro-4-fluoro-phenyl)-piperazin-1-yl-methanone (I-027)

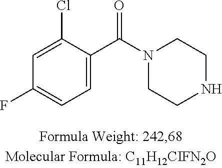 [SMILES: O=C(c1ccc(F)cc1Cl)N1CCNCC1]

To a solution of crude tert-butyl 4-(2-chloro-4-fluoro-benzoyl)piperazine-1-carboxylate Boc-I-027 (10.2 g, 28.6 mmol, 1 equiv.) in dioxane (37 mL) at RT, was added HCl (4M solution in dioxane, 37 mL, 148 mmol, 5 equiv.). The mixture was stirred at RT for 16 h. The reaction mixture was concentrated under reduced pressure and the residue was partitioned between water and DCM. K₂CO₃(s) was added portionwise until pH>11. The layers were separated and the aqueous phase was extracted with DCM (2*). The combined organic extracts were dried (Na₂SO₄), filtered and concentrated under reduced pressure. The residue was purified by FC (80 g column, dry load, DCM/MeOH=98/2 to 80/20) to afford 5.24 g (72%) of (2-chloro-4-fluoro-phenyl)-piperazin-1-yl-methanone I-027 as an orange solid. ¹H NMR (400 MHz, Chloroform-d) δ 7.41 (dd, J=8.8, 2.4 Hz, 1H), 7.32 (qd, J=8.4, 5.6 Hz, 2H), 3.87-3.69 (m, 2H), 3.15 (t, J=5.1 Hz, 2H), 2.94 (t, J=5.2 Hz, 2H), 2.80-2.70 (m, 2H). MS (ESI⁺): [M+H]⁺ 243.1/245.1.

### Synthesis of Intermediates Required for Final Products 054 & 55

### 1-bromo-2-(methoxymethoxy)-3-methyl-benzene (I-028)

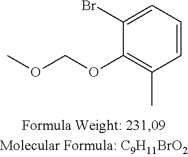

To a solution of 2-bromo-6-methyl-phenol (1.00 g, 5.35 mmol, 1 equiv.) and iPr₂NEt (1.4 mL, 8.1 mmol, 1.5 equiv.) in DCM (21 mL) at RT, was added chloromethyl methyl ether (0.49 mL, 6.4 mmol, 1.2 equiv.). The mixture was stirred at RT for 60 h. 1N HCl was added. The layers were separated and the aqueous phase was extracted with DCM. The combined organic extracts 20 were dried (Na₂SO₄), filtered and concentrated under reduced pressure to afford 1.22 g of I-028 (99%) as a colorless oil. ¹H NMR (400 MHz, Chloroform-d) δ 7.42 (ddd, J=8.0, 1.6, 0.7 Hz, 1H), 7.15 (ddd, J=7.6, 1.6, 0.8 Hz, 1H), 6.92 (t, J=7.8 Hz, 1H), 5.11 (s, 2H), 3.68 (s, 3H), 2.39 (s, 3H).

### (2-chloro-4-fluoro-phenyl)-[4-[2-(methoxymethoxy)-3-methyl-phenyl]piperazin-1-yl]methanone (I-029)

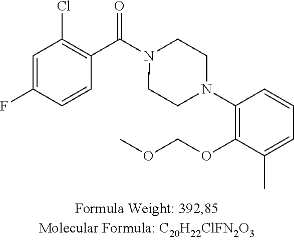 [SMILES: COCOc1c(C)cccc1N1CCN(C(=O)c2ccc(F)cc2Cl)CC1]

According to GP-5, I-029 was obtained as an oil in 44% yield using I-028 (400 mg, 1.73 mmol, 1 equiv.), piperazine I-027 (504 mg, 2.08 mmol, 1.2 equiv.), Cs₂CO₃ (1.69 g, 5.19 mmol, 3 equiv.), Pd(OAc)₂ (38.9 mg, 173 μmol, 0.1 equiv.) and rac-BINAP (162 mg, 260 μmol, 0.15 equiv.) in toluene (8.7 mL) at reflux for 2 h. Purification by FC (cHex/EtOAc=95/5 to 50/50). ¹H NMR (400 MHz, Chloroform-d) δ 7.32 (dd, J=8.5, 5.9 Hz, 1H), 7.18 (dd, J=8.4, 2.4 Hz, 1H), 7.07 (td, J=8.2, 2.5 Hz, 1H), 6.98 (t, J=7.7 Hz, 1H), 6.94-6.86 (m, 1H), 6.79 (dd, J=8.0, 1.7 Hz, 1H), 5.17 (s, 2H), 3.96 (t, J=5.1 Hz, 2H), 3.60 (s, 3H), 3.50-3.28 (m, 2H), 3.28-3.02 (m, 3H), 3.02-2.88 (m, 1H), 2.32 (s, 3H). ¹⁹F NMR (376 MHz, Chloroform-d) δ−109.4. MS (ESI⁺): [M+H]⁺ 393.1/395.1.

### 3-[4-(2-chloro-4-fluoro-benzoyl)piperazin-1-yl]-4-hydroxy-5-methyl-benzenesulfonyl chloride (I-030)

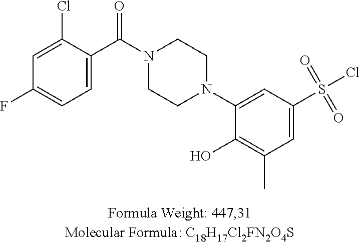 [SMILES: Cc1cc(S(=O)(=O)Cl)cc(N2CCN(C(=O)c3ccc(F)cc3Cl)CC2)c1O]

According to GP-3, I-030 was obtained as a yellow solid in 50% yield using I-029 (300 mg, 0.748 mmol, 1 equiv.) and HSO₃Cl (1.2 mL, 19 mmol, 25 equiv.) in DCM (3 mL) at RT for 1 h. The material was directly engaged in the next step

### Synthesis of Intermediates Required for Final Products 057 to 59

### 1-bromo-3-chloro-2-(methoxymethoxy)benzene (I-031)

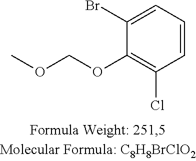

To a solution of 2-bromo-6-chlorophenol (3.17 g, 10.5 mmol, 1 equiv.) and iPr₂NEt (3.73 mL, 15.7 mmol, 1.5 equiv.) in DCM (21 mL) at RT, was added chloromethyl methyl ether (0.95 mL, 13 mmol, 1.2 equiv.). The mixture was stirred at RT for 16 h. The volatiles were removed under reduced pressure. EtOAc was added and the organic solution was washed (1N HCl, brine), dried (Na₂SO₄), filtered and concentrated under reduced pressure. The residue was purified by FC (cHex/EtOAc=95/5) to afford 2.59 g of I-031(98%) as a colorless oil. ¹H NMR (400 MHz, Chloroform-d) δ 7.50 (dd, J=8.0, 1.6 Hz, 1H), 7.37 (dd, J=8.1, 1.5 Hz, 1H), 6.97 (t, J=8.0 Hz, 1H), 5.21 (s, 2H), 3.74 (s, 3H).

### (2-chloro-4-fluoro-phenyl)-[4-[3-chloro-2-(methoxymethoxy)phenyl]piperazin-1-yl]methanone (I-032)

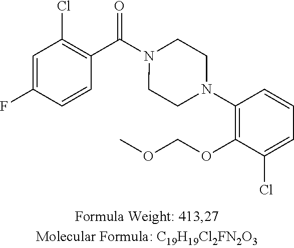 [SMILES: COCOc1c(Cl)cccc1N1CCN(C(=O)c2ccc(F)cc2Cl)CC1]

According to GP-5, I-032 was obtained as a white foam in 62% yield using aryl bromide I-031 (1.23 g, 4.89 mmol, 1 equiv.), piperazine I-027 (1.42 g, 5.87 mmol, 1.2 equiv.), Cs₂CO₃ (4.78 g, 14.7 mmol, 3 equiv.), Pd(OAc)₂ (110 mg, 489 μmol, 0.1 equiv.) and rac-BINAP (365 mg, 587 μmol, 0.12 equiv.) in toluene (24.5 mL) at reflux for 16 h. Purification by FC (cHex/EtOAc=95/5 to 50/50). ¹H NMR (400 MHz, Chloroform-d) δ 7.30 (dd, J=8.5, 5.9 Hz, 1H), 7.18 (dd, J=8.4, 2.4 Hz, 1H), 7.12-7.03 (m, 2H), 7.00 (t, J=8.1 Hz, 1H), 6.83 (dd, J=8.1, 1.5 Hz, 1H), 5.22 (s, 2H), 4.01-3.86 (m, 2H), 3.66 (s, 3H), 3.50-3.29 (m, 2H), 3.25-2.91 (m, 4H). ¹⁹F NMR (376 MHz, Chloroform-d) δ−109.2. MS (ESI⁺): [M+H]⁺ 413.0/415.0/417.0.

### (2-chloro-4-fluoro-phenyl)-[4-(3-chloro-2-hydroxy-phenyl)piperazin-1-yl]methanone (I-033)

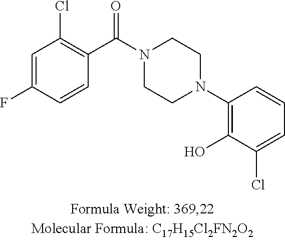 [SMILES: O=C(c1ccc(F)cc1Cl)N1CCN(c2cccc(Cl)c2O)CC1]

To a solution of I-032 (1.26 g, 3.05 mmol, 1 equiv.) in DCM (15 mL) at RT, was added CF₃CO₂H (4.7 mL, 61 mmol, 20 equiv.). The mixture was stirred at RT for 1 h. It was concentrated under reduced pressure and the residue was azeotroped with toluene. The residue was purified by FC (cHex/EtOAc=95/5 to 20/80) to afford 1.12 g of I-033 (97%) as a white foam. ¹H NMR (400 MHz, Chloroform-d) δ 7.35 (dd, J=8.5, 5.8 Hz, 1H), 7.24-7.16 (m, 2H), 7.14-7.02 (m, 2H), 6.86 (t, J=8.1 Hz, 1H), 6.26 (s, 1H), 4.22-4.06 (m, 1H), 4.06-3.93 (m, 1H), 3.59-3.38 (m, 2H), 3.28-2.86 (m, 4H). ¹⁹F NMR (376 MHz, Chloroform-d) 6-108.1. MS (ESI⁺): [M+H]⁺ 369.0/371.0/373.0.

### 3-chloro-5-[4-(2-chloro-4-fluoro-benzoyl)piperazin-1-yl]-4-hydroxy-benzenesulfonyl chloride (I-034)

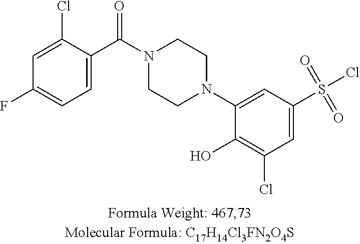 [SMILES: O=C(c1ccc(F)cc1Cl)N1CCN(c2cc(S(=O)(=O)Cl)cc(Cl)c2O)CC1]

According to GP-3, I-034 was obtained as a beige solid in 80% yield using I-033 (500 mg, 1.35 mmol, 1 equiv.) and HSO₃Cl (2.3 mL, 34 mmol, 25 equiv.) in DCM (7 mL) at reflux for 4 h. ¹H NMR (400 MHz, Chloroform-d) δ 7.88 (d, J=2.2 Hz, 1H), 7.62 (d, J=2.2 Hz, 1H), 7.34 (dd, J=8.5, 5.8 Hz, 1H), 7.19 (dd, J=8.4, 2.4 Hz, 1H), 7.09 (td, J=8.2, 2.5 Hz, 1H), 5.75 (br s, 1H), 4.03 (t, J=5.1 Hz, 2H), 3.61-3.34 (m, 2H), 3.19-2.85 (m, 4H). ¹⁹F NMR (376 MHz, Chloroform-d) δ−108.5.

### Synthesis of Final Compounds

### Synthetic Methods for Final Compounds

The synthetic protocols for the final compounds are presented on the following Table 2.

TABLE 2

| Cpd | Synthetic protocols | Purity |
|---|---|---|
| 001 | According to GP-2, 001 was obtained in 68% yield using 2-chloro- | >95% |
| | 4-fluoro-benzoic acid (75 mg, 0.43 mmol, 2 equiv.), iPr₂NEt (98 μL, | |
| | 0.56 mmol, 2.7 equiv.), TPTU (127 mg, 428 μmol, 2 equiv.) and | |
| | piperazine I-003 (70 mg, 0.21 mmol, 1 equiv.) in DCE/MeCN | |
| | (3 mL) at RT for 16 h. Purification by semi-preparative HPLC. | |
| 002 | According to GP-2, 002 was obtained in 69% yield using 2-chloro- | >95% |
| | 4,5-difluorobenzoic acid (59 mg, 0.31 mmol, 2 equiv.), iPr₂NEt | |
| | (98 μL, 0.56 mmol, 3.7 equiv.), TPTU (91 mg, 0.31 mmol, 2 equiv.) | |
| | and piperazine I-003 (50 mg, 0.15 mmol, 1 equiv.) in DCE/MeCN | |
| | (3 mL) at RT for 16 h. Purification by semi-preparative HPLC. | |
| 003 | According to GP-2, 003 was obtained in 5% yield using 2,4- | >95% |
| | dichlorobenzoic acid (75 mg, 0.43 mmol, 2 equiv.), iPr₂NEt (98 μL, | |
| | 0.56 mmol, 2.5 equiv.), TPTU (127 mg, 428 μmol, 2 equiv.) and | |
| | piperazine I-006 (70 mg, 0.22 mmol, 1 equiv.) in DCE/MeCN | |
| | (3 mL) at RT for 16 h. Purification by semi-preparative HPLC. | |
| 004 | According to GP-2, 004 was obtained in 7% yield using 2-chloro- | >95% |
| | 4-fluoro-benzoic acid (75 mg, 0.43 mmol, 2 equiv.), iPr₂NEt (98 μL, | |
| | 0.56 mmol, 2.5 equiv.), TPTU (127 mg, 428 μmol, 2 equiv.) and | |
| | piperazine I-006 (70 mg, 0.22 mmol, 1 equiv.) in DCE/MeCN | |
| | (3 mL) at RT for 16 h. Purification by semi-preparative HPLC. | |
| 005 | According to GP-2, 005 was obtained in 57% yield using 2-chloro- | >95% |
| | 4,5-difluorobenzoic acid (59 mg, 0.31 mmol, 2 equiv.), iPr₂NEt | |
| | (98 μL, 0.56 mmol, 3.7 equiv.), TPTU (91 mg, 0.31 mmol, 2 equiv.) | |
| | and piperazine I-009 (50 mg, 0.15 mmol, 1 equiv.) in DCE/MeCN | |
| | (3 mL) at RT for 16 h. Purification by semi-preparative HPLC. | |
| 006 | According to GP-2, 006 was obtained in 58% yield using 2- | >95% |
| | (difluoromethyl)-4-fluoro-benzoic acid (35 mg, 0.18 mmol, 1.2 | |
| | equiv.), iPr₂NEt (53 μL, 0.31 mmol, 2 equiv.), TPTU (91 mg, | |
| | 0.31 mmol, 2 equiv.) and piperazine I-009 (50 mg, 0.15 mmol, 1 | |
| | equiv.) in DCE/MeCN (3 mL) at RT for 16 h. Purification PTLC | |
| | (cHex/EtOAc = 50/50). | |
| 007 | According to GP-2, 007 was obtained in 58% yield using 2- | >95% |
| | (difluoromethyl)benzoic acid (44 mg, 0.24 mmol, 2 equiv.), iPr₂NEt | |
| | (98 μL, 0.56 mmol, 4.5 equiv.), TPTU (39 mg, 0.24 mmol, 2 equiv.) | |
| | and piperazine I-012 (50 mg, 0.12 mmol, 1 equiv.) in DCE/MeCN | |
| | (3 mL) at RT for 16 h. Purification by semi-preparative HPLC. | |
| 008 | According to GP-2, 008 was obtained in 57% yield using 2-chloro- | >95% |
| | 4,5-difluorobenzoic acid (25 mg, 0.14 mmol, 2 equiv.), iPr₂NEt | |
| | (65 μL, 0.37 mmol, 4 equiv.), TPTU (42 mg, 0.14 mmol, 1.5 equiv.) | |
| | and piperazine I-012 (40 mg, 93 μmol, 1 equiv.) in DCE/MeCN | |
| | (3 mL) at RT for 16 h. Purification by semi-preparative HPLC. | |
| 009 | According to GP-2, 009 was obtained in 57% yield using 2-chloro- | >95% |
| | 4,5-difluorobenzoic acid (45 mg, 0.24 mmol, 2 equiv.), iPr₂NEt | |
| | (98 μL, 0.56 mmol, 4.5 equiv.), TPTU (69 mg, 0.24 mmol, 2 equiv.) | |
| | and piperazine I-012 (50 mg, 0.12 mmol, 1 equiv.) in DCE/MeCN | |
| | (3 mL) at RT for 16 h. Purification by semi-preparative HPLC. | |
| 010 | According to GP-2, 010 was obtained in 57% yield using 2- | >95% |
| | (difluoromethyl)-4-fluoro-benzoic acid (13 mg, 0.070 mmol, 1.2 | |
| | equiv.), iPr₂NEt (20 μL, 0.12 mmol, 2 equiv.), TPTU (21 mg, | |
| | 0.070 mmol, 1.2 equiv.) and piperazine I-012 (25 mg, 0.058 mmol, 1 | |
| | equiv.) in DCE/MeCN (1 mL) at RT for 16 h. Purification PTLC | |
| | (cHex/EtOAc = 50/50). | |
| 011 | According to GP-4, 011 was obtained in 82% yield using sulfonyl | >95% |
| | chloride I-014 (50 mg, 0.12 mmol, 1 equiv.), 4-hydroxy-4- | |
| | phenylpiperidine (30 mg, 0.17 mmol, 1.5 equiv.) and iPr₂NEt (39 μL, | |
| | 0.22 mmol, 2 equiv.) in DCM (0.5 mL) at RT for 1 h. Purification by | |
| | PTLC (cHex/EtOAc = 50/50). | |
| 012 | According to GP-4, 012 was obtained in 38% yield using sulfonyl | >95% |
| | chloride I-014 (70 mg, 0.16 mmol, 1 equiv.), 4-hydroxy-4- | |
| | phenylpiperidine (60 mg, 0.32 mmol, 2 equiv.) and iPr₂NEt (110 μL, | |
| | 0.63 mmol, 4 equiv.) in DCM (1 mL) at RT for 16 h. Purification by | |
| | semi-preparative HPLC. | |
| 013 | According to GP-4, 013 was obtained in 96% yield using sulfonyl | >95% |
| | chloride I-014 (50 mg, 0.11 mmol, 1 equiv.), N-benzylpiperazine | |
| | (39 μL, 0.22 mmol, 2 equiv.) and iPr₂NEt (39 μL, 0.63 mmol, 2 | |
| | equiv.) in DCM (0.6 mL) at RT for 2 h. Purification by PTLC | |
| | (EtOAc). | |
| 014 | According to GP-4, 014 was obtained in 62% yield using sulfonyl | >95% |
| | chloride I-014 (70 mg, 0.16 mmol, 1 equiv.), 1-cyclobutyl-N- | |
| | methyl-methanamine (31 μL, 0.32 mmol, 2 equiv.) and iPr₂NEt | |
| | (110 μL, 0.63 mmol, 4 equiv.) in DCM (1 mL) at RT for 16 h. | |
| | Purification by semi-preparative HPLC. | |
| 015 | According to GP-4, 015 was obtained in 65% yield using sulfonyl | >95% |
| | chloride I-014 (70 mg, 0.16 mmol, 1 equiv.), 1-cyclohexyl-N- | |
| | methyl-methanamine (40 mg, 0.32 mmol, 2 equiv.) and iPr₂NEt | |
| | (110 μL, 0.63 mmol, 4 equiv.) in DCM (1 mL) at RT for 16 h. | |
| | Purification by semi-preparative HPLC. | |
| 016 | According to GP-4, 016 was obtained in 63% yield using sulfonyl | >95% |
| | chloride I-014 (70 mg, 0.16 mmol, 1 equiv.), N- | |
| | methylcyclopentanamine (31 mg, 0.32 mmol, 2 equiv.) and iPr₂NEt | |
| | (110 μL, 0.63 mmol, 4 equiv.) in DCM (1 mL) at RT for 16 h. | |
| | Purification by semi-preparative HPLC. | |
| 017 | According to GP-4, 017 was obtained in 56% yield using sulfonyl | >95% |
| | chloride I-014 (70 mg, 0.16 mmol, 1 equiv.), N- | |
| | methylcyclohexanamine (41 μL, 0.32 mmol, 2 equiv.) and iPr₂NEt | |
| | (110 μL, 0.63 mmol, 4 equiv.) in DCM (1 mL) at RT for 16 h. | |
| | Purification by semi-preparative HPLC. | |
| 018 | According to GP-4, 018 was obtained in 59% yield using sulfonyl | >95% |
| | chloride I-014 (50 mg, 0.11 mmol, 1 equiv.), N,3-Dimethyl-3- | |
| | oxetanamine hydrochloride (23 mg, 0.17 mmol, 1.5 equiv.) and | |
| | iPr₂NEt (58 μL, 0.34 mmol, 3 equiv.) in DCM (1 mL) at RT for 5 h. | |
| | Purification by FC (cHex/EtOAc = 70/30 to 0/100). | |
| 019 | According to GP-4, 019 was obtained in 48% yield using sulfonyl | >95% |
| | chloride I-014 (70 mg, 0.16 mmol, 1 equiv.), N-methylbenzylamine | |
| | (40 μL, 0.32 mmol, 2 equiv.) and iPr₂NEt (110 μL, 0.63 mmol, 4 | |
| | equiv.) in DCM (1 mL) at RT for 16 h. Purification by semi- | |
| | preparative HPLC. | |
| 020 | According to GP-4, 020 was obtained in 57% yield using sulfonyl | >95% |
| | chloride I-014 (70 mg, 0.16 mmol, 1 equiv.), N-methyl-3- | |
| | chlorobenzylamine (45 μL, 0.32 mmol, 2 equiv.) and iPr₂NEt | |
| | (110 μL, 0.63 mmol, 4 equiv.) in DCM (1 mL) at RT for 16 h. | |
| | Purification by semi-preparative HPLC. | |
| 021 | According to GP-4, 021 was obtained in 51% yield using sulfonyl | >95% |
| | chloride I-014 (70 mg, 0.16 mmol, 1 equiv.), N-methyl-4- | |
| | fluorobenzylamine (41 μL, 0.32 mmol, 2 equiv.) and iPr₂NEt | |
| | (110 μL, 0.63 mmol, 4 equiv.) in DCM (1 mL) at RT for 16 h. | |
| | Purification by semi-preparative HPLC. | |
| 022 | According to GP-4, 022 was obtained in 82% yield using sulfonyl | >95% |
| | chloride I-014 (300 mg, 0.67 mmol, 1 equiv.), tert-butyl | |
| | 2,3,3a,4,6,6a-hexahydro-1H-pyrrolo[3,4-c]pyrrole-5-carboxylate | |
| | (157 mg, 0.74 mmol, 1.1 equiv.) and Et₃N (187 μL, 1.3 mmol, 2 | |
| | equiv.) in DCM (3.5 mL) at RT for 1 h. Purification by FC | |
| | (cHex/EtOAc = 90/10 to 0/100). | |
| 023 | According to GP-4, 023 was obtained in 100% yield using sulfonyl | >95% |
| | chloride I-014 (114 mg, 0.25 mmol, 1.1 equiv.), 4- | |
| | benzylidenepiperidine hydrochloride (48 mg, 0.23 mmol, 1 equiv.) | |
| | and Et₃N (96 μL, 0.70 mmol, 3 equiv.) in DCM (3.5 mL) at RT for | |
| | 16 h. Purification by PTLC (cHex/EtOAc = 50/50). | |
| 024 | According to GP-4, 024 was obtained in 65% yield using sulfonyl | >95% |
| | chloride I-014 (70 mg, 0.16 mmol, 1 equiv.), 4-(2- | |
| | phenylethyl)piperidine (59 mg, 0.32 mmol, 2 equiv.) and iPr₂NEt | |
| | (110 μL, 0.63 mmol, 4 equiv.) in DCM (1 mL) at RT for 16 h. | |
| | Purification by semi-preparative HPLC. | |
| 025 | According to GP-4, 025 was obtained in 55% yield using sulfonyl | >95% |
| | chloride I-014 (70 mg, 0.16 mol, 1 equiv.), 4-phenylpiperidine | |
| | (51 mg, 0.32 mmol, 2 equiv.) and iPr₂NEt (110 μL, 0.63 mmol, 4 | |
| | equiv.) in DCM (1 mL) at RT for 16 h. Purification by semi- | |
| | preparative HPLC. | |
| 026 | According to GP-4, 026 was obtained in 57% yield using sulfonyl | >95% |
| | chloride I-014 (70 mg, 0.16 mol, 1 equiv.), rac-3-phenylpiperidine | |
| | (51 mg, 0.32 mmol, 2 equiv.) and iPr₂NEt (110 μL, 0.63 mmol, 4 | |
| | equiv.) in DCM (1 mL) at RT for 16 h. Purification by semi- | |
| | preparative HPLC. | |
| 027 | According to GP-4, 027 was obtained in 81% yield using sulfonyl | >95% |
| | chloride I-014 (50 mg, 0.11 mol, 1 equiv.), 3-benzylpiperidine | |
| | (24 μL, 0.13 mmol, 1.2 equiv.) and Et₃N (58 μL, 0.34 mmol, 3 equiv.) | |
| | in DCM (0.6 mL) at RT for 16 h. Purification by PTLC | |
| | (cHex/EtOAc = 50/50). | |
| 028 | According to GP-4, 028 was obtained in 52% yield using sulfonyl | >95% |
| | chloride I-014 (70 mg, 0.16 mol, 1 equiv.), 3-phenylpyrrolidine | |
| | (46 mg, 0.32 mmol, 2 equiv.) and iPr₂NEt (110 μL, 0.63 mmol, 4 | |
| | equiv.) in DCM (1 mL) at RT for 16 h. Purification by semi- | |
| | preparative HPLC. | |
| 029 | According to GP-4, 023 was obtained in 73% yield using sulfonyl | >95% |
| | chloride I-014 (153 mg, 0.34 mmol, 1.1 equiv.), 3-benzylpyrrolidine | |
| | (50 mg, 0.31 mmol, 1 equiv.) and iPr₂NEt (216 μL, 1.24 mmol, 4 | |
| | equiv.) in DCM (1.5 mL) at RT for 16 h. Purification by PTLC | |
| | (cHex/EtOAc = 40/60). | |
| 030 | According to GP-4, 030 was obtained in 60% yield using sulfonyl | >95% |
| | chloride I-014 (70 mg, 0.16 mol, 1 equiv.), 4-phenylpiperidine | |
| | (39 μL, 0.32 mmol, 2 equiv.) and iPr₂NEt (110 μL, 0.63 mmol, 4 | |
| | equiv.) in DCM (1 mL) at RT for 16 h. Purification by semi- | |
| | preparative HPLC. | |
| 031 | According to GP-4, 031 was obtained in 86% yield using sulfonyl | >95% |
| | chloride I-014 (50 mg, 0.12 mmol, 1 equiv.), 4- | |
| | trifluoromethylpiperidine hydrochloride (32 mg, 0.17 mmol, 1.5 | |
| | equiv.) and iPr₂NEt (58 μL, 0.34 mmol, 2 equiv.) in DCM (0.6 mL) | |
| | at RT for 3 h. Purification by PTLC (cHex/EtOAc = 20/80). | |
| 032 | According to GP-4, 032 was obtained in 67% yield using sulfonyl | >95% |
| | chloride I-014 (50 mg, 0.12 mmol, 1 equiv.), tert-butylamine (18 μL, | |
| | 0.17 mmol, 1.5 equiv.) and iPr₂NEt (40 μL, 0.23 mmol, 2 equiv.) in | |
| | DCM (1 mL) at RT for 3 h. Purification by PTLC (DCM/MeOH = | |
| | 99/1). | |
| 033 | According to GP-4, 033 was obtained in 52% yield using sulfonyl | >95% |
| | chloride I-014 (50 mg, 0.12 mmol, 1 equiv.), N,2-dimethylpropan- | |
| | 2-amine (20 μL, 0.17 mmol, 1.5 equiv.) and iPr₂NEt (40 μL, | |
| | 0.23 mmol, 2 equiv.) in DCM (0.6 mL) at RT for 3 h. Purification by | |
| | PTLC (DCM/MeOH = 98/2). | |
| 034 | According to GP-4, 034 was obtained in 75% yield using sulfonyl | >95% |
| | chloride I-014 (50 mg, 0.12 mmol, 1 equiv.), 2-adamantanamine | |
| | hydrochloride (25 mg, 0.17 mmol, 1.5 equiv.) and iPr₂NEt (58 μL, | |
| | 0.34 mmol, 3 equiv.) in DCM (0.6 mL) at RT for 3 h. Purification by | |
| | FC (cHex/EtOAc = 95/5 to 0/100). | |
| 035 | According to GP-4, 035 was obtained in 55% yield using sulfonyl | >95% |
| | chloride I-014 (50 mg, 0.12 mmol, 1 equiv.), 1-Methylcyclohexan- | |
| | 1-amine hydrochloride (25 mg, 0.17 mmol, 1.5 equiv.) and iPr₂NEt | |
| | (58 μL, 0.34 mmol, 3 equiv.) in DCM (0.6 mL) at RT for 3 h. | |
| | Purification by PTLC (cHex/EtOAc = 30/70). | |
| 036 | According to GP-4, 035 was obtained in 97% yield using sulfonyl | >95% |
| | chloride I-014 (50 mg, 0.12 mmol, 1 equiv.), 2- | |
| | azabicyclo[2.2.1]heptane (17 μL, 0.17 mmol, 1.5 equiv.) and | |
| | iPr₂NEt (40 μL, 0.23 mmol, 2 equiv.) in DCM (0.6 mL) at RT for 3 h. | |
| | Purification by PTLC (DCM/MeOH = 98/2). | |
| 037 | To a solution of I-015 (60 mg, 0.11 mmol, 1 equiv.) in DMF (1 mL) | >95% |
| | at RT, were added K₂CO₃ (46 mg, 0.33 mmol, 3 equiv.) and MeI | |
| | (21 μL, 0.33 mmol, 3 equiv.) and the suspension was stirred at 50 °C | |
| | for 16 h. The reaction mixture was partitioned between EtOAc and | |
| | H₂O. The layers were separated and the aqueous phase was | |
| | extracted with EtOAc. The combined organic extracts were washed | |
| | with brine, dried (Na₂SO₄), filtered and concentrated under reduced | |
| | pressure. The residue was purified by PTLC (cHex/EtOAc = 30/70) | |
| | affording 037 in 88% yield. | |
| 038 | To a solution of I-016 (80 mg, 0.16 mmol, 1 equiv.) in DMF (0.8 mL) | >95% |
| | at RT, was added NaH (60% in mineral oil, 13 mg, 0.31 mmol, 2 | |
| | equiv.). After 15 min at RT, benzyl bromide (37 μL, 0.31 mmol, 2 | |
| | equiv.) was added and the solution was stirred at RT for 2 h. The | |
| | reaction mixture was partitioned between EtOAc and H₂O. The | |
| | layers were separated and the aqueous phase was extracted with | |
| | EtOAc. The combined organic extracts were washed with brine, | |
| | dried (Na₂SO₄), filtered and concentrated under reduced pressure. | |
| | The residue was purified by semi-preparative HPLC affording 038 | |
| | in 60% yield. | |
| 039 | To a solution of I-017 (101 mg, 0.20 mmol, 1 equiv.) in DMF (1 mL) | >95% |
| | at RT, was added NaH (60% in mineral oil, 13 mg, 0.30 mmol, 1.5 | |
| | equiv.). After 15 min at RT, benzyl bromide (48 μL, 0.41 mmol, 2 | |
| | equiv.) was added and the solution was stirred at RT for 2 h. The | |
| | reaction mixture was partitioned between EtOAc and H₂O. The | |
| | layers were separated and the aqueous phase was extracted with | |
| | EtOAc. The combined organic extracts were washed with brine, | |
| | dried (Na₂SO₄), filtered and concentrated under reduced pressure. | |
| | The residue was purified by semi-preparative HPLC affording 039 | |
| | in 53% yield. | |
| 040 | To a solution of I-018 (90 mg, 0.17 mmol, 1 equiv.) in DCE (1 mL) | >95% |
| | at RT, were added benzaldehyde (21 μL, 0.21 mmol, 1.2 equiv.) and | |
| | NaBH(OAc)₃ (73 mg, 0.34 mmol, 2 equiv.). The suspension was | |
| | stirred at RT for 16 h. The reaction mixture was partitioned between | |
| | EtOAc and aq. sat. NaHCO₃. The layers were separated and the | |
| | aqueous phase was extracted with EtOAc. The combined organic | |
| | extracts were washed with brine, dried (Na₂SO₄), filtered and | |
| | concentrated under reduced pressure. The residue was purified by | |
| | semi-preparative HPLC affording 040 in 63% yield. | |
| 041 | According to GP-5, 041 was obtained in 80% yield using 3-bromo- | >95% |
| | N,N-diethyl-benzenesulfonamide (150 mg, 513 μmol, 1 equiv.), [4- | |
| | fluoro-2-(trifluoromethyl)phenyl]-piperazin-1-yl-methanone | |
| | (156 mg, 565 μmol, 1.1 equiv.), Cs₂CO₃ (502 mg, 1.54 mmol, 3 | |
| | equiv.), Pd(OAc)₂ (12 mg, 51 μmol, 0.1 equiv.) and rac-BINAP | |
| | (38 mg, 62 μmol, 0.12 equiv.) in Toluene (2.6 mL) at reflux for 16 h. | |
| | Purification by FC (cHex/EtOAc = 95/5 to 30/70). | |
| 042 | According to GP-5, 042 was obtained in 81% yield using aryl | 95% |
| | bromide I-019 (104 mg, 335 μmol, 1 equiv.), [4-fluoro-2- | |
| | (trifluoromethyl)phenyl]-piperazin-1-yl-methanone (141 mg, | |
| | 510 μmol, 1.5 equiv.), Cs₂CO₃ (315 mg, 967 μmol, 2.9 equiv.), | |
| | Pd(OAc)₂ (10 mg, 44 μmol, 0.13 equiv.) and rac-BINAP (30 mg, | |
| | 48 μmol, 0.14 equiv.) in Toluene (1.6 mL) at reflux for 2.5 h. | |
| | Purification by FC (cHex/EtOAc = 100/0 to 70/30). | |
| 043 | According to GP-4, 043 was obtained in 37% yield using sulfonyl | >95% |
| | chloride I-021 (140 mg, 0.28 mmol, 1 equiv.), methyl-N- | |
| | propylamine (29 μL, 0.28 mmol, 1 equiv.) and Et₃N (59 μL, | |
| | 0.43 mmol, 1.5 equiv.) in DCM (1.5 mL) at RT for 2 h. Purification | |
| | by PTLC (cHex/EtOAc = 80/20). | |
| 044 | According to GP-6, 044 was obtained in 12% yield using aryl | >95% |
| | bromide I-022 (107 mg, 326 μmol, 1.2 equiv.), [4-fluoro-2- | |
| | (trifluoromethyl)phenyl]-piperazin-1-yl-methanone (75.0 mg, | |
| | 271 μmol, 1 equiv.), t-BuONa (39 mg, 0.41 mmol, 1.5 equiv.), | |
| | XantPhos-Pd-G3 (23 mg, 14 μmol, 0.05 equiv.) in Toluene (1.3 mL) | |
| | at reflux for 16 h. Purification by PTLC (cHex/EtOAc = 70/30). | |
| 045 | According to GP-6, 045 was obtained in 84% yield using aryl | >95% |
| | bromide I-023 (138 mg, 426 μmol, 1.2 equiv.), [4-fluoro-2- | |
| | (trifluoromethyl)phenyl]-piperazin-1-yl-methanone (98.0 mg, | |
| | 355 μmol, 1 equiv.), t-BuONa (51 mg, 0.53 mmol, 1.5 equiv.), | |
| | XantPhos-Pd-G3 (17 mg, 18 μmol, 0.05 equiv.) in Toluene (1.8 mL) | |
| | at reflux for 4 h. Purification by FC (cHex/EtOAc = 97/3 to 30/70). | |
| 046 | According to GP-5, 046 was obtained in 79% yield using aryl | >95% |
| | bromide I-025 (192 mg, 498 μmol, 1 equiv.), [4-fluoro-2- | |
| | (trifluoromethyl)phenyl]-piperazin-1-yl-methanone (206 mg, | |
| | 747 μmol, 1.5 equiv.), Cs₂CO₃ (324 mg, 996 μmol, 2 equiv.), | |
| | Pd(OAc)₂ (11 mg, 50 μmol, 0.1 equiv.) and rac-BINAP (47 mg, | |
| | 75 μmol, 0.15 equiv.) in Toluene (2.6 mL) at reflux for 3 h. | |
| | Purification by FC (cHex/EtOAc = 90/10 to 0/100). | |
| 047 | According to GP-5, 047 was obtained in 83% yield using aryl | >95% |
| | bromide I-026 (330 mg, 828 μmol, 1 equiv.), [4-fluoro-2- | |
| | (trifluoromethyl)phenyl]-piperazin-1-yl-methanone (434 mg, | |
| | 1.24 mol, 1.5 equiv.), Cs₂CO₃ (540 mg, 1.66 mmol, 2 equiv.), | |
| | Pd(OAc)₂ (19 mg, 83 μmol, 0.1 equiv.) and rac-BINAP (62 mg, | |
| | 0.10 mmol, 0.12 equiv.) in Toluene (4 mL) at reflux for 16 h. | |
| | Purification by FC (cHex/EtOAc = 100/0 to 20/80). | |
| 048 | A suspension of 047 (370 mg, 623 μmol, 1 equiv.) and Pd(OH)₂ | >95% |
| | (20% on activated charcoal, 50% wet, 41 mg, 33 μmol, 0.05 equiv.) | |
| | in EtOH (12 mL) was stirred under and H₂ atmosphere at RT for 5 h. | |
| | The suspension was filtered over celite (EtOH rinses), concentrated | |
| | and the residue was purified by FC (cHex/EtOAc = 100/0 to 0/100) | |
| | affording 048 in 96% yield. | |
| 049 | To a solution of 048 (70 mg, 0.14 mmol, 1 equiv.) in DMF (1.4 mL) | >95% |
| | at RT, were added K₂CO₃ (80 mg, 0.56 mmol, 4 equiv.) and 2- | |
| | bromoethanol (40 μL, 0.56 mmol, 4 equiv.) and the suspension was | |
| | stirred at 50 °C for 16 h. The reaction mixture was partitioned | |
| | between EtOAc and H₂O. The layers were separated and the | |
| | aqueous phase was extracted with EtOAc. The combined organic | |
| | extracts were washed with brine, dried (Na₂SO₄), filtered and | |
| | concentrated under reduced pressure. The residue was purified by | |
| | PTLC (DCM/MeOH = 95/5) affording 049 in 53% yield. | |
| 050 | According to GP-4, 050 was obtained in 64% yield using sulfonyl | >95% |
| | chloride I-014 (70 mg, 0.16 mmol, 1 equiv.), 4-(4- | |
| | fluorophenyl)piperidine (34 mg, 0.19 mmol, 1.2 equiv.) and Et₃N | |
| | (40 μL, 0.31 mmol, 2 equiv.) in DCM (0.8 mL) at RT for 16 h. | |
| | Purification by PTLC (cHex/EtOAc = 50/50). | |
| 051 | According to GP-4, 051 was obtained in 51% yield using sulfonyl | >95% |
| | chloride I-014 (70 mg, 0.16 mmol, 1 equiv.), 1-[2-(4- | |
| | chlorophenyl)ethyl]piperazine dihydrochloride (56 mg, 0.19 mmol, | |
| | 1.2 equiv.) and Et₃N (110 μL, 0.78 mmol, 5 equiv.) in DCM (0.8 mL) | |
| | at RT for 2 h. Purification by PTLC (cHex/EtOAc/EtOH = | |
| | 50/37/13). | |
| 052 | According to GP-4, 052 was obtained in 69% yield using sulfonyl | >95% |
| | chloride I-014 (70 mg, 0.16 mmol, 1 equiv.), 1-[2-(4- | |
| | fluorophenyl)ethyl]piperazine (39 mg, 0.19 mmol, 1.2 equiv.) and | |
| | Et₃N (40 μL, 0.31 mmol, 2 equiv.) in DCM (0.8 mL) at RT for 2 h. | |
| | Purification by PTLC (cHex/EtOAc/EtOH = 50/37/13). | |
| 053 | According to GP-4, 053 was obtained in 59% yield using sulfonyl | >95% |
| | chloride I-014 (70 mg, 0.16 mmol, 1 equiv.), 1-(2- | |
| | phenylpropyl)piperazine dihydrochloride (52 mg, 0.19 mmol, 1.2 | |
| | equiv.) and Et₃N (110 μL, 0.78 mmol, 5 equiv.) in DCM (0.8 mL) at | |
| | RT for 2 h. Purification by PTLC (cHex/EtOAc/EtOH = 50/37/13). | |
| 054 | According to GP-4, 054 was obtained in 85% yield using sulfonyl | >95% |
| | chloride I-014 (50 mg, 0.11 mmol, 1 equiv.), 1-[(2R*)-2-(4- | |
| | fluorophenyl)propyl]piperazine (27 mg, 0.12 mmol, 1.1 equiv.) and | |
| | iPr₂NEt (39 μL, 0.22 mmol, 2 equiv.) in DCM (1 mL) at RT for 16 h. | |
| | Purification by PTLC (cHex/EtOAc = 40/60). | |
| 055 | According to GP-4, 055 was obtained in 24% yield using sulfonyl | >95% |
| | chloride I-030 (168 mg, 0.375 mmol, 1 equiv.), 4-phenylpiperidine | |
| | (121 mg, 0.750 mmol, 2 equiv.) and Et₃N (100 μL, 0.750 mmol, 2 | |
| | equiv.) in DCM (2 mL) at RT for 2 h. Purification by FC | |
| | (cHex/EtOAc = 95/5 to 50/50). | |
| 056 | According to GP-4, 056 was obtained in 23% yield using sulfonyl | >95% |
| | chloride I-030 (168 mg, 0.375 mmol, 1 equiv.), 4-(4- | |
| | fluorophenyl)piperidine (134 mg, 0.750 mmol, 2 equiv.) and Et₃N | |
| | (100 μL, 0.750 mmol, 2 equiv.) in DCM (2 mL) at RT for 2 h. | |
| | Purification by FC (cHex/EtOAc = 95/5 to 50/50). | |
| 057 | According to GP-4, 057 was obtained in 58% yield using sulfonyl | >95% |
| | chloride I-034 (166 mg, 0.355 mmol, 1 equiv.), 4-fluoropiperidine | |
| | hydrochloride (74 mg, 0.53 mmol, 1.5 equiv.) and Et₃N (148 μL, | |
| | 1.06 mmol, 3 equiv.) in DCM (2 mL) at RT for 16 h. Purification by | |
| | FC (cHex/EtOAc = 95/5 to 50/50). | |
| 058 | According to GP-4, 058 was obtained in 10% yield using sulfonyl | >95% |
| | chloride I-034 (166 mg, 0.355 mmol, 1 equiv.), tert-butylamine | |
| | (56 μL, 0.53 mmol, 1.5 equiv.) and Et₃N (74 μL, 0.53 mmol, 1.5 | |
| | equiv.) in DCM (2 mL) at RT for 16 h. Purification by FC | |
| | (cHex/EtOAc = 95/5 to 50/50). | |
| 059 | According to GP-4, 059 was obtained in 49% yield using sulfonyl | >95% |
| | chloride I-034 (166 mg, 0.355 mmol, 1 equiv.), N- | |
| | (cyclopropylmethyl)-N-methylamine (45 mg, 0.53 mmol, 1.5 | |
| | equiv.) and Et₃N (74 μL, 0.53 mmol, 1.5 equiv.) in DCM (2 mL) at | |
| | RT for 16 h. Purification by FC (cHex/EtOAc = 95/5 to 50/50). | |
| 060 | According to GP-4, 060 was obtained in 82% yield using sulfonyl | >95% |
| | chloride I-014 (100 mg, 0.224 mmol, 1 equiv.), 2,2- | |
| | dimethylpyrrolidine (33 mg, 0.34 mmol, 1.5 equiv.) and iPr₂NEt | |
| | (50 μL, 0.27 mmol, 1.2 equiv.) in DCM (.1 mL) at RT for 1 h. | |
| | Purification by FC (cHex/EtOAc = 95/5 to 50/50). | |

### Analytical Data for the Final Compounds

The analytical data for the final compounds are presented on the following Table 3.

TABLE 3

| Cpd | Description |
|---|---|
| 001 | White solid. ¹H NMR (400 MHz, Chloroform-d) δ 7.55 (dd, J = 8.6, 2.0 Hz, |
| | 1H), 7.41 (s, 1H), 7.35 (dd, J = 8.5, 5.8 Hz, 1H), 7.21 (dd, J = 8.5, 2.4 Hz, 1H), |
| | 7.10 (td, J = 8.2, 2.4 Hz, 1H), 6.96 (d, J = 8.6 Hz, 1H), 4.05 (br s, 2H), 3.96 (s, |
| | 3H), 3.56-3.44 (m, 2H), 3.30-3.22 (m, 5H), 3.22-3.12 (m, 2H), 3.08-2.98 |
| | (m, 1H), 1.15 (t, J = 7.1 Hz, 6H). ¹⁹F NMR (376 MHz, Chloroform-d) δ −109.1. |
| | MS (ESI⁺): [M + H]⁺ 484.2/486.2. |
| 002 | White solid. ¹H NMR (400 MHz, Chloroform-d) δ 7.53 (dd, J = 8.5, 2.2 Hz, |
| | 1H), 7.35-7.29 (m, 2H), 7.22 (dd, J = 9.5, 8.0 Hz, 1H), 6.95 (d, J = 8.6 Hz, |
| | 1H), 4.00 (q, J = 5.3 Hz, 2H), 3.96 (s, 3H), 3.54-3.39 (m, 2H), 3.24 (q, J = 7.1 |
| | Hz, 4H), 3.20-2.96 (m, 4H), 1.15 (t, J = 7.1 Hz, 6H). ¹⁹F NMR (376 MHz, |
| | Chloroform-d) δ -132.70 (d, JF-F = 21.0 Hz), −137.12 (d, J_{F-F} = 21.1 Hz). MS |
| | (ESI⁺): [M + H]⁺ 502.2/504.2. |
| 003 | White solid. ¹H NMR (400 MHz, Chloroform-d) δ 7.52 (dd, J = 8.5, 2.1 Hz, |
| | 1H), 7.45 (d, J = 1.9 Hz, 1H), 7.36-7.26 (m, 3H), 6.96 (d, J = 8.5 Hz, 1H), 3.99 |
| | (t, J = 5.1 Hz, 2H), 3.94 (s, 3H), 3.52-3.33 (m, 2H), 3.29-3.06 (m, 7H), 3.04- |
| | 2.93 (m, 1H), 1.83-1.68 (m, 4H). MS (ESI⁺): [M + H]⁺ 498.1/500.1. |
| 004 | White solid. ¹H NMR (400 MHz, Chloroform-d) δ 7.55 (dd, J = 8.5, 2.1 Hz, |
| | 1H), 7.39-7.31 (m, 2H), 7.21 (dd, J = 8.5, 2.4 Hz, 1H), 7.10 (td, J = 8.3, 2.5 |
| | Hz, 1H), 6.98 (d, J = 8.5 Hz, 1H), 4.02 (t, J = 5.2 Hz, 2H), 3.97 (s, 3H), 3.51 |
| | (ddd, J = 13.2, 6.9, 3.2 Hz, 1H), 3.41 (ddd, J = 13.0, 6.5, 3.2 Hz, 1H), 3.30- |
| | 3.08 (m, 7H), 3.61-2.95 (m, 1H), 1.84-1.72 (m, 4H). ¹⁹F NMR (376 MHz, |
| | Chloroform-d) δ −109.2. MS (ESI⁺): [M + H]⁺ 482.1/484.1. |
| 005 | White solid. ¹H NMR (400 MHz, Chloroform-d) δ 7.51 (dd, J = 8.5, 2.2 Hz, |
| | 1H), 7.33 (s, 1H), 7.22 (dd, J = 9.5, 8.0 Hz, 1H), 6.98 (d, J = 8.6 Hz, 1H), 4.00 |
| | (q, J = 5.7 Hz, 2H), 3.96 (s, 3H), 3.56-3.38 (m, 2H), 3.25-3.08 (m, 3H), 3.07- |
| | 2.93 (m, 3H), 2.73 (s, 3H), 1.60-1.52 (m, 3H), 0.94 (t, J = 7.4 Hz, 3H). ¹⁹F |
| | NMR (376 MHz, Chloroform-d) δ −132.67 (d, J_{F-F} = 21.0 Hz), −137.10 (d, |
| | J_{F-F} = 21.1 Hz). MS (ESI⁺): [M + H]⁺ 502.2/504.2. |
| 006 | White solid. ¹H NMR (400 MHz, Chloroform-d) δ 7.48 (dd, J = 8.6, 2.1 Hz, |
| | 1H), 7.42 (dd, J = 8.9, 2.5 Hz, 1H), 7.33 (dd, J = 8.5, 5.3 Hz, 1H), 7.25-7.18 |
| | (m, 2H), 6.94 (d, 1H), 6.93 (t, JH-F = 52.8 Hz, 1H), 4.15-3.96 (m, 2H), 3.93 (s, |
| | 3H), 3.46 (d, J = 5.2 Hz, 2H), 3.15 (s, 2H), 3.07-2.90 (m, 4H), 2.70 (s, 3H), |
| | 1.61-1.49 (m, 2H), 0.92 (t, J = 7.4 Hz, 3H). ¹⁹F NMR (376 MHz, Chloroform- |
| | d) δ −106.19 (d, J = 305.4 Hz), −109.10, −122.47 (d, J = 300.1 Hz). MS (ESI⁺): |
| | [M + H]⁺ 500.5. |
| 007 | Yellow solid. ¹H NMR (400 MHz, Chloroform-d) δ 7.73 (dd, J = 5.3, 3.7 Hz, |
| | 1H), 7.58-7.50 (m, 2H), 7.45 (dd, J = 8.5, 2.1 Hz, 1H), 7.39-7.31 (m, 1H), |
| | 7.28 (d, J = 4.3 Hz, 2H), 7.24-7.17 (m, 2H), 7.13-6.79 (m, 4H), 4.14-3.88 |
| | (m, 5H), 3.83-3.69 (m, 2H), 3.47 (t, J = 5.0 Hz, 2H), 3.17 (t, J = 5.1 Hz, 2H), |
| | 3.00 (s, 2H), 2.53 (d, J = 6.6 Hz, 2H), 2.20 (td, J = 11.7, 2.4 Hz, 2H), 1.71 (dd, |
| | J = 12.9, 3.0 Hz, 2H), 1.53-1.27 (m, 3H). ¹⁹F NMR (376 MHz, Chloroform-d) |
| | δ −105.26 (d, J_{F-F} = 299.9 Hz), −121.42 (d, J_{F-F} = 299.6 Hz). MS (ESI⁺): [M + H]⁺ |
| | 584.3. |
| 008 | Yellow oil. ¹H NMR (400 MHz, Chloroform-d) δ 8.43 (d, J = 2.4 Hz, 1H), 7.57 |
| | (dd, J = 7.9, 2.4 Hz, 1H), 7.43 (dd, J = 8.5, 2.1 Hz, 1H), 7.28-7.14 (m, 4H), |
| | 7.11-7.04 (m, 2H), 6.93 (d, J = 8.5 Hz, 1H), 4.00 (t, J = 5.0 Hz, 2H), 3.93 (s, |
| | 3H), 3.73 (d, J = 11.6 Hz, 2H), 3.40 (t, J = 4.9 Hz, 2H), 3.18 (t, J = 5.0 Hz, 2H), |
| | 3.05 (t, J = 4.8 Hz, 2H), 2.51 (d, J = 6.5 Hz, 2H), 2.17 (dd, J = 12.5, 10.1 Hz, |
| | 2H), 1.72-1.62 (m, 2H), 1.47-1.27 (m, 3H). ). ¹⁹F NMR (376 MHz, |
| | Chloroform-d) δ −122.61. MS (ESI⁺): [M + H]⁺ 587.1/589.1. |
| 009 | White solid. ¹H NMR (400 MHz, Chloroform-d) δ 7.46 (dd, J = 8.5, 2.2 Hz, |
| | 1H), 7.34-7.29 (m, 1H), 7.28-7.24 (m, 2H), 7.24-7.17 (m, 3H), 7.12-7.06 |
| | (m, 2H), 6.96 (d, J = 8.5 Hz, 1H), 4.04-3.97 (m, 2H), 3.96 (s, 3H), 3.76 (d, J = |
| | 11.5 Hz, 2H), 3.50-3.35 (m, 2H), 3.25-2.95 (m, 4H), 2.53 (d, J = 6.5 Hz, |
| | 2H), 2.19 (dd, J = 12.4, 10.1 Hz, 2H), 1.70 (d, J = 12.3 Hz, 2H), 1.48-1.31 (m, |
| | 3H). ¹⁹F NMR (376 MHz, Chloroform-d) δ −132.68 (d, J_{F-F} = 21.1 Hz), −137.10 |
| | (d, J_{F-F} = 21.0 Hz). MS (ESI⁺): [M + H]⁺ 604.2/606.2. |
| 010 | White solid. ¹H NMR (400 MHz, Chloroform-d) δ 7.42 (ddd, J = 9.0, 5.0, 2.3 |
| | Hz, 2H), 7.32 (dd, J = 8.5, 5.2 Hz, 1H), 7.28-7.26 (m, 1H), 7.24-7.16 (m, |
| | 3H), 7.12-7.02 (m, 2H), 6.93 (d, 1H), 6.92 (t, JH-F = 55.6 Hz, 1H), 4.16-3.93 |
| | (m, 2H), 3.93 (s, 3H), 3.73 (d, J = 11.5 Hz, 2H), 3.53-3.36 (m, 2H), 3.23- |
| | 3.06 (m, 2H), 2.98 (s, 2H), 2.51 (d, J = 6.6 Hz, 2H), 2.17 (dd, J = 12.7, 10.2 Hz, |
| | 2H), 1.75-1.62 (m, 2H), 1.48-1.26 (m, 4H). ¹⁹F NMR (376 MHz, Chloroform- |
| | d) δ −106.18 (d, J = 298.5 Hz), −109.11, −122.40 (d, J = 299.5 Hz). MS (ESI⁺): |
| | [M + H]⁺ 602.6. |
| 011 | White solid. ¹H NMR (400 MHz, DMSO-d₆) δ 7.58 (dd, J = 9.0, 2.4 Hz, 1H), |
| | 7.55-7.49 (m, 1H), 7.47-7.40 (m, 3H), 7.38-7.28 (m, 3H), 7.25-7.19 (m, |
| | 2H), 7.18-7.14 (m, 1H), 4.92 (s, 1H), 3.92 (s, 3H), 3.90-3.72 (m, 2H), 3.58- |
| | 3.49 (m, 2H), 3.31-3.28 (m, 2H), 3.16-3.08 (m, 2H), 3.04-2.96 (m, 2H), |
| | 2.61 (d, J = 9.2 Hz, 2H), 1.96 (td, J = 13.3, 4.5 Hz, 2H), 1.63 (d, J = 13.2 Hz, |
| | 2H). ¹⁹F NMR (376 MHz, DMSO-d₆) δ −110.09.MS (ESI⁺): [M + H]⁺ |
| | 588.2/590.2. |
| 012 | White solid. ¹H NMR (400 MHz, Chloroform-d) δ 7.47 (dd, J = 8.5, 2.0 Hz, |
| | 1H), 7.38-7.24 (m, 5H), 7.23-7.14 (m, 3H), 7.09 (td, J = 8.3, 2.5 Hz, 1H), |
| | 6.96 (d, J = 8.6 Hz, 1H), 4.01 (t, J = 5.1 Hz, 2H), 3.95 (s, 3H), 3.59 (d, J = 11.3 |
| | Hz, 2H), 3.50-3.37 (m, 2H), 3.26-3.07 (m, 3H), 3.04-2.95 (m, 1H), 2.75 (s, |
| | 2H), 2.64-2.54 (m, 2H), 1.82 (td, J = 13.1, 4.6 Hz, 2H), 1.59 (s, 2H), 1.04 (s, |
| | 1H). ¹⁹F NMR (376 MHz, Chloroform-d) δ −109.22. MS (ESI⁺): [M + H]⁺ |
| | 602.2/604.2. |
| 013 | White solid. ¹H NMR (400 MHz, DMSO-d₆) δ 7.57 (dd, J = 9.0, 2.5 Hz, 1H), |
| | 7.54-7.48 (m, 1H), 7.35 (dd, J = 8.0, 4.0 Hz, 2H), 7.30-7.25 (m, 2H), 7.25- |
| | 7.17 (m, 4H), 7.08 (d, J = 2.2 Hz, 1H), 3.90 (s, 3H), 3.88-3.71 (m, 2H), 3.45 |
| | (s, 2H), 3.31-3.26 (m, 2H), 3.14-3.07 (m, 2H), 3.01-2.94 (m, 2H), 2.85 (s, |
| | 4H), 2.45-2.37 (m, 4H). ¹⁹F NMR (376 MHz, DMSO-d₆) δ −110.09.MS (ESI⁺): |
| | [M + H]⁺ 587.0/589.0. |
| 014 | White solid. ¹H NMR (400 MHz, Chloroform-d) δ 7.50 (dd, J = 8.5, 2.2 Hz, |
| | 1H), 7.38-7.32 (m, 1H), 7.30 (d, J = 2.2 Hz, 1H), 7.21 (dd, J = 8.5, 2.4 Hz, |
| | 1H), 7.10 (td, J = 8.2, 2.5 Hz, 1H), 6.98 (d, J = 8.6 Hz, 1H), 4.03 (t, J = 5.1 Hz, |
| | 2H), 3.97 (s, 3H), 3.55-3.38 (m, 2H), 3.28-3.10 (m, 3H), 3.01 (d, J = 7.4 Hz, |
| | 3H), 2.69 (s, 3H), 2.53 (h, J = 7.7 Hz, 1H), 2.12-2.00 (m, 2H), 1.99-1.81 (m, |
| | 2H), 1.80-1.67 (m, 2H). ¹⁹F NMR (376 MHz, Chloroform-d) δ −109.2. MS |
| | (ESI⁺): [M + H]⁺ 510.1/512.1. |
| 015 | White solid. ¹H NMR (400 MHz, Chloroform-d) δ 7.51 (dd, J = 8.5, 2.1 Hz, |
| | 1H), 7.38-7.30 (m, 2H), 7.21 (dd, J = 8.5, 2.4 Hz, 1H), 7.10 (td, J = 8.3, 2.5 |
| | Hz, 1H), 6.98 (d, J = 8.6 Hz, 1H), 4.07-4.01 (m, 2H), 3.96 (s, 3H), 3.55-3.40 |
| | (m, 2H), 3.28-3.11 (m, 3H), 3.07-2.98 (m, 1H), 2.79 (d, J = 7.3 Hz, 2H), 2.72 |
| | (s, 3H), 1.81-1.49 (m, 6H), 1.31-1.14 (m, 3H), 0.94 (q, J = 11.3, 10.8 Hz, |
| | 2H). ¹⁹F NMR (376 MHz, Chloroform-d) δ −109.2. MS (ESI⁺): [M + H]⁺ |
| | 538.2/540.2. |
| 016 | White solid. ¹H NMR (400 MHz, Chloroform-d) δ 7.52 (dd, J = 8.5, 2.2 Hz, |
| | 1H), 7.35 (dd, J = 8.5, 5.9 Hz, 1H), 7.30 (d, J = 2.2 Hz, 1H), 7.21 (dd, J = 8.5, |
| | 2.4 Hz, 1H), 7.10 (td, J = 8.2, 2.4 Hz, 1H), 6.95 (d, J = 8.5 Hz, 1H), 4.34 (p, J = |
| | 8.2 Hz, 1H), 4.07-3.99 (m, 2H), 3.96 (s, 3H), 3.51-3.36 (m, 2H), 3.24-3.05 |
| | (m, 3H), 3.05-2.96 (m, 1H), 2.70 (s, 3H), 1.69-1.61 (m, 3H), 1.55-1.31 (m, |
| | 5H). ¹⁹F NMR (376 MHz, Chloroform-d) δ −109.21.MS (ESI⁺): [M + H]⁺ |
| | 510.2/512.2. |
| 017 | White solid. ¹H NMR (400 MHz, Chloroform-d) δ 7.53 (dd, J = 8.5, 2.2 Hz, |
| | 1H), 7.38-7.33 (m, 2H), 7.21 (dd, J = 8.5, 2.4 Hz, 1H), 7.10 (td, J = 8.2, 2.5 |
| | Hz, 1H), 6.96 (d, J = 8.6 Hz, 1H), 4.09-3.98 (m, 2H), 3.96 (s, 3H), 3.79-3.70 |
| | (m, 1H), 3.55-3.38 (m, 2H), 3.25-3.08 (m, 3H), 3.06-2.96 (m, 1H), 2.73 (s, |
| | 3H), 1.80-1.54 (m, 5H), 1.37-1.23 (m, 4H), 1.08-0.95 (m, 1H). ¹⁹F NMR |
| | (376 MHz, Chloroform-d) δ −109.2. MS (ESI⁺): [M + H]⁺ 524.2/526.1. |
| 018 | White solid. ¹H NMR (400 MHz, Chloroform-d) δ 7.56 (dd, J = 8.5, 2.3 Hz, |
| | 1H), 7.35-7.31 (m, 2H), 7.18 (dd, J = 8.5, 2.5 Hz, 1H), 7.08 (td, J = 8.3, 2.4 |
| | Hz, 1H), 6.95 (d, J = 8.6 Hz, 1H), 4.86 (d, J = 6.0 Hz, 2H), 4.29-4.26 (m, 2H), |
| | 4.02-3.97 (m, 2H), 3.95 (s, 3H), 3.52-3.36 (m, 2H), 3.25-3.07 (m, 3H), 3.03- |
| | 2.95 (m, 1H), 2.50 (s, 3H), 1.72 (s, 3H). ¹⁹F NMR (376 MHz, Chloroform-d) |
| | δ −109.2. MS (ESI⁺): [M + H]⁺ 512.1/514.1. |
| 019 | White solid. ¹H NMR (400 MHz, Chloroform-d) δ 7.57 (dd, J = 8.5, 2.2 Hz, |
| | 1H), 7.39-7.29 (m, 7H), 7.20 (dd, J = 8.5, 2.4 Hz, 1H), 7.10 (td, J = 8.2, 2.5 |
| | Hz, 1H), 7.02 (d, J = 8.5 Hz, 1H), 4.15 (s, 2H), 4.03 (t, J = 5.1 Hz, 2H), 3.99 (s, |
| | 3H), 3.55-3.39 (m, 2H), 3.20 (dtd, J = 25.4, 13.9, 12.9, 6.8 Hz, 3H), 3.06- |
| | 2.97 (m, 1H), 2.62 (s, 3H). ¹⁹F NMR (376 MHz, Chloroform-d) δ −109.2. MS |
| | (ESI⁺): [M + H]⁺ 532.1/534.1. |
| 020 | White solid. ¹H NMR (400 MHz, Chloroform-d) δ 7.56 (dd, J = 8.5, 2.2 Hz, |
| | 1H), 7.38-7.29 (m, 5H), 7.25-7.18 (m, 2H), 7.10 (td, J = 8.2, 2.5 Hz, 1H), |
| | 7.02 (d, J = 8.6 Hz, 1H), 4.14 (s, 2H), 4.03 (t, J = 5.1 Hz, 2H), 3.99 (s, 3H), 3.56- |
| | 3.39 (m, 2H), 3.20 (dtd, J = 25.7, 14.2, 13.0, 6.7 Hz, 3H), 3.06-2.98 (m, 1H), |
| | 2.64 (s, 3H). ¹⁹F NMR (376 MHz, Chloroform-d) δ −109.2. MS (ESI⁺): [M + H]⁺ |
| | 566.1/568.1. |
| 021 | White solid. ¹H NMR (400 MHz, Chloroform-d) δ 7.56 (dd, J = 8.5, 2.2 Hz, |
| | 1H), 7.38-7.29 (m, 4H), 7.20 (dd, J = 8.4, 2.4 Hz, 1H), 7.13-6.99 (m, 4H), |
| | 4.11 (s, 2H), 4.03 (t, J = 5.2 Hz, 2H), 3.99 (s, 3H), 3.56-3.39 (m, 2H), 3.20 |
| | (ddd, J = 24.8, 12.2, 6.6 Hz, 3H), 3.07-2.98 (m, 1H), 2.61 (s, 3H). ¹⁹F NMR |
| | (376 MHz, Chloroform-d) δ −109.15, −114.30. MS (ESI⁺): [M + H]⁺ 550.1/552.1. |
| 022 | White solid. ¹H NMR (400 MHz, DMSO-d₆) δ 7.58 (dd, J = 9.0, 2.5 Hz, 1H), |
| | 7.55-7.49 (m, 1H), 7.44 (dd, J = 8.5, 2.1 Hz, 1H), 7.34 (td, J = 8.5, 2.5 Hz, |
| | 1H), 7.22-7.13 (m, 2H), 3.89 (s, 3H), 3.87-3.70 (m, 2H), 3.31-3.22 (m, 5H), |
| | 3.10 (s, 2H), 3.02-2.65 (m, 9H), 1.34 (s, 9H). ¹⁹F NMR (376 MHz, DMSO-d₆) |
| | δ -110.07. MS (ESI⁺): [M + H]⁺ 623.3/625.3 |
| 023 | White solid. ¹H NMR (400 MHz, DMSO-d₆) δ 7.57 (dd, J = 9.1, 2.4 Hz, 1H), |
| | 7.54-7.47 (m, 1H), 7.42-7.35 (m, 1H), 7.35-7.27 (m, 3H), 7.24-7.12 (m, |
| | 4H), 7.10 (d, J = 2.2 Hz, 1H), 6.33 (s, 1H), 3.88 (s, 3H), 3.84-3.69 (m, 2H), |
| | 3.30-3.25 (m, 4H), 3.3.13-3.05 (m, 2H), 3.03-2.95 (m, 4H), 2.93-2.86 (m, |
| | 2H), 2.41-2.36 (m, 2H). ¹⁹F NMR (376 MHz, DMSO-d₆) δ -110.09. MS (ESI⁺): |
| | [M + H]⁺ 584.0/586.0. |
| 024 | White solid. ¹H NMR (400 MHz, Chloroform-d) δ 7.47 (dd, J = 8.5, 2.1 Hz, |
| | 1H), 7.35 (dd, J = 8.5, 5.9 Hz, 1H), 7.30 (s, 1H), 7.27-7.06 (m, 7H), 6.97 (d, |
| | J = 8.5 Hz, 1H), 4.01 (t, J = 5.2 Hz, 2H), 3.97 (s, 3H), 3.76 (d, J = 11.4 Hz, 2H), |
| | 3.50-3.36 (m, 2H), 3.26-2.96 (m, 4H), 2.65-2.57 (m, 2H), 2.23 (td, J = 11.8, |
| | 2.5 Hz, 2H), 1.82-1.74 (m, 2H), 1.64-1.58 (m, 2H), 1.36 (qd, J = 12.1, 4.0 |
| | Hz, 2H), 1.27-1.15 (m, 1H). ¹⁹F NMR (376 MHz, Chloroform-d) δ −109.20. |
| | MS (ESI⁺): [M + H]⁺ 600.2/602.2. |
| 025 | White solid. ¹H NMR (400 MHz, Chloroform-d) δ 7.52 (dd, J = 8.5, 2.1 Hz, |
| | 1H), 7.39-7.29 (m, 4H), 7.26-7.14 (m, 4H), 7.10 (td, J = 8.2, 2.4 Hz, 1H), |
| | 7.01 (d, J = 8.5 Hz, 1H), 4.07-4.00 (m, 2H), 3.98 (s, 3H), 3.94 (d, J = 11.6 Hz, |
| | 2H), 3.51-3.38 (m, 2H), 3.30-2.98 (m, 4H), 2.49-2.33 (m, 3H), 1.95-1.79 |
| | (m, 4H). ¹⁹F NMR (376 MHz, Chloroform-d) δ −109.20. MS (ESI⁺): [M + H]⁺ |
| | 572.2/574.2. |
| 026 | White solid. ¹H NMR (400 MHz, Chloroform-d) δ 7.46 (dd, J = 8.5, 2.1 Hz, |
| | 1H), 7.38-7.30 (m, 3H), 7.28-7.15 (m, 5H), 7.09 (td, J = 8.2, 2.5 Hz, 1H), |
| | 6.97 (d, J = 8.5 Hz, 1H), 4.01 (t, J = 4.7 Hz, 2H), 3.97 (s, 3H), 3.90-3.79 (m, |
| | 2H), 3.50-3.35 (m, 2H), 3.26-2.83 (m, 5H), 2.39-2.20 (m, 2H), 1.97 (d, J = |
| | 13.3 Hz, 1H), 1.93-1.73 (m, 2H), 1.45 (qd, J = 12.4, 4.1 Hz, 1H). ¹⁹F NMR |
| | (376 MHz, Chloroform-d) δ −109.20. MS (ESI⁺): [M + H]⁺ 572.2/574.2. |
| 027 | White solid. ¹H NMR (400 MHz, DMSO-d₆) δ 7.58 (dd, J = 8.9, 2.4 Hz, 1H), |
| | 7.55-7.48 (m, 1H), 7.39-7.29 (m, 2H), 7.26 (t, J = 7.3 Hz, 2H), 7.22-7.11 |
| | (m, 4H), 7.02 (d, J = 2.2 Hz, 1H), 3.89 (s, 3H), 3.85-3.72 (m, 2H), 3.41 (d, |
| | J = 11.3 Hz, 1H), 3.37-3.31 (m, 2H), 3.08 (s, 2H), 2.96 (s, 2H), 2.49-2.41 (m, |
| | 3H), 2.24 (t, J = 11.2 Hz, 1H), 2.01 (t, J = 10.7 Hz, 1H), 1.82-1.70 (m, 1H), |
| | 1.70-1.60 (m, 1H), 1.60-1.51 (m, 1H), 1.47-1.33 (m, 1H), 0.91 (q, J = 10.9, |
| | 9.9 Hz, 1H). ¹⁹F NMR (376 MHz, DMSO-d₆) δ −110.05. MS (ESI⁺): [M + H]⁺ |
| | 586.1/588.1. |
| 028 | White solid. ¹H NMR (400 MHz, Chloroform-d) δ 7.58 (dd, J = 8.5, 2.0 Hz, |
| | 1H), 7.39-7.29 (m, 3H), 7.28-7.18 (m, 3H), 7.15-7.05 (m, 3H), 7.00 (d, J = |
| | 8.5 Hz, 1H), 4.02 (t, J = 5.0 Hz, 2H), 3.98 (s, 3H), 3.73 (dd, J = 8.8, 6.8 Hz, 1H), |
| | 3.58-3.52 (m, 1H), 3.50-3.45 (m, 1H), 3.45-3.33 (m, 2H), 3.32-3.09 (m, |
| | 5H), 3.06-2.97 (m, 1H), 2.23 (dt, J = 9.4, 6.2 Hz, 1H), 1.96-1.82 (m, 1H). ¹⁹F |
| | NMR (376 MHz, Chloroform-d) δ −109.20. MS (ESI⁺): [M + H]⁺ 558.2/560.1. |
| 029 | White solid. ¹H NMR (400 MHz, DMSO-d₆) δ 7.58 (dd, J = 9.0, 2.5 Hz, 1H), |
| | 7.52 (dd, J = 8.5, 6.1 Hz, 1H), 7.43 (dd, J = 8.5, 2.1 Hz, 1H), 7.35 (td, J = 8.5, |
| | 2.5 Hz, 1H), 7.25 (t, J = 7.4 Hz, 2H), 7.17 (dd, J = 8.0, 2.4 Hz, 2H), 7.12 (d, J = |
| | 2.2 Hz, 1H), 7.10-7.03 (m, 2H), 3.89 (s, 3H), 3.87-3.71 (m, 2H), 3.31-3.17 |
| | (m, 4H), 3.13-3.01 (m, 3H), 2.99-2.89 (m, 2H), 2.80 (dd, J = 10.0, 7.4 Hz, |
| | 1H), 2.47 (d, J = 7.6 Hz, 2H), 2.21 (h, J = 7.3 Hz, 1H), 1.78 (dt, J = 12.6, 6.2 |
| | Hz, 1H), 1.41 (dq, J = 12.5, 8.0 Hz, 1H). ¹⁹F NMR (376 MHz, DMSO-d₆) |
| | δ −110.07.MS (ESI⁺): [M + H]⁺ 572.0/574.0. |
| 030 | White solid. ¹H NMR (400 MHz, Chloroform-d) δ 7.50 (dd, J = 8.5, 2.0 Hz, |
| | 1H), 7.35 (dd, J = 8.4, 5.8 Hz, 1H), 7.28 (s, 1H), 7.21 (dd, J = 8.4, 2.4 Hz, 1H), |
| | 7.10 (td, J = 8.2, 2.4 Hz, 1H), 6.97 (d, J = 8.5 Hz, 1H), 4.02 (t, J = 4.9 Hz, 2H), |
| | 3.97 (s, 3H), 3.51-3.37 (m, 2H), 3.27-3.08 (m, 3H), 2.99 (dt, J = 14.4, 7.4 |
| | Hz, 3H), 2.73 (s, 3H), 1.57 (h, J = 7.3 Hz, 2H), 0.94 (t, J = 7.4 Hz, 3H). ¹⁹F |
| | NMR (376 MHz, Chloroform-d) δ −109.19. MS (ESI⁺): [M + H]⁺ 484.1/486.1. |
| 031 | White solid. ¹H NMR (400 MHz, Chloroform-d) δ 7.46 (dd, J = 8.5, 2.2 Hz, |
| | 1H), 7.33 (dd, J = 8.5, 5.8 Hz, 1H), 7.23 (d, J = 2.2 Hz, 1H), 7.18 (dd, J = 8.4, |
| | 2.4 Hz, 1H), 7.07 (td, J = 8.2, 2.5 Hz, 1H), 6.96 (d, J = 8.6 Hz, 1H), 4.00 (t, J = |
| | 5.1 Hz, 2H), 3.95 (s, 3H), 3.87 (d, J = 12.0 Hz, 2H), 3.53-3.35 (m, 2H), 3.25- |
| | 3.05 (m, 3H), 3.03-2.95 (m, 1H), 2.28 (td, J = 12.2, 2.5 Hz, 2H), 1.94 (dd, |
| | J = 19.7, 8.8 Hz, 3H), 1.68 (qd, J = 12.5, 4.2 Hz, 2H). ¹⁹F NMR (376 MHz, |
| | Chloroform-d) δ −73.71, −109.14. MS (ESI⁺): [M + H]⁺ 564.0/566.0. |
| 032 | White solid. ¹H NMR (400 MHz, Chloroform-d) δ 7.58 (dd, J = 8.6, 2.2 Hz, |
| | 1H), 7.38 (d, J = 2.3 Hz, 1H), 7.35-7.31 (m, 1H), 7.18 (dd, J = 8.4, 2.4 Hz, |
| | 1H), 7.07 (td, J = 8.2, 2.4 Hz, 1H), 6.91 (d, J = 8.6 Hz, 1H), 4.54 (s, 1H), 3.98 |
| | (t, J = 5.1 Hz, 2H), 3.93 (s, 3H), 3.50-3.35 (m, 2H), 3.21-3.06 (m, 3H), 3.00- |
| | 2.94 (m, 1H), 1.21 (s, 9H). ¹⁹F NMR (376 MHz, Chloroform-d) δ −109.3. MS |
| | (ESI⁺): [M + H]⁺ 484.1/486.0. |
| 033 | White solid. ¹H NMR (400 MHz, Chloroform-d) δ 7.50 (dd, J = 8.6, 2.3 Hz, |
| | 1H), 7.34-7.30 (m, 2H), 7.18 (dd, J = 8.5, 2.5 Hz, 1H), 7.07 (td, J = 8.2, 2.4 |
| | Hz, 1H), 6.90 (d, J = 8.6 Hz, 1H), 3.99 (t, J = 5.1 Hz, 2H), 3.92 (s, 3H), 3.50 - |
| | 3.35 (m, 2H), 3.21-2.94 (m, 4H), 2.92 (s, 3H), 1.33 (s, 9H). ¹⁹F NMR (376 |
| | MHz, Chloroform-d) δ −109.3. MS (ESI⁺): [M + H]⁺ 498.1/500.1. |
| 034 | White solid. ¹H NMR (400 MHz, Chloroform-d) δ 7.57 (dd, J = 8.5, 2.2 Hz, |
| | 1H), 7.36-7.30 (m, 2H), 7.18 (dd, J = 8.5, 2.4 Hz, 1H), 7.07 (td, J = 8.2, 2.5 |
| | Hz, 1H), 6.93 (d, J = 8.6 Hz, 1H), 4.67 (d, J = 7.6 Hz, 1H), 4.05-3.96 (m, 2H), |
| | 3.94 (s, 3H), 3.52-2.94 (m, 8H), 1.82-1.61 (m, 13H). ¹⁹F NMR (376 MHz, |
| | Chloroform-d) δ −109.2. MS (ESI⁺): [M + H]⁺ 562.2/564.2. |
| 035 | White solid. ¹H NMR (400 MHz, Chloroform-d) δ 7.59 (dd, J = 8.5, 2.2 Hz, |
| | 1H), 7.38 (d, J = 2.3 Hz, 1H), 7.33 (dd, J = 8.5, 5.9 Hz, 1H), 7.18 (dd, J = 8.4, |
| | 2.4 Hz, 1H), 7.07 (td, J = 8.2, 2.4 Hz, 1H), 6.91 (d, J = 8.6 Hz, 1H), 4.38 (s, 1H), |
| | 3.99 (t, J = 5.2 Hz, 2H), 3.93 (s, 3H), 3.52-3.35 (m, 2H), 3.21-3.06 (m, 3H), |
| | 3.01-2.94 (m, 1H), 1.73-1.65 (m, 2H), 1.44-1.26 (m, 8H), 1.20 (s, 3H). ¹⁹F |
| | NMR (376 MHz, Chloroform-d) δ −109.3. MS (ESI⁺): [M + H]⁺ 524.1/526.1. |
| 036 | White solid. ¹H NMR (400 MHz, Chloroform-d) δ 7.51 (dd, J = 8.5, 2.2 Hz, |
| | 1H), 7.34-7.30 (m, 2H), 7.17 (dd, J = 8.4, 2.4 Hz, 1H), 7.07 (td, J = 8.2, 2.5 |
| | Hz, 1H), 6.94 (d, J = 8.5 Hz, 1H), 4.15 (s, 1H), 3.98 (t, J = 5.2 Hz, 2H), 3.93 (s, |
| | 3H), 3.50-3.35 (m, 2H), 3.22-2.94 (m, 6H), 2.46 (d, J = 3.4 Hz, 1H), 1.79 - |
| | 1.73 (m, 1H), 1.64-1.52 (m, 2H), 1.40-1.35 (m, 1H), 1.26-1.17 (m, 1H), |
| | 1.04-1.00 (m, 1H). ¹⁹F NMR (376 MHz, Chloroform-d) δ −109.2. MS (ESI⁺): |
| | [M + H]⁺ 508.1/510.1. |
| 037 | White solid. ¹H NMR (400 MHz, DMSO-d₆) δ 7.58 (dd, J = 9.0, 2.5 Hz, 1H), |
| | 7.51 (dd, J = 8.6, 6.1 Hz, 1H), 7.42 (dd, J = 8.5, 2.2 Hz, 1H), 7.34 (td, J = 8.5, |
| | 2.5 Hz, 1H), 7.18 (d, J = 8.6 Hz, 1H), 7.14 (d, J = 2.2 Hz, 1H), 3.89 (s, 3H), 3.85- |
| | 3.74 (m, 2H), 3.31-3.26 (m, 2H), 3.10 (t, J = 5.5 Hz, 2H), 2.98 (t, J = 6.8 Hz, |
| | 4H), 2.64 (s, 3H), 2.37-2.18 (m, 2H), 1.68 (p, J = 7.3 Hz, 2H). ¹⁹F NMR (376 |
| | MHz, DMSO-d₆) δ −64.66, −110.06. MS (ESI⁺): [M + H]⁺ 552.1/554.1. |
| 038 | White solid. ¹H NMR (400 MHz, Chloroform-d) δ 7.48 (dd, J = 8.5, 2.2 Hz, |
| | 1H), 7.38-7.23 (m, 7H), 7.20 (dd, J = 8.4, 2.5 Hz, 1H), 7.09 (td, J = 8.2, 2.5 |
| | Hz, 1H), 6.97 (d, J = 8.6 Hz, 1H), 4.49 (s, 2H), 4.01 (t, J = 5.2 Hz, 2H), 3.98 (s, |
| | 3H), 3.50-3.45 (m, 2H), 3.45-3.37 (m, 1H), 3.26-3.08 (m, 5H), 2.99 (dtd, J = 11.3, |
| | 7.9, 7.2, 3.1 Hz, 3H), 1.92 (ddd, J = 16.0, 8.2, 3.8 Hz, 2H), 1.80 (dtd, J = 13.7, |
| | 7.0, 3.6 Hz, 2H). ¹⁹F NMR (376 MHz, Chloroform-d) δ −109.21. MS |
| | (ESI⁺): [M + H]⁺ 602.1/604.1. |
| 039 | White solid. ¹H NMR (400 MHz, DMSO-d₆) δ 7.58 (dd, J = 9.0, 2.5 Hz, 1H), |
| | 7.51 (dd, J = 8.6, 6.1 Hz, 1H), 7.46 (dd, J = 8.5, 2.2 Hz, 1H), 7.34 (td, J = 8.5, |
| | 2.5 Hz, 1H), 7.30-7.21 (m, 3H), 7.18 (d, J = 2.2 Hz, 1H), 7.12 (d, J = 8.6 Hz, |
| | 1H), 7.09-7.04 (m, 2H), 4.35-4.22 (m, 2H), 4.09-3.98 (m, 1H), 3.85 (s, 3H), |
| | 3.81-3.65 (m, 2H), 3.30-3.19 (m, 5H), 3.13 (td, J = 9.5, 7.0 Hz, 1H), 3.05 (s, |
| | 2H), 2.93 (d, J = 5.5 Hz, 2H), 1.86 (dtd, J = 14.3, 9.3, 8.8, 5.7 Hz, 2H). ¹⁹F NMR |
| | (376 MHz, DMSO-d₆) δ −109.25. MS (ESI⁺): [M + H]⁺ 588.1/590.1. |
| 040 | White solid. ¹H NMR (400 MHz, Chloroform-d, multiple sets of rotamers) δ |
| | 8.24-8.18 (m, 0.5H), 7.49 (dd, J = 8.5, 2.2 Hz, 1H), 7.37-7.26 (m, 6H), 7.25- |
| | 7.24 (m, 0.5H), 7.18 (dd, J = 8.4, 2.4 Hz, 1H), 7.07 (td, J = 8.2, 2.5 Hz, 1H), |
| | 6.98 (d, J = 8.6 Hz, 1H), 3.99 (t, J = 5.1 Hz, 2H), 3.95 (s, 3H), 3.72 (s, 2H), 3.48- |
| | 3.34 (m, 2H), 3.24-2.91 (m, 10H), 2.83 (s, 2H), 2.39 (dd, J = 9.8, 4.8 Hz, |
| | 2H). ¹⁹F NMR (376 MHz, Chloroform-d) δ −109.17. MS (ESI⁺): [M + H]⁺ |
| | 613.2/615.1. |
| 041 | Yellow solid. ¹H NMR (400 MHz, Chloroform-d) δ 7.47 (dd, J = 8.8, 2.4 Hz, |
| | 1H), 7.44-7.29 (m, 5H), 7.07 (dd, J = 8.2, 2.6 Hz, 1H), 4.08-3.99 (m, 1H), |
| | 3.99-3.90 (m, 1H), 3.40-3.32 (m, 4H), 3.26 (q, J = 7.1 Hz, 4H), 3.19-3.12 |
| | (m, 2H), 1.16 (t, J = 7.1 Hz, 6H). ¹⁹F NMR (376 MHz, Chloroform-d) |
| | δ −59.7, −108.9. MS (ESI⁺): [M + H]⁺ 488.1. |
| 042 | Brown solid. ¹H NMR (400 MHz, Chloroform-d) δ 7.45 (dd, J = 8.8, 2.4 Hz, |
| | 1H), 7.43-7.28 (m, 4H), 7.16 (dd, J = 11.9, 8.4 Hz, 1H), 4.04-3.93 (m, 2H), |
| | 3.41-3.32 (m, 2H), 3.20 (t, J = 5.2 Hz, 2H), 3.05-3.01 (m, 2H), 3.02-2.91 |
| | (m, 2H), 2.73 (s, 3H), 1.64-1.44 (m, 2H), 0.93 (t, J = 7.4 Hz, 3H). ¹⁹F NMR |
| | (376 MHz, Chloroform-d) 8 −60.20, −108.94, −115.62. (ESI⁺): [M + H]⁺ 506.1. |
| 043 | White solid. ¹H NMR (400 MHz, Chloroform-d) δ 7.44 (dd, J = 8.8, 2.5 Hz, |
| | 1H), 7.38 (dd, J = 8.5, 5.3 Hz, 1H), 7.34 (dd, J = 7.9, 2.5 Hz, 1H), 7.30 (s, 1H), |
| | 7.07 (d, J = 1.7 Hz, 1H), 4.71 (t, J = 8.9 Hz, 2H), 3.99-3.93 (m, 2H), 3.34 (t, |
| | J = 5.1 Hz, 2H), 3.30-3.11 (m, 4H), 3.09-2.99 (m, 2H), 2.97-2.88 (m, 2H), |
| | 2.70 (s, 3H), 1.60-1.50 (m, 2H), 0.92 (t, J = 7.4 Hz, 3H). ¹⁹F NMR (376 MHz, |
| | Chloroform-d) δ −60.24, −109.13. MS (ESI⁺): [M + H]⁺ 530.1. |
| 044 | White solid. ¹H NMR (400 MHz, Chloroform-d) δ 8.48 (d, J = 2.2 Hz, 1H), 7.64 |
| | (d, J = 2.2 Hz, 1H), 7.48 (dd, J = 8.8, 2.4 Hz, 1H), 7.44-7.32 (m, 2H), 4.19 - |
| | 4.05 (m, 1H), 4.05-3.93 (m, 1H), 3.48-3.37 (m, 2H), 3.30 (q, J = 7.1 Hz, 4H), |
| | 3.22 (t, J = 5.1 Hz, 2H), 3.05 (t, J = 5.1 Hz, 2H), 1.20 (t, J = 7.1 Hz, 6H). ¹⁹F |
| | NMR (376 MHz, Chloroform-d) δ −60.2, −108.8. MS (ESI⁺): [M + H]⁺ |
| | 523.0/525.0. |
| 045 | White solid. ¹H NMR (400 MHz, Chloroform-d) δ 8.27 (d, J = 2.2 Hz, 1H), 7.46 |
| | (dd, J = 8.8, 2.5 Hz, 1H), 7.43-7.31 (m, 3H), 4.07 (s, 3H), 4.04-3.94 (m, 2H), |
| | 3.39 (t, J = 5.1 Hz, 2H), 3.26 (q, J = 7.1 Hz, 4H), 3.25-3.15 (m, 2H), 3.10 - |
| | 2.92 (m, 2H), 1.18 (t, J = 7.1 Hz, 6H). ¹⁹F NMR (376 MHz, Chloroform-d) |
| | δ −60.2, −109.0. MS (ESI⁺): [M + H]⁺ 519.1. |
| 046 | White solid. ¹H NMR (400 MHz, DMSO-d₆) δ 7.79 (dd, J = 9.0, 1.8 Hz, 1H), |
| | 7.70-7.60 (m, 2H), 7.37 (dd, J = 8.5, 2.2 Hz, 1H), 7.15 (d, J = 8.5 Hz, 1H), |
| | 7.11 (d, J = 2.2 Hz, 1H), 4.25-4.15 (m, 2H), 3.89-3.79 (m, 1H), 3.78-3.62 |
| | (m, 3H), 3.35-3.25 (m, 4H), 3.26-2.95 (m, 4H), 2.95-2.77 (m, 3H), 2.61 (s, |
| | 3H), 1.52-1.38 (m, 2H), 0.84 (t, J = 7.3 Hz, 3H). ¹⁹F NMR (376 MHz, DMSO- |
| | d₆) δ −58.9, −110.2. MS (ESI⁺): [M + H]⁺ 562.2. |
| 047 | White solid. ¹H NMR (400 MHz, DMSO-d₆) δ 7.77 (dd, J = 9.1, 2.1 Hz, 1H), |
| | 7.69-7.58 (m, 2H), 7.52-7.46 (m, 2H), 7.45-7.39 (m, 3H), 7.38-7.31 (m, |
| | 1H), 7.27 (d, J = 8.6 Hz, 1H), 7.15 (d, J = 2.3 Hz, 1H), 5.22 (s, 2H), 3.90-3.61 |
| | (m, 2H), 3.29-3.22 (m, 1H), 3.21-3.12 (m, 2H), 3.13-2.97 (m, 2H), 2.97- |
| | 2.79 (m, 3H), 2.61 (s, 3H), 1.46 (h, J = 7.3 Hz, 2H), 0.84 (t, J = 7.3 Hz, 3H). ¹⁹F |
| | NMR (376 MHz, DMSO-d₆) 8 −58.90, −110.19. MS (ESI⁺): [M + H]⁺ 594.2. |
| 048 | White solid. ¹H NMR (400 MHz, Chloroform-d) 8 7.58-7.52 (m, 2H), 7.45 |
| | (dd, J = 8.7, 2.5 Hz, 1H), 7.41 (dd, J = 8.6, 5.2 Hz, 1H), 7.35 (td, J = 8.1, 2.6 |
| | Hz, 1H), 7.15 (s, 1H), 7.08-7.06 (m, 1H), 4.16-4.01 (m, 1H), 3.96-3.85 (m, |
| | 1H), 3.40-3.36 (m, 2H), 3.07-2.90 (m, 4H), 2.85-2.75 (m, 2H), 2.71 (s, 3H), |
| | 1.60-1.51 (m, 2H), 0.92 (t, J = 7.4 Hz, 3H). ¹⁹F NMR (376 MHz, Chloroform- |
| | d) δ −60.21, −108.72. MS (ESI⁺): [M + H]⁺ 504.1. |
| 049 | White solid. ¹H NMR (400 MHz, Chloroform-d) δ 7.48 (dd, J = 8.5, 2.2 Hz, |
| | 1H), 7.44 (dd, J = 8.7, 2.5 Hz, 1H), 7.38 (dd, J = 8.6, 5.3 Hz, 1H), 7.33 (td, J = |
| | 8.1, 2.5 Hz, 1H), 7.29 (d, J = 2.2 Hz, 1H), 7.01 (d, J = 8.5 Hz, 1H), 4.28-4.17 |
| | (m, 2H), 4.01-3.96 (m, 2H), 3.95-3.89 (m, 2H), 3.36 (t, J = 5.0 Hz, 2H), 3.23- |
| | 3.13 (m, 2H), 3.01 (t, J = 5.0 Hz, 2H), 2.98-2.94 (m, 2H), 2.71 (s, 3H), 2.58 |
| | (s, 1H), 1.66-1.49 (m, 2H), 0.92 (t, J = 7.4 Hz, 3H). ¹⁹F NMR (376 MHz, |
| | Chloroform-d) δ −60.21, −109.06. MS (ESI⁺): [M + H]⁺ 548.0. |
| 050 | ¹H NMR (400 MHz, Chloroform-d) δ 7.52-7.45 (m, 1H), 7.36-7.30 (m, 1H), |
| | 7.26-7.23 (m, 1H), 7.21-7.14 (m, 1H), 7.12-7.03 (m, 3H), 7.02-6.93 (m, |
| | 3H), 4.04-3.97 (m, 2H), 3.96 (s, 3H), 3.94-3.86 (m, 2H), 3.53-3.45 (m, 1H), |
| | 3.44-3.34 (m, 1H), 3.25-3.05 (m, 3H), 3.05-2.93 (m, 1H), 2.47-2.29 (m, |
| | 3H), 1.94-1.71 (m, 4H). ¹⁹F NMR (376 MHz, Chloroform-d) |
| | δ −109.16, −116.42. MS (ESI⁺): [M + H]⁺ 590.2/592.1. |
| 051 | ¹H NMR (400 MHz, Chloroform-d) δ 7.48-7.42 (m, 1H), 7.35-7.29 (m, 1H), |
| | 7.25-7.15 (m, 4H), 7.10-7.03 (m, 3H), 6.95 (d, J = 8.6 Hz, 1H), 4.04-3.96 |
| | (m, 2H), 3.94 (s, 3H), 3.53-3.49 (m, 1H), 3.47-3.34 (m, 1H), 3.22-2.93 (m, |
| | 8H), 2.73-2.65 (m, 2H), 2.64-2.52 (m, 6H). ¹⁹F NMR (376 MHz, Chloroform- |
| | d) δ −109.2. MS (ESI⁺): [M + H]⁺ 635.3/637.2. |
| 052 | ¹H NMR (400 MHz, Chloroform-d) δ 7.48-7.42 (m, 1H), 7.37-7.28 (m, 1H), |
| | 7.24-7.15 (m, 2H), 7.14-7.03 (m, 3H), 6.99-6.90 (m, 3H), 4.07-3.96 (m, |
| | 2H), 3.94 (s, 3H), 3.47-3.43 (m, 1H), 3.43-3.33 (m, 1H), 3.25-2.93 (m, 8H), |
| | 2.75-2.65 (m, 2H), 2.64-2.52 (m, 6H). ¹⁹F NMR (376 MHz, Chloroform-d) |
| | δ −109.20, −117.05. MS (ESI⁺): [M + H]⁺ 619.3/621.2. |
| 053 | ¹H NMR (400 MHz, Chloroform-d) δ 7.46-7.40 (m, 1H), 7.36-7.29 (m, 1H), |
| | 7.29-7.26 (m, 1H), 7.26-7.23 (m, 1H), 7.21-7.12 (m, 5H), 7.10-7.03 (m, |
| | 1H), 6.98-6.91 (m, 1H), 4.03-3.96 (m, 2H), 3.94 (s, 3H), 3.52-3.33 (m, 2H), |
| | 3.27-3.04 (m, 3H), 3.04-2.91 (m, 5H), 2.91-2.81 (m, 1H), 2.57-2.34 (m, |
| | 6H), 1.19 (d, J = 6.9 Hz, 3H). ¹⁹F NMR (376 MHz, Chloroform-d) δ −109.22. |
| | MS (ESI⁺): [M + H]⁺ 615.3/617.2. |
| 054 | ¹H NMR (400 MHz, Chloroform-d) δ 7.43 (dd, J = 8.5, 2.2 Hz, 1H), 7.33 (dd, |
| | J = 8.5, 5.9 Hz, 1H), 7.19-7.17 (m, 2H), 7.13-7.02 (m, 3H), 6.94 (ddd, J = 8.7, |
| | 6.7, 1.8 Hz, 3H), 3.99 (t, J = 5.1 Hz, 2H), 3.94 (s, 3H), 3.52-3.35 (m, 2H), 3.22- |
| | 3.06 (m, 3H), 3.03-2.90 (m, 5H), 2.86 (q, J = 7.2 Hz, 1H), 2.55-2.34 (m, |
| | 6H), 1.18 (s, 1.5H), 1.16 (s, 1.5H). ¹⁹F NMR (376 MHz, Chloroform-d) |
| | δ −109.21, −117.13. MS (ESI⁺): [M + H]⁺ 633.2/635.3. |
| 055 | White solid. ¹H NMR (400 MHz, Chloroform-d) δ 7.40-7.26 (m, 4H), 7.23 |
| | (dd, J = 8.1, 6.7 Hz, 2H), 7.17-7.10 (m, 2H), 7.10-7.05 (m, 2H), 7.02 (td, |
| | J = 8.2, 2.5 Hz, 1H), 4.10-3.73 (m, 4H), 3.50-3.27 (m, 2H), 2.99-2.68 (m, |
| | 4H), 2.44-2.21 (m, 6H), 1.93-1.69 (m, 4H). ¹⁹F NMR (376 MHz, Chloroform- |
| | d) δ −108.76. MS (ESI⁺): [M + H]⁺ 572.2/574.2. |
| 056 | White solid. ¹H NMR (400 MHz, Chloroform-d) δ 7.50-7.40 (m, 3H), 7.38 |
| | (dd, J = 8.5, 5.8 Hz, 1H), 7.22 (dd, J = 8.4, 2.4 Hz, 1H), 7.16-7.07 (ddd, J = |
| | 9.0, 5.6, 2.8 Hz, 3H), 7.06-6.94 (m, 2H), 4.15-3.85 (m, 4H), 3.60-3.48 (m, |
| | 1H), 3.48-3.37 (m, 1H), 3.10-2.77 (m, 4H), 2.55-2.30 (m, 6H), 1.96-1.72 |
| | (m, 4H). ¹⁹F NMR (376 MHz, Chloroform-d) δ −108.74, −116.39. MS (ESI⁺): |
| | [M + H]⁺ 590.1/592.1. |
| 057 | White solid. ¹H NMR (400 MHz, Chloroform-d) δ 7.58 (d, J = 2.0 Hz, 1H), 7.39- |
| | 7.30 (m, 2H), 7.19 (dd, J = 8.4, 2.4 Hz, 1H), 7.09 (td, J = 8.2, 2.5 Hz, 1H), |
| | 4.90-4.80 (m, 0.5H), 4.80-4.68 (m, 0.5H), 4.02 (t, J = 5.1 Hz, 2H), 3.56 - |
| | 3.45 (m, 1H), 3.45-3.30 (m, 3H), 3.17-2.93 (m, 3H), 2.93-2.77 (m, 3H), |
| | 2.10-1.82 (m, 4H). ¹⁹F NMR (376 MHz, Chloroform-d) δ −108.68, −186.28. |
| | MS (ESI⁺): [M + H]⁺ 534.0/536.0/538.0 |
| 058 | White solid. ¹H NMR (400 MHz, Chloroform-d) δ 7.72 (d, J = 2.0 Hz, 1H), 7.53 |
| | (d, J = 2.1 Hz, 1H), 7.34 (dd, J = 8.5, 5.8 Hz, 1H), 7.19 (dd, J = 8.4, 2.4 Hz, |
| | 1H), 7.09 (td, J = 8.2, 2.5 Hz, 1H), 4.43 (s, 1H), 4.01 (t, J = 5.1 Hz, 2H), 3.57 - |
| | 3.35 (m, 2H), 3.12-2.79 (m, 4H), 1.25 (s, 9H). ¹⁹F NMR (376 MHz, |
| | Chloroform-d) δ −108.73. MS (ESI⁺): [M + H]⁺ 504.0/506.0/5.08.0. |
| 059 | White solid. ¹H NMR (400 MHz, Chloroform-d) δ 7.59 (d, J = 2.0 Hz, 1H), 7.39 |
| | (d, J = 2.1 Hz, 1H), 7.34 (dd, J = 8.5, 5.8 Hz, 1H), 7.19 (dd, J = 8.4, 2.4 Hz, |
| | 1H), 7.08 (td, J = 8.2, 2.5 Hz, 1H), 4.01 (t, 2H), 3.58-3.29 (m, 2H), 3.14-2.85 |
| | (m, 6H), 2.83 (s, 3H), 0.97-0.82 (m, 1H), 0.65-0.47 (m, 2H), 0.29-0.11 (m, |
| | 2H). ¹⁹F NMR (376 MHz, Chloroform-d) δ −108.70. MS (ESI⁺): [M + H]⁺ |
| | 516.0/518.0/520.0. |
| 060 | White solid. ¹H NMR (400 MHz, Chloroform-d) δ 7.47 (dd, J = 8.5, 2.2 Hz, |
| | 1H), 7.30 (d, J = 2.2 Hz, 1H), 7.26 (dd, J = 8.5, 5.9 Hz, 1H), 7.11 (dd, J = 8.4, |
| | 2.5 Hz, 1H), 7.00 (td, J = 8.2, 2.4 Hz, 1H), 6.84 (d, J = 8.6 Hz, 1H), 3.93 (t, J = |
| | 5.1 Hz, 2H), 3.86 (s, 3H), 3.47-3.32 (m, 2H), 3.32-3.24 (m, 2H), 3.18-2.99 |
| | (m, 3H), 2.96-2.86 (m, 1H), 1.79-1.64 (m, 4H), 1.37 (s, 6H). ¹⁹F NMR (376 |
| | MHz, Chloroform-d) δ −109.25. MS (ESI⁺): [M + H]⁺ 510.2/512.2. |

### Example 2: Biological Activity of the Compounds

The purpose of this experiment was to evaluate the GFRα1-RET activity of the compounds 001-060 according to the invention.

### Materials and Methods

The compounds were tested for their activating activity of Elk1 signaling using the previously developed reporter-gene-based system in cells expressing GFRα1-RET (MG87 murine fibroblast stably transfected with PathDetect Elk-1, GFRα1, and RET) disclosed in Sidorova, Y. A. et al.: “Persephin signaling through GFRα1: The potential for the treatment of Parkinson's disease.” Molecular and Cellular Neuroscience, July 2010, Vol. 44, pp. 223-232. DOI: 10.1016/j.mcn.2010.03.009. For EC₅₀ determination, a dose-response test was performed using 6 concentrations of each tested compound. Dose-response curves were fitted using the sigmoidal dose-response (variable slope) analysis in GraphPad Prism program (Graph Pad Inc) and EC₅₀ of agonist/activator activity was calculated. Dose-response experiments were all performed in duplicate, two times independently.

### Results

The results are presented on Table 4 below (* means “10 μM<EC₅₀<50 μM”, ** means “5 μM<EC₅₀<10 μM”, *** means “1 μM<EC₅₀<5 μM”, and **** means “EC₅₀<1 μM”).

TABLE 4

| | | Luciferase |
|---|---|---|
| Cpd | Name | Assay EC₅₀ |
| 001 | 3-[4-(2-chloro-4-fluoro-benzoyl)piperazin-1-yl]-N,N- | ** |
| | diethyl-4-methoxy-benzenesulfonamide | |
| 002 | 3-[4-(2-chloro-4,5-difluoro-benzoyl)piperazin-1-yl]- | ** |
| | N,N-diethyl-4-methoxy-benzenesulfonamide | |
| 003 | (2,4-dichlorophenyl)-[4-(2-methoxy-5-pyrrolidin-1- | * |
| | ylsulfonyl-phenyl)piperazin-1-yl]methanone | |
| 004 | (2-chloro-4-fluoro-phenyl)-[4-(2-methoxy-5-pyrrolidin- | *** |
| | 1-ylsulfonyl-phenyl)piperazin-1-yl]methanone | |
| 005 | 3-[4-(2-chloro-4,5-difluoro-benzoyl)piperazin-1-yl]-4- | *** |
| | methoxy-N-methyl-N-propyl-benzenesulfonamide | |
| 006 | 3-[4-[2-(difluoromethyl)-4-fluoro-benzoyl]piperazin-1- | *** |
| | yl]-4-methoxy-N-methyl-N-propyl-benzenesulfonamide | |
| 007 | [4-[5-[(4-benzyl-1-piperidyl)sulfonyl]-2-methoxy- | *** |
| | phenyl]piperazin-1-yl]-[2- | |
| | (difluoromethyl)phenyl]methanone | |
| 008 | [4-[5-[(4-benzyl-1-piperidyl)sulfonyl]-2-methoxy- | ** |
| | phenyl]piperazin-1-yl]-(3-chloro-5-fluoro-2- | |
| | pyridyl)methanone | |
| 009 | [4-[5-[(4-benzyl-1-piperidyl)sulfonyl]-2-methoxy- | **** |
| | phenyl]piperazin-1-yl]-(2-chloro-4,5-difluoro- | |
| | phenyl)methanone | |
| 010 | [4-[5-[(4-benzyl-1-piperidyl)sulfonyl]-2-methoxy- | *** |
| | phenyl]piperazin-1-yl]-[2-(difluoromethyl)-4-fluoro- | |
| | phenyl]methanone | |
| 011 | (2-chloro-4-fluoro-phenyl)-[4-[5-[(4-hydroxy-4-phenyl- | ** |
| | 1-piperidyl)sulfonyl]-2-methoxy-phenyl]piperazin-1- | |
| | yl]methanone | |
| 012 | [4-[5-[(4-benzyl-4-hydroxy-1-piperidyl)sulfonyl]-2- | ** |
| | methoxy-phenyl]piperazin-1-yl]-(2-chloro-4-fluoro- | |
| | phenyl)methanone | |
| 013 | [4-[5-(4-benzylpiperazin-1-yl)sulfonyl-2-methoxy- | ** |
| | phenyl]piperazin-1-yl]-(2-chloro-4-fluoro- | |
| | phenyl)methanone | |
| 014 | 3-[4-(2-chloro-4-fluoro-benzoyl)piperazin-1-yl]-N- | ** |
| | (cyclobutylmethyl)-4-methoxy-N-methyl- | |
| | benzenesulfonamide | |
| 015 | 3-[4-(2-chloro-4-fluoro-benzoyl)piperazin-1-yl]-N- | ** |
| | (cyclohexylmethyl)-4-methoxy-N-methyl- | |
| | benzenesulfonamide | |
| 016 | 3-[4-(2-chloro-4-fluoro-benzoyl)piperazin-1-yl]-N- | ** |
| | cyclopentyl-4-methoxy-N-methyl-benzenesulfonamide | |
| 017 | 3-[4-(2-chloro-4-fluoro-benzoyl)piperazin-1-yl]-N- | *** |
| | cyclohexyl-4-methoxy-N-methyl-benzenesulfonamide | |
| 018 | 3-[4-(2-chloro-4-fluoro-benzoyl)piperazin-1-yl]-4- | * |
| | methoxy-N-methyl-N-(3-methyloxetan-3- | |
| | yl)benzenesulfonamide | |
| 019 | N-benzyl-3-[4-(2-chloro-4-fluoro-benzoyl)piperazin-1- | * |
| | yl]-4-methoxy-N-methyl-benzenesulfonamide | |
| 020 | 3-[4-(2-chloro-4-fluoro-benzoyl)piperazin-1-yl]-N-[(3- | *** |
| | chlorophenyl)methyl]-4-methoxy-N-methyl- | |
| | benzenesulfonamide | |
| 021 | 3-[4-(2-chloro-4-fluoro-benzoyl)piperazin-1-yl]-N-[(4- | ** |
| | fluorophenyl)methyl]-4-methoxy-N-methyl- | |
| | benzenesulfonamide | |
| 022 | tert-butyl 5-[3-[4-(2-chloro-4-fluoro-benzoyl)piperazin- | * |
| | 1-yl]-4-methoxy-phenyl]sulfonyl-1,3,3a,4,6,6a- | |
| | hexahydropyrrolo[3,4-c]pyrrole-2-carboxylate | |
| 023 | [4-[5-[(4-benzylidene-1-piperidyl)sulfonyl]-2-methoxy- | **** |
| | phenyl]piperazin-1-yl]-(2-chloro-4-fluoro- | |
| | phenyl)methanone | |
| 024 | (2-chloro-4-fluoro-phenyl)-[4-[2-methoxy-5-[[4-(2- | **** |
| | phenylethyl)-1-piperidyl]sulfonyl]phenyl]piperazin-1- | |
| | yl]methanone | |
| 025 | (2-chloro-4-fluoro-phenyl)-[4-[2-methoxy-5-[(4-phenyl- | **** |
| | 1-piperidyl)sulfonyl]phenyl]piperazin-1-yl]methanone | |
| 026 | (2-chloro-4-fluoro-phenyl)-[4-[2-methoxy-5-[[3-phenyl- | *** |
| | 1-piperidyl]sulfonyl]phenyl]piperazin-1-yl]methanone | |
| 027 | (2-chloro-4-fluoro-phenyl)-[4-[2-methoxy-5-[[3-benzyl- | *** |
| | 1-piperidyl]sulfonyl]phenyl]piperazin-1-yl]methanone | |
| 028 | (2-chloro-4-fluoro-phenyl)-[4-[2-methoxy-5-[3- | *** |
| | phenylpyrrolidin-1-yl]sulfonyl-phenyl]piperazin-1- | |
| | yl]methanone | |
| 029 | (2-chloro-4-fluoro-phenyl)-[4-[2-methoxy-5-[3- | ** |
| | benzylpyrrolidin-1-yl]sulfonyl-phenyl]piperazin-1- | |
| | yl]methanone | |
| 030 | 3-[4-(2-chloro-4-fluoro-benzoyl)piperazin-1-yl]-4- | *** |
| | methoxy-N-methyl-N-propyl-benzenesulfonamide | |
| 031 | (2-chloro-4-fluoro-phenyl)-[4-[2-methoxy-5-[[4- | *** |
| | (trifluoromethyl)-1-piperidyl]sulfonyl]phenyl]piperazin- | |
| | 1-yl]methanone | |
| 032 | N-tert-butyl-3-[4-(2-chloro-4-fluoro-benzoyl)piperazin- | *** |
| | 1-yl]-4-methoxy-benzenesulfonamide | |
| 033 | N-tert-butyl-3-[4-(2-chloro-4-fluoro-benzoyl)piperazin- | ** |
| | 1-yl]-4-methoxy-N-methyl-benzenesulfonamide | |
| 034 | N-(2-adamantyl)-3-[4-(2-chloro-4-fluoro- | ** |
| | benzoyl)piperazin-1-yl]-4-methoxy-benzenesulfonamide | |
| 035 | 3-[4-(2-chloro-4-fluoro-benzoyl)piperazin-1-yl]-4- | *** |
| | methoxy-N-(1-methylcyclohexyl)benzenesulfonamide | |
| 036 | (2-chloro-4-fluoro-phenyl)-[4-[2-methoxy-5-[[2- | * |
| | azabicyclo[2.2.1]heptan-2-yl]sulfonyl]phenyl]piperazin- | |
| | 1-yl]methanone | |
| 037 | 3-[4-(2-chloro-4-fluoro-benzoyl)piperazin-1-yl]-4- | * |
| | methoxy-N-methyl-N-(4,4,4- | |
| | trifluorobutyl)benzenesulfonamide | |
| 038 | (2-chloro-4-fluoro-phenyl)-[4-[2-methoxy-5-[[3- | *** |
| | benzyloxy-1-piperidyl]sulfonyl]phenyl]piperazin-1- | |
| | yl]methanone | |
| 039 | (2-chloro-4-fluoro-phenyl)-[4-[2-methoxy-5-[3- | *** |
| | benzyloxypyrrolidin-1-yl]sulfonyl-phenyl]piperazin-1- | |
| | yl]methanone | |
| 040 | [4-[5-[[2-benzyl-1,3,3a,4,6,6a-hexahydropyrrolo[3,4- | * |
| | c]pyrrol-5-yl]sulfonyl]-2-methoxy-phenyl]piperazin-1- | |
| | yl]-(2-chloro-4-fluoro-phenyl)methanone | |
| 041 | N,N-diethyl-3-[4-[4-fluoro-2- | * |
| | (trifluoromethyl)benzoyl]piperazin-1- | |
| | yl]benzenesulfonamide | |
| 042 | 4-fluoro-3-[4-[4-fluoro-2- | *** |
| | (trifluoromethyl)benzoyl]piperazin-1-yl]-N-methyl-N- | |
| | propyl-benzenesulfonamide | |
| 043 | 7-[4-[4-fluoro-2-(trifluoromethyl)benzoyl]piperazin-1- | *** |
| | yl]-N-methyl-N-propyl-2,3-dihydrobenzofuran-5- | |
| | sulfonamide | |
| 044 | 6-chloro-N,N-diethyl-5-[4-[4-fluoro-2- | * |
| | (trifluoromethyl)benzoyl]piperazin-1-yl]pyridine-3- | |
| | sulfonamide | |
| 045 | N,N-diethyl-5-[4-[4-fluoro-2- | * |
| | (trifluoromethyl)benzoyl]piperazin-1-yl]-6-methoxy- | |
| | pyridine-3-sulfonamide | |
| 046 | 3-[4-[4-fluoro-2-(trifluoromethyl)benzoyl]piperazin-1- | ** |
| | yl]-4-(2-methoxyethoxy)-N-methyl-N-propyl- | |
| | benzenesulfonamide | |
| 047 | 4-benzyloxy-3-[4-[4-fluoro-2- | *** |
| | (trifluoromethyl)benzoyl]piperazin-1-yl]-N-methyl-N- | |
| | propyl-benzenesulfonamide | |
| 048 | 3-[4-[4-fluoro-2-(trifluoromethyl)benzoyl]piperazin-1- | ** |
| | yl]-4-hydroxy-N-methyl-N-propyl-benzenesulfonamide | |
| 049 | 3-[4-[4-fluoro-2-(trifluoromethyl)benzoyl]piperazin-1- | * |
| | yl]-4-(2-hydroxyethoxy)-N-methyl-N-propyl- | |
| | benzenesulfonamide | |
| 050 | (2-chloro-4-fluoro-phenyl)-[4-[5-[[4-(4-fluorophenyl)-1- | **** |
| | piperidyl]sulfonyl]-2-methoxy-phenyl]piperazin-1- | |
| | yl]methanone | |
| 051 | (2-chloro-4-fluoro-phenyl)-[4-[5-[4-[2-(4- | *** |
| | chlorophenyl)ethyl]piperazin-1-yl]sulfonyl-2-methoxy- | |
| | phenyl]piperazin-1-yl]methanone | |
| 052 | (2-chloro-4-fluoro-phenyl)-[4-[5-[4-[2-(4- | *** |
| | fluorophenyl)ethyl]piperazin-1-yl]sulfonyl-2-methoxy- | |
| | phenyl]piperazin-1-yl]methanone | |
| 053 | (2-chloro-4-fluoro-phenyl)-[4-[2-methoxy-5-[4-[rac- | *** |
| | (2R)-2-phenylpropyl]piperazin-1-yl]sulfonyl- | |
| | phenyl]piperazin-1-yl]methanone | |
| 054 | (2-chloro-4-fluoro-phenyl)-[4-[2-methoxy-5-[4-[rac- | **** |
| | (2R)-2-(4-fluorophenyl)propyl]piperazin-1-yl]sulfonyl- | |
| | phenyl]piperazin-1-yl]methanone | |
| 055 | (2-chloro-4-fluoro-phenyl)-[4-[2-hydroxy-3-methyl-5- | **** |
| | [(4-phenyl-1-piperidyl)sulfonyl]phenyl]piperazin-1- | |
| | yl]methanone | |
| 056 | (2-chloro-4-fluoro-phenyl)-[4-[5-[[4-(4-fluorophenyl)-1- | **** |
| | piperidyl]sulfonyl]-2-hydroxy-3-methyl- | |
| | phenyl]piperazin-1-yl]methanone | |
| 057 | (2-chloro-4-fluoro-phenyl)-[4-[3-chloro-5-[(4-fluoro-1- | *** |
| | piperidyl)sulfonyl]-2-hydroxy-phenyl]piperazin-1- | |
| | yllmethanone | |
| 058 | N-tert-butyl-3-chloro-5-[4-(2-chloro-4-fluoro- | **** |
| | benzoyl)piperazin-1-yl]-4-hydroxy-benzenesulfonamide | |
| 059 | 3-chloro-5-[4-(2-chloro-4-fluoro-benzoyl)piperazin-1- | **** |
| | yl]-N-(cyclopropylmethyl)-4-hydroxy-N-methyl- | |
| | benzenesulfonamide | |
| 060 | (2-chloro-4-fluoro-phenyl)-[4-[5-(2,2- | *** |
| | dimethylpyrrolidin-1-yl)sulfonyl-2-methoxy- | |
| | phenyl]piperazin-1-yl]methanone | |

The above results clearly evidence that the tested compounds 001-060 have significant GFRα1-RET activity. Thus, the compounds of the invention are useful as neuroprotective and neurorestorative agents. The tested compounds 001-060 are well-representing the class of the compounds of formula (I).

### Example 3: Biological Activity of Comparative Compounds

The purpose of this experiment was to compare the GFRα1-RET activity of the compounds 001, 009 and 010 according to the invention to the GFRα1-RET activity of comparative compounds C01 and C02.

### Materials and Methods

Comparatives compounds C01 and C02 were prepared by using the same synthetic pathways and methods as described hereinabove for the compounds according to the invention, using the general knowledge in the art to adapt the reactants and experimental conditions as needed.

The structure of comparatives compounds C01 and C02 are presented on Table 5 below.

TABLE 5

| Cpd | Structure | Name |
|---|---|---|
| C01 | 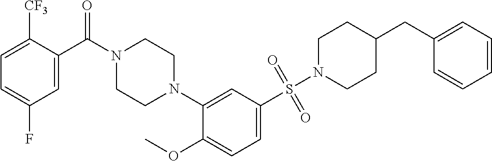 [SMILES: COc1ccc(S(=O)(=O)N2CCC(Cc3ccccc3)CC2)cc1N1CCN(C(=O)c2cc(F)ccc2C(F)(F)F)CC1] | (4-(5-((4- benzylpiperidin-1- yl)sulfonyl)-2- methoxyphenyl)piperazin- 1-yl)(5-fluoro-2- (trifluoromethyl)phenyl) methanone |
| C02 | 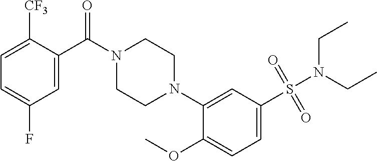 [SMILES: CCN(CC)S(=O)(=O)c1ccc(OC)c(N2CCN(C(=O)c3cc(F)ccc3C(F)(F)F)CC2)c1] | N,N-diethyl-3-(4-(5- fluoro-2- (trifluoromethyl)benzoyl) piperazin-1-yl)-4- methoxybenzenesulfonamide |

The compounds were tested for their activating activity of Elk1 signaling using the same materials and methods than for the compounds according to the invention (Example 2 herein, section “Materials and methods”).

### Results

The results for comparative compounds C01 and C02 are presented on Table 6 below. For easier comparison, the corresponding values from Table 4 above for compounds 009, 010 and 001 according to the invention were also reported in Table 6 (AL means “EC₅₀>50 μM”, * means “10 μM<EC₅₀<50 μM”, ** means “5 μM<EC₅₀<10 μM”, *** means “1 μM<EC₅₀<5 μM”, and **** means “EC₅₀<1 μM”).

TABLE 6

| | | Luciferase |
|---|---|---|
| Cpd | Name | Assay EC₅₀ |
| C01 | (4-(5-((4-benzylpiperidin-1-yl)sulfonyl)-2- | AL |
| | methoxyphenyl)piperazin-1-yl)(5-fluoro-2- | |
| | (trifluoromethyl)phenyl)methanone | |
| 009 | [4-[5-[(4-benzyl-1-piperidyl)sulfonyl]-2-methoxy- | **** |
| | phenyl]piperazin-1-yl]-(2-chloro-4,5-difluoro- | |
| | phenyl)methanone | |
| 010 | [4-[5-[(4-benzyl-1-piperidyl)sulfonyl]-2-methoxy- | *** |
| | phenyl]piperazin-1-yl]-[2-(difluoromethyl)-4-fluoro- | |
| | phenyl]methanone | |
| C02 | N,N-diethyl-3-(4-(5-fluoro-2- | AL |
| | (trifluoromethyl)benzoyl)piperazin-1-yl)-4- | |
| | methoxybenzenesulfonamide | |
| 001 | 3-[4-(2-chloro-4-fluoro-benzoyl)piperazin-1-yl]-N,N- | ** |
| | diethyl-4-methoxy-benzenesulfonamide | |

The above results clearly evidence that the compounds according to the invention have significantly higher GFRα1-RET activity than comparative compounds C01 and C02. Indeed, compounds C01 and C02 have an EC₅₀ higher than 50 μM, whereas compounds 001, 009 and 010 have an EC₅₀ lower than 10 μM, or even further lower. In the context of the medical uses according to the present invention, an ECs higher than 50 μM is actually quite high and might possibly be considered by a person skilled in the art “inactive” or “not active enough to be of therapeutical interest”.

It is noteworthy that the only structural difference between the compounds C01 and C02 and, respectively, compounds 009 and 010 or compound 001, is the substitution of the oxo-phenyl, i.e., the definitions of R^{A}-R^{D} substituents in formula (I). In particular, R^{D} does not represent CF₃ in compounds 009 and 010, but rather CHF₂ (010) or Cl (009), whereas in compound C01 the substituent that would correspond to R^{D} in formula (I) represents CF₃. Similarly, R^{D} does not represent CF₃ in compound 001, but rather Cl, whereas in compound C02 the substituent that would correspond to R^{D} in formula (I) represents CF₃.

Therefore, these results unambiguously evidence that the compounds of the invention are better activators of GFRα1-RET and thus better neuroprotective and neurorestorative candidates than the comparative compounds. Moreover, this surprising technical effect is directly associated with the specific structural feature of the compounds according to the invention.

1-15. (canceled)

16. A compound of formula (I-i): 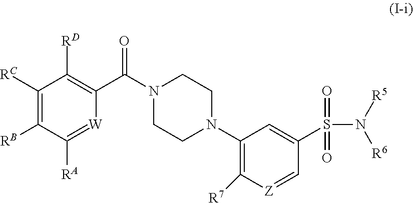
or a pharmaceutically acceptable salt and/or solvate thereof;
wherein:
W represents CH or N;
R^{A}, R^{B}, and R^{C} each independently represents hydrogen, F, Cl, CH₃, CF₃, CHF₂, or CH₂F;
R^{D} represents hydrogen, F, Cl, CH₃, CHF₂, or CH₂F;
R⁷ represents hydrogen, OH, halogen, (C₁-C₈) alkyl, cycloalkyl, (C₁-C₈) alkyl-O—, cycloalkyl-O—, cycloalkyl-(C₁-C₈) alkyl-O—, heterocycloalkyl-O—, heterocycloalkyl-(C₁-C₈) alkyl-O—, aryl-(C₁-C₈) alkyl-O—, heteroaryl-(C₁-C₈) alkyl-O—, R¹¹O—(C₁-C₈) alkyl-O—, R¹¹R¹²N—(C₁-C₈) alkyl-O—, (R¹¹O)(R¹²)N—(C₁-C₈) alkyl-O—, R¹¹R¹²N—(C₁-C₈) alkyl-, R¹¹O—(C₁-C₈) alkyl-, NR¹¹R¹², CN, CO₂H, CO₂R¹¹, CONH₂, CON(R¹¹)H, heterocycloalkyl, or heteroaryl; wherein R¹¹ and R¹² each independently represents hydrogen or (C₁-C₈) alkyl;
wherein the alkyl or cycloalkyl in R⁷ is optionally substituted by at least one F, Cl, OH, ═O, (C₁-C₈) alkyl, (C₁-C₈) alkyl-O—, heterocycloalkyl, aryl, or heteroaryl; and
wherein the heterocycloalkyl, aryl, or heteroaryl is optionally substituted by at least one F, Cl, (C₁-C₈) alkyl, CF₃, CHF₂, CH₂F, OCF₃, CN, OH, ═O, →O, (C₁-C₈) alkoxy, NR¹³R¹⁴, R¹³R¹⁴N—(C₁-C₈) alkyl-, R¹³O₂C(C₁-C₈) alkyl-, CO₂H, R¹³R¹¹N—C(O)—, R¹³O—NR¹⁴—, or (C₁-C₈) alkyl-CO₂—; wherein R¹³ and R¹⁴ each independently represents hydrogen or (C₁-C₈) alkyl;
Z represents C—H, C—R⁸, or N;
wherein R⁸ represents (C₁-C₄) alkyl, F, Cl, CF₃, CHF₂, CH₂F, OCF₃, CN, OH, or (C₁-C₄) alkoxy;
or Z represents C—R⁸ and R⁷ and R⁸ form together with the carbon atoms to which they are bound a cycloalkyl or heterocycloalkyl,
wherein the cycloalkyl or heterocycloalkyl is optionally substituted by at least one F, OH, ═O, →O, (C₁-C₈) alkyl, CF₃, HO₂C—CH₂—, (C₁-C₄) alkyl-CO₂—CH₂—, R¹⁵R¹⁶N—CH₂—, aryl, or aryl-(C₁-C₈) alkyl-, wherein R¹⁵ and R¹⁶ each independently represents hydrogen or (C₁-C₈) alkyl;
R⁵ represents hydrogen, (C₁-C₈) alkyl, CH₃ substituted by one to three (C₁-C₈) alkyl groups, cycloalkyl, heterocycloalkyl, aryl, or cycloalkyl-(C₁-C₈) alkyl;
wherein the alkyl or cycloalkyl in R⁵ is optionally substituted by at least one F, Cl, (C₁-C₈) alkyl, CF₃, OCF₃, CN, OH, ═O, (C₁-C₈) alkoxy, NR¹⁷R¹⁸, CO₂H, R¹⁷R¹⁸N—C(O)—, R¹⁷O—NR¹⁵—, heterocycloalkyl, aryl, or heteroaryl; wherein R¹⁷ and R¹⁸ each independently represents hydrogen or (C₁-C₈) alkyl; and
wherein the heterocycloalkyl, aryl, or heteroaryl is optionally substituted by at least one F, Cl, (C₁-C₈) alkyl, CF₃, CHF₂, CH₂F, OCF₃, CN, OH, ═O, →O, (C₁-C₈) alkoxy, NR¹⁹R²⁰, CO₂H, R¹⁹R²⁰N—C(O)—, R¹⁹O—NR²⁰O—, (C₁-C₈) alkyl-CO₂—, R¹⁹R²⁰N—(C₁-C₈) alkyl-, R¹⁹O₂C—(C₁-C₈) alkyl-, heterocycloalkyl, heteroaryl, aryl, or aryl-(C₁-C₈) alkyl-; wherein R¹⁹ and R²⁰ each independently represents hydrogen or (C₁-C₈) alkyl;
R⁶ represents (C₁-C₈) alkyl, CH₃ substituted by one to three (C₁-C₈) alkyl groups, cycloalkyl, heterocycloalkyl, aryl, or cycloalkyl-(C₁-C₈) alkyl;
wherein the alkyl or cycloalkyl in R⁶ is optionally substituted by at least one F, Cl, (C₁-C₈) alkyl, CF₃, OCF₃, CN, OH, ═O, (C₁-C₈) alkoxy, NR²¹R²², CO₂H, R²¹R²²N—C(O)—, R²¹O—NR²²—, heterocycloalkyl, aryl, or heteroaryl; wherein R²¹ and R²² each independently represents hydrogen or (C₁-C₈) alkyl; and
wherein the heterocycloalkyl, aryl or heteroaryl is optionally substituted by at least one F, Cl, (C₁-C₈) alkyl, CF₃, CHF₂, CH₂F, OCF₃, CN, OH, ═O, →O, (C₁-C₈) alkoxy, NR²³R²⁴, CO₂H, R²³R²⁴N—C(O)—, R²³O—NR²⁴—, (C₁-C₈) alkyl-CO₂—, R²³R²⁴N—(C₁-C₈) alkyl-, R²³OC—(C₁-C₈) alkyl-, heterocycloalkyl, heteroaryl, aryl, or aryl-(C₁-C₈) alkyl-; wherein R²³ and R²⁴ each independently represents hydrogen or (C₁-C₈) alkyl;
or R⁵ and R⁶ form together with the nitrogen atom to which they are bound an heterocycloalkyl,
wherein the heterocycloalkyl is optionally substituted by at least one F, Cl, (C₁-C₈) alkyl, CF₃, OCF₃, CN, OH, ═O, →O, (C₁-C₈) alkoxy, NR²⁵R²⁶, CO₂H, (C₁-C₈) alkyl-CO₂—, R²⁵R²⁶N—C(O)—, R²⁵O—NR²⁶—, R²⁵R²⁶N—(C₁-C₈) alkyl-, R²O₂C—(C₁-C₈) alkyl-, cycloalkyl, heterocycloalkyl, aryl, heteroaryl, cycloalkyl-(C₁-C₈) alkyl-, heterocycloalkyl-(C₁-C₈) alkyl-, aryl-(C₁-C₈) alkyl-, heteroaryl-(C₁-C₈) alkyl-, aryl-cycloalkyl-, cycloalkyl-O—, heterocycloalkyl-O—, aryl-O—, heteroaryl-O—, cycloalkyl-(C₁-C₈) alkyl-O—, heterocycloalkyl-(C₁-C₈) alkyl-O—, aryl-(C₁-C₈) alkyl-O—, heteroaryl-(C₁-C₈) alkyl-O—, cycloalkyl-NR²⁵—, heterocycloalkyl-NR²⁵—, aryl-NR²⁵—, heteroaryl-NR²⁵—, cycloalkyl-(C₁-C₈) alkyl-NR²⁵—, heterocycloalkyl-(C₁-C₈) alkyl-NR²⁵—, aryl-(C₁-C₈) alkyl-NR²⁵—, heteroaryl-(C₁-C₈) alkyl-NR²⁵—, benzylidene, heteroarylidene, aryl-(C₁-C₈) alkyl-ylidene-, or heteroaryl-(C₁-C₈) alkyl-ylidene-;
wherein R²⁵ and R²⁶ each independently represents hydrogen or (C₁-C₈) alkyl; and
wherein the heterocycloalkyl, aryl, heteroaryl, benzylidene, or heteroarylidene is optionally substituted at least one F, Cl, (C₁-C₈) alkyl, CF₃, CHF₂, CH₂F, OCF₃, CN, OH, ═O, →O, (C₁-C₈) alkoxy, NR²⁷R²⁸, CO₂H, R²⁷R²⁸SN—C(O)—, R²⁷O—NR²⁸—, (C₁-C₈) alkyl-CO₂—, R²⁷R²⁸SN—(C₁-C₈) alkyl-, R²⁷C—(C₁-C₈) alkyl-, heterocycloalkyl, heteroaryl, aryl, or aryl-(C₁-C₈) alkyl-; wherein R²⁷ and R²⁸ each independently represents hydrogen or (C₁-C₈) alkyl.

17. A compound of formula (I-ii) 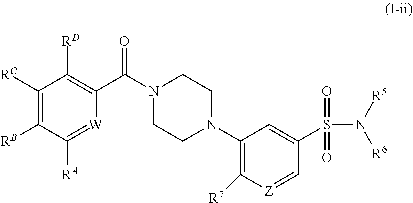
or a pharmaceutically acceptable salt and/or solvate thereof;
wherein:
W represents CH or N;
R^{A}, R^{B}, R^{C}, and R^{D} each independently represents hydrogen, F, Cl, CH₃, CF₃, CHF₂, or CH₂F;
R⁷ represents hydrogen, OH, halogen, (C₁-C₈) alkyl, cycloalkyl, (C₂-C₈) alkyl-O-cycloalkyl-O—, cycloalkyl-(C₁-C₈) alkyl-O—, heterocycloalkyl-O—, heterocycloalkyl-(C₁-C₈) alkyl-O—, aryl-(C₁-C₈) alkyl-O—, heteroaryl-(C₁-C₈) alkyl-O—, R¹¹O—(C₁-C₈) alkyl-O—, R¹¹R¹²N—(C₁-C₈) alkyl-O—, (R¹¹O)(R¹²)N—(C₁-C₈) alkyl-O—, R¹¹R¹²N—(C₁-C₈) alkyl-, R¹¹O—(C₁-C₈) alkyl-, NR¹¹R¹², CN, CO₂H, CO₂R¹¹, CONH₂, CON(R¹¹)H, heterocycloalkyl, or heteroaryl; wherein R¹¹ and R¹² each independently represents hydrogen or (C₁-C₈) alkyl;
wherein the alkyl or cycloalkyl in R⁷ is optionally substituted by at least one F, Cl, OH, ═O, (C₁-C₈) alkyl, (C₁-C₈) alkyl-O—, heterocycloalkyl, aryl, or heteroaryl; and
wherein the heterocycloalkyl, aryl, or heteroaryl is optionally substituted by at least one F, Cl, (C₁-C₈) alkyl, CF₃, CHF₂, CH₂F, OCF₃, CN, OH, ═O, →O, (C₁-C₈) alkoxy, NR¹³R¹⁴, R¹³R¹⁴N—(C₁-C₈) alkyl-, R¹³O₂C(C₁-C₈) alkyl-, CO₂H, R¹³R¹⁴N—C(O)—, R¹³O—NR¹⁴—, or (C₁-C₈) alkyl-CO₂—; wherein R¹³ and R⁴ each independently represents hydrogen or (C₁-C₈) alkyl;
Z represents C—H, C—R⁸, or N;
wherein R⁸ represents (C₁-C₄) alkyl, F, Cl, CF₃, CHF₂, CH₂F, OCF₃, CN, OH, or (C₁-C₄) alkoxy;
or Z represents C—R⁸ and R⁷ and R⁸ form together with the carbon atoms to which they are bound a cycloalkyl or heterocycloalkyl,
wherein the cycloalkyl or heterocycloalkyl is optionally substituted by at least one F, OH, ═O, →O, (C₁-C₈) alkyl, CF₃, HO₂C—CH₂—, (C₁-C₄) alkyl-CO₂—CH₂—, R¹⁵R¹⁶N—CH₂—, aryl, or aryl-(C₁-C₈) alkyl-, wherein R¹⁵ and R¹⁶ each independently represents hydrogen or (C₁-C₈) alkyl; and
R⁵ and R⁶ are each independently as defined above.

18. The compound according to claim 16, wherein W represents CH.

19. The compound according to claim 16, wherein:
at least one among R^{A}, R^{B}, R^{C} and R^{D} represents hydrogen; and/or
at least one among R^{A}, R^{B} and R^{D} represents F or Cl.

20. The compound according to claim 19, wherein at least one among R^{A} and R^{C} represents hydrogen.

21. The compound according to claim 19, wherein at least one among R^{B} and R^{D} represents F or Cl.

22. The compound according to claim 21, wherein R^{B} and R^{D} each independently represents F or Cl.

23. The compound according to claim 16, wherein R⁷ represents hydrogen, OH, halogen; (C₁-C₈) alkyl-O—, or cycloalkyl-O—, wherein the alkyl or cycloalkyl is optionally substituted by at least one F, Cl, OH, (C₁-C₈) alkoxy, or aryl.

24. The compound according to claim 23, wherein R⁷ represents OH, F; OCH₃, HO—CH₂—CH₂—O—, CH₃O—CH₂—CH₂—O—, or phenyl-CH₂—O—.

25. The compound according to claim 16, wherein Z represents C—R⁸, wherein R⁸ represents (C₁-C₄) alkyl, F, Cl, CF₃, CHF₂, CH₂F, OCF₃, CN, OH, or (C₁-C₄) alkoxy; or R⁷ and R⁸ form together with the carbon atoms to which they are bound a heterocycloalkyl; wherein the heterocycloalkyl is optionally substituted by at least one F, OH, ═O, (C₁-C₈) alkyl, CF₃, HO₂C—CH₂—, (C₁-C₄) alkyl-CO₂—CH₂—, or R¹⁵R¹⁶N—CH₂—, wherein R¹⁵ and R¹⁶ each independently represents hydrogen or (C₁-C₈) alkyl.

26. The compound according to claim 25, wherein R⁸ represents methyl or Cl.

27. The compound according to claim 16, wherein R⁵ represents hydrogen or (C₁-C₈) alkyl; and/or R⁶ represents (C₁-C₈) alkyl, cycloalkyl, heterocycloalkyl, cycloalkyl-(C₁-C₈) alkyl-, or aryl-(C₁-C₈) alkyl-, wherein the alkyl, cycloalkyl, heterocycloalkyl, or aryl is optionally substituted by at least one methyl, Cl, or F; or R⁵ and R⁶ form together with the nitrogen atom to which they are bound an heterocycloalkyl, wherein the heterocycloalkyl is optionally substituted by at least one F.

28. The compound according to claim 27, wherein R⁵ represents hydrogen, methyl or ethyl; and/or R⁶ represents ethyl, propyl, butyl, cyclopentyl, cyclohexyl, 2-adamantyl, 3-methyloxetan-3-yl, cyclopropyl-CH₂—, cyclobutyl-CH₂—, cyclohexyl-CH₂—, phenyl-CH₂— (benzyl), 3-chlorophenyl-CH₂—, or 4-fluorophenyl-CH₂—; or R⁵ and R⁶ form together with the nitrogen atom to which they are bound a pyrrolidine, 4-benzyl-piperidine, 4-phenyl-4-hydroxy-piperidine, 4-benzyl-4-hydroxy-piperidine, 4-benzyl-piperazine, tert-butyl 1,3,3a,4,6,6a-hexahydropyrrolo[3,4-c]pyrrole-2-carboxylate, 4-benzylidene-1-piperidine, 4-(2-phenylethyl)-piperidine, 4-phenylpiperidine, 3-phenylpiperidine, 3-benzyl-piperidine, 3-phenylpyrrolidin, 3-benzylpyrrolidine, 4-trifluoromethylpiperidine, 2-azabicyclo[2.2.1]heptane, 3-benzyloxypiperidine, 3-benzyloxypyrrolidine, 2-benzyl-1,3,3a,4,6,6a-hexahydropyrrolo[3,4-c]pyrrolidine, 4-(4-fluorophenyl)piperidine, [2-(4-chlorophenyl)ethyl]piperazine, [2-(4-fluorophenyl)ethyl]piperazine, 2-(phenylpropyl)piperazine, [2-(4-fluorophenyl)propyl]piperazine, 4-fluoropiperidine, or 2,2-dimethylpyrrolidine.

29. The compound according to claim 16, wherein said compound is selected from the group consisting of:

| | |
|---|---|
| 001 | 3-[4-(2-chloro-4-fluoro-benzoyl)piperazin-1-yl]-N,N- |
| | diethyl-4-methoxy-benzenesulfonamide |
| 002 | 3-[4-(2-chloro-4,5-difluoro-benzoyl)piperazin-1-yl]-N,N- |
| | diethyl-4-methoxy-benzenesulfonamide |
| 003 | (2,4-dichlorophenyl)-[4-(2-methoxy-5-pyrrolidin-1- |
| | ylsulfonyl-phenyl)piperazin-1-yl]methanone |
| 004 | (2-chloro-4-fluoro-phenyl)-[4-(2-methoxy-5-pyrrolidin-1- |
| | ylsulfonyl-phenyl)piperazin-1-yl]methanone |
| 005 | 3-[4-(2-chloro-4,5-difluoro-benzoyl)piperazin-1-yl]-4- |
| | methoxy-N-methyl-N-propyl-benzenesulfonamide |
| 006 | 3-[4-[2-(difluoromethyl)-4-fluoro-benzoyl]piperazin-1- |
| | yl]-4-methoxy-N-methyl-N-propyl-benzenesulfonamide |
| 007 | [4-[5-[(4-benzyl-1-piperidyl)sulfonyl]-2-methoxy- |
| | phenyl]piperazin-1-yl]-[2-(difluoromethyl)phenyl]methanone |
| 008 | [4-[5-[(4-benzyl-1-piperidyl)sulfonyl]-2-methoxy- |
| | phenyl]piperazin-1-yl]-(3-chloro-5-fluoro-2-pyridyl)methanone |
| 009 | [4-[5-[(4-benzyl-1-piperidyl)sulfonyl]-2-methoxy- |
| | phenyl]piperazin-1-yl]-(2-chloro-4,5-difluoro- |
| | phenyl)methanone |
| 010 | [4-[5-[(4-benzyl-1-piperidyl)sulfonyl]-2-methoxy- |
| | phenyl]piperazin-1-yl]-[2-(difluoromethyl)-4-fluoro- |
| | phenyl]methanone |
| 011 | (2-chloro-4-fluoro-phenyl)-[4-[5-[(4-hydroxy-4-phenyl-1- |
| | piperidyl)sulfonyl]-2-methoxy-phenyl]piperazin-1- |
| | yl]methanone |
| 012 | [4-[5-[(4-benzyl-4-hydroxy-1-piperidyl)sulfonyl]-2- |
| | methoxy-phenyl]piperazin-1-yl]-(2-chloro-4-fluoro- |
| | phenyl)methanone |
| 013 | [4-[5-(4-benzylpiperazin-1-yl)sulfonyl-2-methoxy- |
| | phenyl]piperazin-1-yl]-(2-chloro-4-fluoro-phenyl)methanone |
| 014 | 3-[4-(2-chloro-4-fluoro-benzoyl)piperazin-1-yl]-N- |
| | (cyclobutylmethyl)-4-methoxy-N-methyl-benzenesulfonamide |
| 015 | 3-[4-(2-chloro-4-fluoro-benzoyl)piperazin-1-yl]-N- |
| | (cyclohexylmethyl)-4-methoxy-N-methyl-benzenesulfonamide |
| 016 | 3-[4-(2-chloro-4-fluoro-benzoyl)piperazin-1-yl]-N- |
| | cyclopentyl-4-methoxy-N-methyl-benzenesulfonamide |
| 017 | 3-[4-(2-chloro-4-fluoro-benzoyl)piperazin-1-yl]-N- |
| | cyclohexyl-4-methoxy-N-methyl-benzenesulfonamide |
| 018 | 3-[4-(2-chloro-4-fluoro-benzoyl)piperazin-1-yl]-4- |
| | methoxy-N-methyl-N-(3-methyloxetan-3- |
| | yl)benzenesulfonamide |
| 019 | N-benzyl-3-[4-(2-chloro-4-fluoro-benzoyl)piperazin-1- |
| | yl]-4-methoxy-N-methyl-benzenesulfonamide |
| 020 | 3-[4-(2-chloro-4-fluoro-benzoyl)piperazin-1-yl]-N-[(3- |
| | chlorophenyl)methyl]-4-methoxy-N-methyl- |
| | benzenesulfonamide |
| 021 | 3-[4-(2-chloro-4-fluoro-benzoyl)piperazin-1-yl]-N-[(4- |
| | fluorophenyl)methyl]-4-methoxy-N-methyl- |
| | benzenesulfonamide |
| 022 | tert-butyl 5-[3-[4-(2-chloro-4-fluoro-benzoyl)piperazin-1- |
| | yl]-4-methoxy-phenyl]sulfonyl-1,3,3a,4,6,6a- |
| | hexahydropyrrolo[3,4-c]pyrrole-2-carboxylate |
| 023 | [4-[5-[(4-benzylidene-1-piperidyl)sulfonyl]-2-methoxy- |
| | phenyl]piperazin-1-yl]-(2-chloro-4-fluoro-phenyl)methanone |
| 024 | (2-chloro-4-fluoro-phenyl)-[4-[2-methoxy-5-[[4-(2- |
| | phenylethyl)-1-piperidyl]sulfonyl]phenyl]piperazin-1- |
| | yl]methanone |
| 025 | (2-chloro-4-fluoro-phenyl)-[4-[2-methoxy-5-[(4-phenyl- |
| | 1-piperidyl)sulfonyl]phenyl]piperazin-1-yl]methanone |
| 026 | (2-chloro-4-fluoro-phenyl)-[4-[2-methoxy-5-[[3-phenyl- |
| | 1-piperidyl]sulfonyl]phenyl]piperazin-1-yl]methanone |
| 027 | (2-chloro-4-fluoro-phenyl)-[4-[2-methoxy-5-[[3-benzyl- |
| | 1-piperidyl]sulfonyl]phenyl]piperazin-1-yl]methanone |
| 028 | (2-chloro-4-fluoro-phenyl)-[4-[2-methoxy-5-[3- |
| | phenylpyrrolidin-1-yl]sulfonyl-phenyl]piperazin-1- |
| | yl]methanone |
| 029 | (2-chloro-4-fluoro-phenyl)-[4-[2-methoxy-5-[3- |
| | benzylpyrrolidin-1-yl]sulfonyl-phenyl]piperazin-1- |
| | yl]methanone |
| 030 | 3-[4-(2-chloro-4-fluoro-benzoyl)piperazin-1-yl]-4- |
| | methoxy-N-methyl-N-propyl-benzenesulfonamide |
| 031 | (2-chloro-4-fluoro-phenyl)-[4-[2-methoxy-5-[[4- |
| | (trifluoromethyl)-1-piperidyl]sulfonyl]phenyl]piperazin-1- |
| | yl]methanone |
| 032 | N-tert-butyl-3-[4-(2-chloro-4-fluoro-benzoyl)piperazin-1- |
| | yl]-4-methoxy-benzenesulfonamide |
| 033 | N-tert-butyl-3-[4-(2-chloro-4-fluoro-benzoyl)piperazin-1- |
| | yl]-4-methoxy-N-methyl-benzenesulfonamide |
| 034 | N-(2-adamantyl)-3-[4-(2-chloro-4-fluoro- |
| | benzoyl)piperazin-1-yl]-4-methoxy-benzenesulfonamide |
| 035 | 3-[4-(2-chloro-4-fluoro-benzoyl)piperazin-1-yl]-4- |
| | methoxy-N-(1-methylcyclohexyl)benzenesulfonamide |
| 036 | (2-chloro-4-fluoro-phenyl)-[4-[2-methoxy-5-[[2- |
| | azabicyclo[2.2.1]heptan-2-yl]sulfonyl]phenyl]piperazin-1- |
| | yl]methanone |
| 037 | 3-[4-(2-chloro-4-fluoro-benzoyl)piperazin-1-yl]-4- |
| | methoxy-N-methyl-N-(4,4,4- |
| | trifluorobutyl)benzenesulfonamide |
| 038 | (2-chloro-4-fluoro-phenyl)-[4-[2-methoxy-5-[[3- |
| | benzyloxy-1-piperidyl]sulfonyl]phenyl]piperazin-1- |
| | yl]methanone |
| 039 | (2-chloro-4-fluoro-phenyl)-[4-[2-methoxy-5-[3- |
| | benzyloxypyrrolidin-1-yl]sulfonyl-phenyl]piperazin-1- |
| | yl]methanone |
| 040 | [4-[5-[2-benzyl-1,3,3a,4,6,6a-hexahydropyrrolo[3,4- |
| | c]pyrrol-5-yl]sulfonyl]-2-methoxy-phenyl]piperazin-1-yl]-(2- |
| | chloro-4-fluoro-phenyl)methanone |
| 041 | N,N-diethyl-3-[4-[4-fluoro-2- |
| | (trifluoromethyl)benzoyl]piperazin-1-yl]benzenesulfonamide |
| 042 | 4-fluoro-3-[4-[4-fluoro-2- |
| | (trifluoromethyl)benzoyl]piperazin-1-yl]-N-methyl-N-propyl- |
| | benzenesulfonamide |
| 043 | 7-[4-[4-fluoro-2-(trifluoromethyl)benzoyl]piperazin-1- |
| | yl]-N-methyl-N-propyl-2,3-dihydrobenzofuran-5-sulfonamide |
| 044 | 6-chloro-N,N-diethyl-5-[4-[4-fluoro-2- |
| | (trifluoromethyl)benzoyl]piperazin-1-yl]pyridine-3- |
| | sulfonamide |
| 046 | 3-[4-[4-fluoro-2-(trifluoromethyl)benzoyl]piperazin-1- |
| | yl]-4-(2-methoxyethoxy)-N-methyl-N-propyl- |
| | benzenesulfonamide |
| 047 | 4-benzyloxy-3-[4-[4-fluoro-2- |
| | (trifluoromethyl)benzoyl]piperazin-1-yl]-N-methyl-N-propyl- |
| | benzenesulfonamide |
| 048 | 3-[4-[4-fluoro-2-(trifluoromethyl)benzoyl]piperazin-1- |
| | yl]-4-hydroxy-N-methyl-N-propyl-benzenesulfonamide |
| 049 | 3-[4-[4-fluoro-2-(trifluoromethyl)benzoyl]piperazin-1- |
| | yl]-4-(2-hydroxyethoxy)-N-methyl-N-propyl- |
| | benzenesulfonamide |
| 050 | (2-chloro-4-fluoro-phenyl)-[4-[5-[4-(4-fluorophenyl)-1- |
| | piperidyl]sulfonyl]-2-methoxy-phenyl]piperazin-1- |
| | yl]methanone |
| 051 | (2-chloro-4-fluoro-phenyl)-[4-[5-[4-[2-(4- |
| | chlorophenyl)ethyl]piperazin-1-yl]sulfonyl-2-methoxy- |
| | phenyl]piperazin-1-yl]methanone |
| 052 | (2-chloro-4-fluoro-phenyl)-[4-[5-[4-[2-(4- |
| | fluorophenyl)ethyl]piperazin-1-yl]sulfonyl-2-methoxy- |
| | phenyl]piperazin-1-yl]methanone |
| 053 | (2-chloro-4-fluoro-phenyl)-[4-[2-methoxy-5-[4-[rac-(2R)- |
| | 2-phenylpropyl]piperazin-1-yl]sulfonyl-phenyl]piperazin-1- |
| | yl]methanone |
| 054 | (2-chloro-4-fluoro-phenyl)-[4-[2-methoxy-5-[4-[rac-(2R)- |
| | 2-(4-fluorophenyl)propyl]piperazin-1-yl]sulfonyl- |
| | phenyl]piperazin-1-yl]methanone |
| 055 | (2-chloro-4-fluoro-phenyl)-[4-[2-hydroxy-3-methyl-5- |
| | [(4-phenyl-1-piperidyl)sulfonyl]phenyl]piperazin-1- |
| | yl]methanone |
| 056 | (2-chloro-4-fluoro-phenyl)-[4-[5-[[4-(4-fluorophenyl)-1- |
| | piperidyl]sulfonyl]-2-hydroxy-3-methyl-phenyl]piperazin-1- |
| | yl]methanone |
| 057 | (2-chloro-4-fluoro-phenyl)-[4-[3-chloro-5-[(4-fluoro-1- |
| | piperidyl)sulfonyl]-2-hydroxy-phenyl]piperazin-1- |
| | yl]methanone |
| 058 | N-tert-butyl-3-chloro-5-[4-(2-chloro-4-fluoro- |
| | benzoyl)piperazin-1-yl]-4-hydroxy-benzenesulfonamide |
| 059 | 3-chloro-5-[4-(2-chloro-4-fluoro-benzoyl)piperazin-1-yl]- |
| | N-(cyclopropylmethyl)-4-hydroxy-N-methyl- |
| | benzenesulfonamide |
| 060 | (2-chloro-4-fluoro-phenyl)-[4-[5-(2,2-dimethylpyrrolidin- |
| | 1-yl)sulfonyl-2-methoxy-phenyl]piperazin-1-yl]methanone |

and pharmaceutically acceptable salts and/or solvates thereof.

30. A process for manufacturing a compound according to claim 16, wherein said process comprises a step of reaction of:
a compound of formula (II) 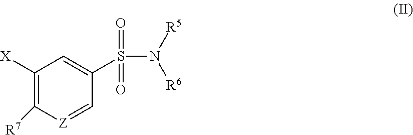
wherein:
Z, R⁵, R⁶ and R⁷ are either as defined above, and
X represents halide or —CF₃SO₃,
with a compound of formula (III) 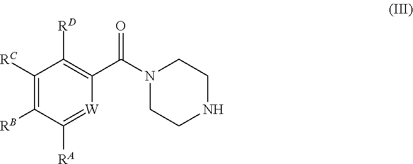
wherein W and R^{A}-R^{D} are either as defined above
in presence of a base and a metal catalyst;
thereby obtaining either the compound of formula (I-i) or the pharmaceutically acceptable salt and/or solvate thereof.

31. A process for manufacturing a compound according to claim 17, wherein said process comprises a step of reaction of:
a compound of formula (II) 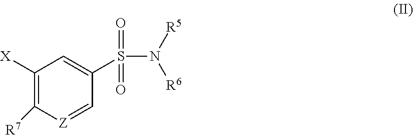
wherein
Z, R⁵, R⁶ and R⁷ are either as defined above, and
X represents halide or —CF₃SO₃,
with a compound of formula (III) 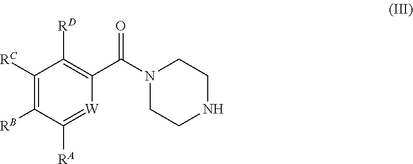
wherein W and R^{A}-R^{D} are either as defined above,
in presence of a base and a metal catalyst;
thereby obtaining either the compound of formula (I-ii) or the pharmaceutically acceptable salt and/or solvate thereof.
